(19) United States
(12) Patent Application Publication
(10) Pub. No.: US 2014/0322227 A1
(43) Pub. Date: Oct. 30, 2014

(54) PEPTIDES FOR TREATMENT OF BONE DEFICIENCY AND AUTOIMMUNE DISORDERS

(71) Applicants: H. LEE MOFFITT CANCER CENTER & RESEARCH INSTITUTE, INC., TAMPA, FL (US); UNIVERSITY OF SOUTH FLORIDA, TAMPA, FL (US); MODULATION THERAPEUTICS, TAMPA, FL (US)

(72) Inventors: LORI HAZLEHURST, RUSKIN, FL (US); MARK MCLAUGHLIN, TAMPA, FL (US); RAJESH R. NAIR, TAMPA, FL (US)

(21) Appl. No.: 14/263,631

(22) Filed: Apr. 28, 2014

Related U.S. Application Data
(60) Provisional application No. 61/816,580, filed on Apr. 26, 2013.

(51) Int. Cl. C07K 7/64; A61K 38/12; A61K 45/06

(52) U.S. Cl. CPC C07K 7/64; A61K 45/06; A61K 38/12
USPC 424/142.1; 514/13.5; 514/16.7; 514/21.2; 514/3.8; 514/1.9; 514/6.9; 514/4.2; 514/21.1

(57) ABSTRACT

The present invention concerns the use of HYD1 peptides to reduce activated T-cell numbers and/or to promote bone preservation in vivo. The present invention concerns methods of treating a bone deficiency and/or an autoimmune disorder, comprising administering an effective amount of a HYD1 peptide. Another aspect of the invention concerns a pharmaceutical composition comprising a HYD1 peptide and another agent for treating a bone deficiency and/or another agent for treating an autoimmune disorder.

### CROSS-REFERENCE TO RELATED APPLICATION

The present application claims the benefit of U.S. Provisional Application No. 61/816,580, filed Apr. 26, 2013, which is hereby incorporated by reference herein in its entirety, including any figures, tables, nucleic acid sequences, amino acid sequences, or drawings.

### GOVERNMENT SUPPORT

This invention was made with government support under grant number CA171367 awarded by the National Institutes of Health. The government has certain rights in the invention.

### BACKGROUND OF THE INVENTION

Lymphocytes originate from stem cells in bone marrow. The lymphocytes appear to be attracted to the thymus by chemotactic factors. Those lymphocytes that enter the thymus mature and develop into activated T-lymphocytes or activated T-cells. The activated T-cells are able to respond to antigens encountered elsewhere in the body. The T-cells can divide into two groups: (1) a group that enters the blood, some of which remain in circulation and some lodge in other lymphoid tissue; and (2) a group that remains in the thymus gland and are the source of future generations of T-cells.

T-cells are believed to carry out three defensive functions: (1) they stimulate the production and growth of antibodies by other lymphocytes; (2) they stimulate the growth and action of phagocytes, which surround and engulf invading viruses and microbes; and (3) they recognize and destroy foreign and abnormal tissue. Thus, the thymus gland plays a critically important role in the body's response to disease invasion. The thymus gland and T-cell function play such a pivotal and important role in generating and regulating immune response that a deficiency or imbalance in their function will cause immune system dysfunction.

Autoimmune disorders are caused by an immune response against the body's own normal cells or tissues. Autoimmune disorders result in destruction of one or more types of body tissues, abnormal growth of an organ or organs, or changes in organ function or functions. The disorders may affect only one organ or tissue type or may affect multiple organs and tissue types. In addition, a person may experience one or more autoimmune disorders at the same time. Organs and tissues commonly affected by autoimmune disorders include blood components such as red blood cells, blood vessels, connective tissues, endocrine glands such as the thyroid or pancreas, muscles, joints, skin, and bone.

Bone is generally believed to be a tissue in a steady state, in which bone formation by osteoblasts and bone resorption by osteoclasts occur continuously. Osteoporosis is a disease that frequently occurs in menopausal and post-menopausal women, and is characterized by an imbalance of activity of osteoclasts and osteoblasts. Bone tissue provides support for the body and includes mineral (including calcium and phosphorous), a matrix of collagenous and noncollagenous proteins, and cells. Living bone tissue exhibits a dynamic equilibrium between formation of bone, which is called deposition, and break-down of bone, which is called resorption. Three types of cells found in bone, osteocytes, osteoblasts and osteoclasts, are involved in this equilibrium. Osteoblasts promote formation of bone tissue, whereas osteoclasts are associated with resorption. Resorption, which is the dissolution of bone matrix and mineral, is a fast and efficient process relative to bone formation and can release large amounts of mineral from bone. Osteoclasts play a role in the regulation of the normal remodeling of skeletal tissue and in resorption induced by hormones.

After skeletal maturity, the amount of bone in the skeleton reflects the balance (or imbalance) of bone formation and bone resorption. Peak bone mass occurs after skeletal maturity prior to the fourth decade of life. Between the fourth and fifth decades, the equilibrium shifts and bone resorption dominates. The inevitable decrease in bone mass with advancing years starts earlier in females than males and is distinctly accelerated after menopause in some females.

Osteopenia is a condition relating generally to any decrease in bone mass to below normal levels. Such a condition may arise from a decrease in the rate of bone synthesis or an increase in the rate of bone destruction or both. A common form of osteopenia is primary osteoporosis, also referred to as postmenopausal and senile osteoporosis. This form of osteoporosis is a consequence of the universal loss of bone with age and is often a result of increase in bone resorption with a normal rate of bone formation.

### BRIEF SUMMARY OF THE INVENTION

The present invention concerns the use of HYD1 peptides to reduce activated T-cell numbers and promote bone preservation in vivo. In particular, an aspect of the invention concerns a method for reducing the number of activated T-cells (e.g., CD4⁺, CD8⁺, regulatory T cells (T_{reg})) in a subject, comprising administering an effective amount of a HYD1 peptide to the subject. In some embodiments, the subject has, or is at risk of developing, an autoimmune disorder. Another aspect of the invention concerns a method for promoting bone preservation in a subject, comprising administering an effective amount of a HYD1 peptide to the subject. In some embodiments, the subject has, or is at risk of developing, a bone deficiency.

Another aspect of the invention includes a method for treating bone deficiency and/or an autoimmune disorder in a subject, comprising administering an effective amount of a HYD1 peptide to the subject.

Another aspect of the invention is a pharmaceutical composition comprising a HYD1 peptide and one or more agents selected from an agent for treatment of a bone deficiency and/or an autoimmune disorder. The pharmaceutical composition may be administered to a subject in accordance with methods of the invention.

### BRIEF DESCRIPTION OF THE DRAWINGS

FIGS. 1A-1C show the effect of a cyclic HYD1 peptide (MTI-101) on the CD4 T-cell population and its active subset in the bone marrow (FIG. 1C), spleen (FIG. 1B), and lymph node (FIG. 1A) compartments of mice. All MTI-101-treated mice (MTI-101) and PBS-treated mice (V.C.) were treated via intraperitoneal injections 3 times a week (Monday, Wednesday and Friday) starting 6 days post-5TGM1 cell injection and was continued till the end of the experiment dictated by appearance of hind leg paralysis in the PBS treated mice. The sham mice cohorts were left untreated. Processing of cells was performed as described in Example 1. Cell surface staining for CD3 and CD4 double positivity in cells represent the total CD4 population whereas a subset within this population that were also CD69 positive (CD3, CD4 and CD69 triple positivity) represents the activated CD4 cell subset population. Acquired data from LSRII was analyzed using FLOWJO software. All analysis was performed on live cells by gating out the dead cell population. The experiments were performed three independent times and the data is represented as mean±SEM.

FIGS. 2A-2C show the effect of a cyclic HYD1 peptide (MTI-101) on the CD8 T-cell population and its active subset in the bone marrow (FIG. 2C), spleen (FIG. 2A), and lymph node (FIG. 2B) compartments of mice. All MTI-101-treated mice (MTI-101) and PBS treated mice (V.C.) were treated via intraperitoneal injections 3 times a week (Monday, Wednesday and Friday) starting 6 days post-5TGM1 cell injection and was continued till the end of the experiment dictated by appearance of hind leg paralysis in the PBS treated mice. The sham mice cohorts were left untreated. Processing of cells was performed as described in Example 1. Cell surface staining for CD3 and CD8 double positivity in cells represents the total CD8 population whereas a subset within this population that were also CD69 positive (CD3, CD8 and CD69 triple positivity) represents the activated CD8 cell subset population. Acquired data from LSRII was analyzed using FLOWJO software. All analysis was performed on live cells by gating out the dead cell population. The experiments were performed three independent times and the data is represented as mean±SEM.

FIG. 3 shows the effect of a cyclic HYD1 peptide (MTI-101) on the regulatory T cells (Treg) in the bone marrow, spleen, and lymph node compartments of mice. All MTI-101-treated mice (MTI-101) and PBS-treated mice (V.C.) were treated via intraperitoneal injections 3 times a week (Monday, Wednesday and Friday) starting 6 days post-5TGM1 cell injection and was continued till the end of the experiment dictated by appearance of hind leg paralysis in the PBS treated mice. The sham mice cohorts were left untreated. Processing of cells was performed as described in Example 3. Cell surface staining for CD3 and CD4 double positivity in cells represent the total CD4 population whereas a subset within this population that were also double positive for cell surface marker CD25 and intracellular marker Foxp3 represents the active Treg cell population. Acquired data from LSRII was analyzed using Flowjo software. All analysis was performed on live cells by gating out the dead cell population. The experiments were performed three independent times and the data is represented as mean±SEM.

FIG. 4 the effect of a cyclic HYD1 peptide (MTI-101) on alkaline phosphatase (ALP) in mouse pre-osteoblastic MC3T3-E1 cells. Cells were cultured in regular media (RM), 0.195 to 50 μM of MTI-101 or differentiation media (DM) as positive control. ALP activity was expressed as mean fold-change ±S.D. Significant difference was determined using ANOVA with post hoc Dunnet's test (n=3; * p<0.05 and # p>0.05).

FIGS. 5A-5D show the effect of a cyclic HYD1 peptide (MTI-101) on RAW 264.7 cells (FIGS. 5A and 5B) and on RAW 264.7 cell-differentiated osteoclasts (FIGS. 5C and 5D). RAW 264.7 cells were treated with 6.25 to 50 μM MTI-101 and at the end of 24 hrs the cells were scrapped and spun down. After a single wash the cells were stained with Annexin V-FITC dye. The resulting Annexin V positivity was determined using Calibur Flow cytometry. RAW 264.7 cells were treated with 6.25 to 50 μM and incubated for 72 hrs. At the end of the incubation 2 mg/ml of 3-(4,5-dimethythiazol-2-yl)-2,5-diphenyl tetrazolium bromide (MTT). After 4 hours incubation, the resulting formazan product was solubilized by addition of DMSO and the resulting color was measured by a plate reader. RAW 264.7 cells were stimulated with 50 ng/ml RANKL to differentiate into osteoclasts, and different doses of MTI-101 were added into the medium on day 6. After 24 hours of treatment, the cells were stained for TRAP activity. For quantification, the cells in each treated well were manually counted using a light microscope (FIG. 5C). FIG. 5D are micrographs of osteoclasts treated with the indicated doses of MTI-101. All the results in FIGS. 5A-5C are plotted as mean±S.D and are a representative of three independently performed experiments.

FIGS. 6A-6C show the effect of cyclic peptide (MTI-101) on RAW 264.7 cells and on RAW 264.7 cell-differentiated osteoclasts. RAW 264.7 cells were treated with 6.25 to 50 μM MTI-101 on days 1, 3 and 5, and on day 7 the cells were scrapped and spun down. After a single wash the cells were stained with Annexin V-FITC dye. The resulting Annexin V positivity was determined using Calibur Flow cytometry. (B) RAW 264.7 cells were treated with 6.25 to 50 μM on day 1, 3 and 5. On day 7, 2 mg/ml of 3-(4,5-dimethythiazol-2-yl)-2,5-diphenyl tetrazolium bromide (MTT). After 4 hours incubation the resulting formazan product was solubilized by addition of DMSO and the resulting color was measured by a plate reader. (C) RAW 264.7 cells were stimulated with 50 ng/ml RANKL in the presence or absence of different doses of MTI-101 on day 1, 3 and 5. After 7 days, the cells were stained for TRAP activity. For quantification, the cells in each treated well were manually counted using a light microscope. All the results are plotted as mean±S.D and are a representative of three independently performed experiments.

FIG. 7 shows the chemical structure of MTI-101, which is also shown in an alternative representation in FIG. 23. It will be understood that, in the representation in FIG. 7, each amino acid abbreviation (e.g., Lys, Leu, Trp, Ala, etc.) is referring to the substituent of the indicated amino acid. The remaining portion of each amino acid is within the adjacent portion of the ring scaffold.

FIGS. 8-33 show chemical structures of some embodiments of cyclic HYD1 peptides useful in the invention.

FIG. 34 shows a formula for some embodiments of cyclic HYD1 peptides useful in the invention, wherein R₁ is K; R₂ is L; R₃ is K; R₄ is L; R₅ is K; R₆ is selected from the group consisting of W, A, and M; R₇ is selected from the group consisting of S, A, Y, and V; R₈ is selected from the group consisting of V and A; R₉ is selected from the group consisting of V, A, and S; and R₁₀ is selected from the group consisting of M, A, W, and nor-Leu. It will be understood that, in the representation in FIG. 34, each R group is the substituent of the indicated amino acid. The remaining portion of each amino acid is within the adjacent portion of the ring scaffold. For example, R₅ is the substituent of K, and R₆ is selected from the group of substituents consisting of W, A, and M.

FIGS. 35-37 show linkers (beta turn promoters) useful in linking amino acid sequences to produce cyclic HYD1 peptides useful in the invention.

FIG. 38 shows the chemical structure of a cyclic HYD1 peptide useful in the invention. It will be understood that, in the representation in FIG. 38, each amino acid abbreviation (e.g., Lys, Leu, Trp, Ser, etc.) is referring to the substituent of the indicated amino acid. The remaining portion of each amino acid is within the adjacent portion of the ring scaffold.

FIGS. 39-41 show formulas for some embodiments of cyclic HYD1 peptides useful in the invention, wherein R¹ through R⁵ and R⁶ through R¹⁰ are substituents of natural or unnatural amino acids, wherein a sequence of amino acids with R¹ through R⁵ is a non-recognition sequence and a sequence of amino acids with R⁶ through R¹⁰ is a recognition sequence.

FIG. 42. CD11b cells were isolated using immunomagnetic beads from the tibia and femur of B6.Cg-Cd44tm1Hbg/J and wild type C57BL/6J mice. Isolated cells were initially expanded in M-CSF for 7 days, then RANKL was added to induce differentiation to osteoclasts lineage. On the 5^{th} day when mature osteoclasts were observed, cultures were treated with varying concentrations of MTI-101 for 24 hours. Osteoclasts were visualized using TRAPc positivity and counted (n=3 independent experiments performed in triplicates).

FIG. 43. MTI-101 treated mice show increased trabecular bone density compared to tumor-bearing vehicle control-treated mice (VC). The same 10 tibia bones imaged by Faxitron in FIG. 43 were imaged with a Micro PET/SPECT/CT (Siemens Inveon). After imaging, the bones were reconstructed using COBRA (Constraint Based Reconstruction and Analysis) and analyzed with the Siemens Inveon workstation. 30 separate longitudinal images for each tibia were measured for both bone volume and total volume with the indicated parameters (2 mm below growth plate to 2 mm above junction with fibula). All 30 images were combined and the tibia were analyzed by bone volume/total volume (BV/TV). Images of the control group were compared to both MTI-101 and bortezomib treated mice. MTI-101 treated mice had a p-value of 0.048 while bortezomib treated mice had a p-value of 0.055 (students t-test). The same bones that were assayed for tumor volume in FIG. 43 were used to determine bone density by CT scanning.

### DETAILED DESCRIPTION OF THE INVENTION

The present invention concerns the use of HYD1 peptides to reduce activated T-cell numbers and promote bone preservation in vivo. In particular, an aspect of the invention concerns a method for reducing the number of activated T-cells in a subject, comprising administering an effective amount of a HYD1 peptide to the subject. For example, the method may reduce the number of activated T-cell populations in a subject as described in the Examples herein. In some embodiments, the subject has, or is at risk of developing, an autoimmune disorder. Another aspect of the invention concerns a method for promoting bone preservation in a subject, comprising administering an effective amount of a HYD1 peptide to the subject. In some embodiments, the subject has, or is at risk of developing, a bone deficiency.

Another aspect of the invention includes a method for treating bone deficiency and/or an autoimmune disorder in a subject, comprising administering an effective amount of a HYD1 peptide to the subject.

In some embodiments of the aforementioned methods of the invention, the subject does not have a proliferation disorder, such as cancer. In some embodiments of the aforementioned of the invention, the subject does have a proliferation disorder, such as cancer.

Another aspect of the invention is a pharmaceutical composition comprising a HYD1 peptide and one or more agents selected from an agent for treatment of a bone deficiency and/or an autoimmune disorder. Preferably, the composition includes a pharmaceutically acceptable carrier or diluent. The pharmaceutical composition may be administered to a subject in accordance with methods of the invention.

In some embodiments the one or more agents in the composition are selected from among:

(a) an agent for treatment of osteopenia selected from among bisphosphonate (e.g., alendronate, risedronate, ibandronate, zoledronic acid), teriparatide, denosumab, and calcitonin; or

(b) an agent for treatment of an autoimmune disorder selected from among a corticosteroid (such as prednisone), nonsteroid drug such as azathioprine, cyclophosphamide, methotrexate, mycophenolate, mofetil, sirolimus, rituximab, tacrolimus, cyclosporine, or other immunosuppressive agent.

The HYD1 peptide used in the methods and compositions of the invention may be a cyclized peptide (cyclic peptide) or a non-cyclic peptide. In some embodiments, the HYD1 peptide is one or more cyclic peptides disclosed in International Publication No. WO 2011/115688 (Hazlehurst et al., “Integrin Interaction Inhibitors for the Treatment of Cancer”, published Sep. 22, 2011), which is incorporated herein by reference in its entirety. The cyclic peptides typically comprise a recognition sequence and a non-recognition sequence, wherein the recognition sequence comprises at least four amino acids (e.g., 5 amino acids), wherein the non-recognition sequence comprises at least four amino acids (e.g., 5 amino acids), and wherein the recognition sequence is joined to the non-recognition sequence by a first linker and a second linker (such as those disclosed in WO 2011/115688 or shown in FIGS. 35-37 herein). In some embodiments, the recognition sequence and/or the non-recognition sequence are in reverse order (i.e., N terminus→carboxy terminus) from those shown in the chemical structures. For example, N*VVAW may be positioned in reverse order as WAVVN*, where N* is norleucine.

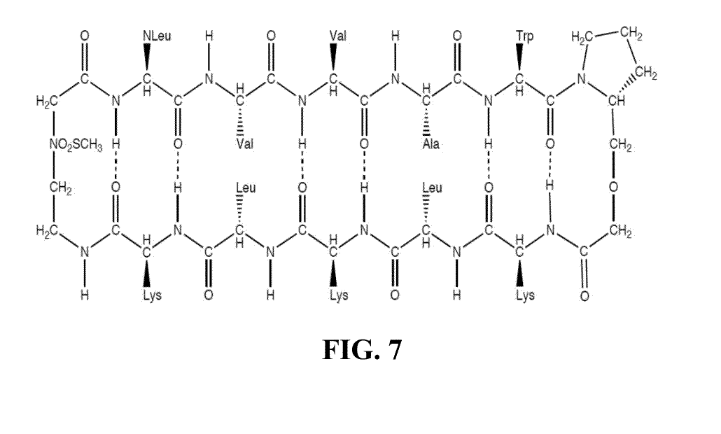

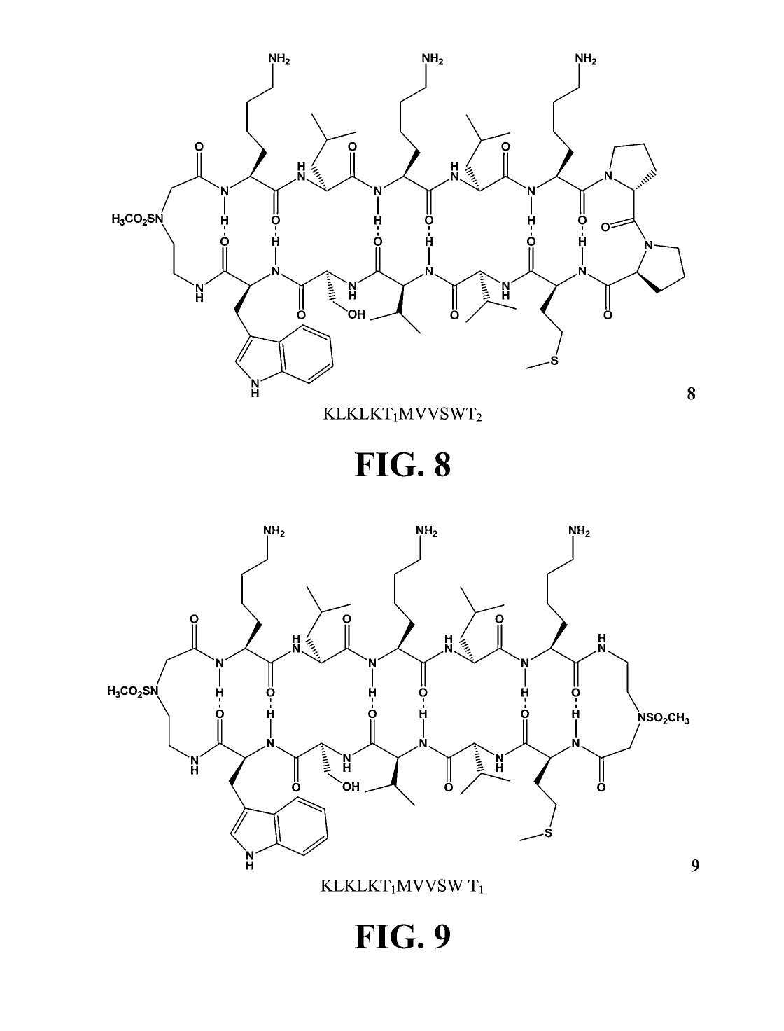

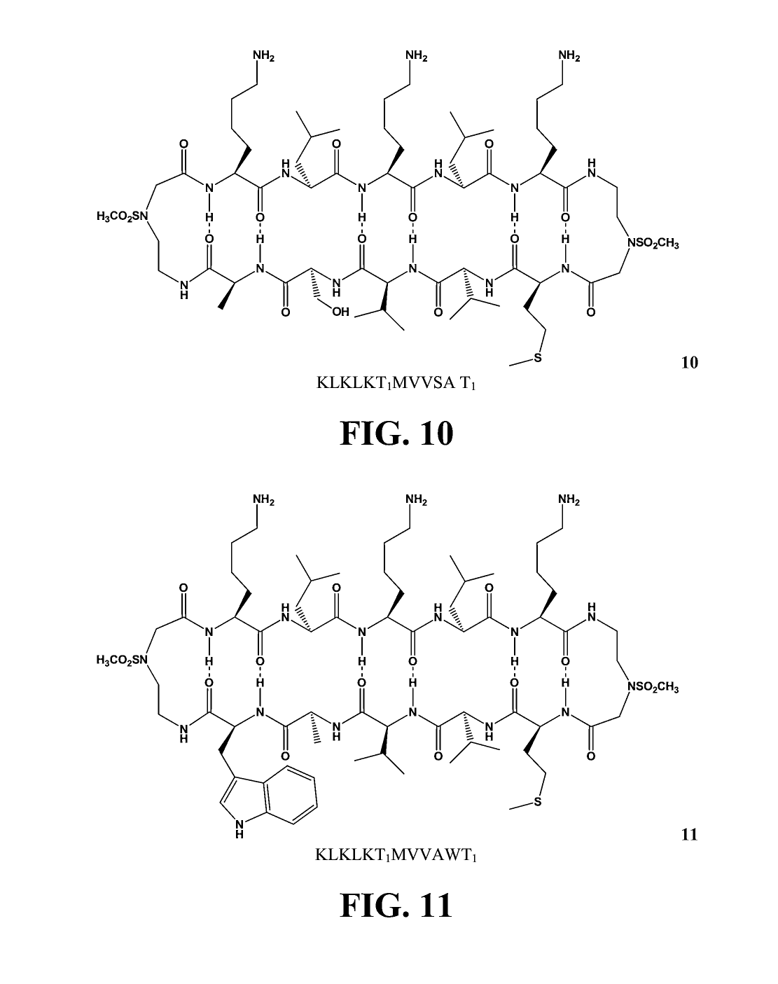

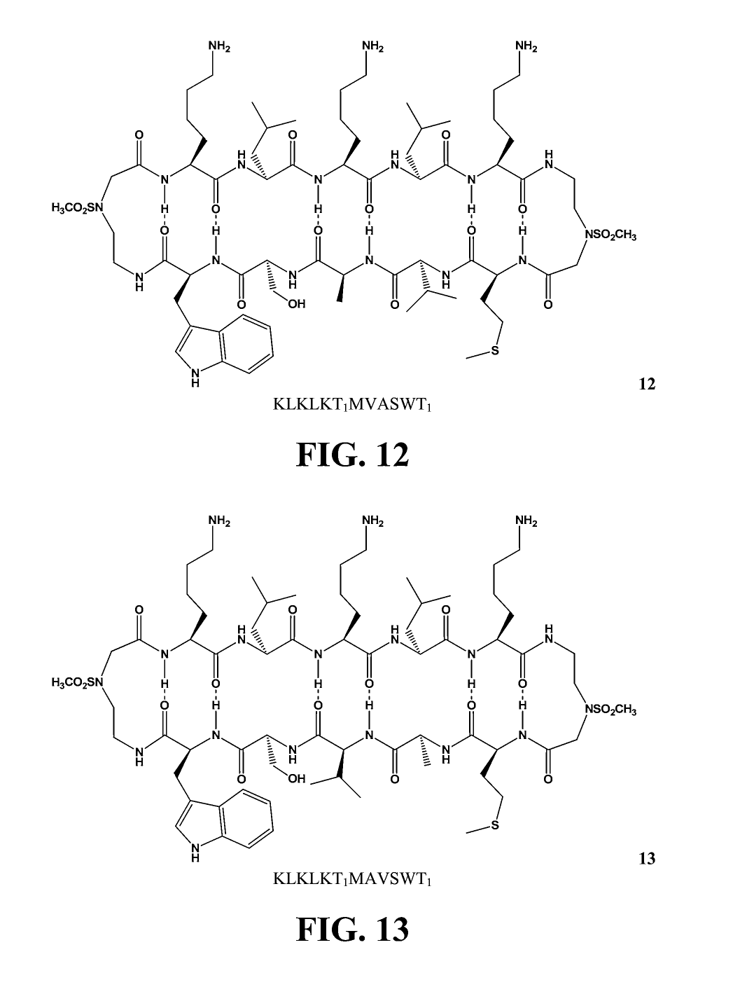

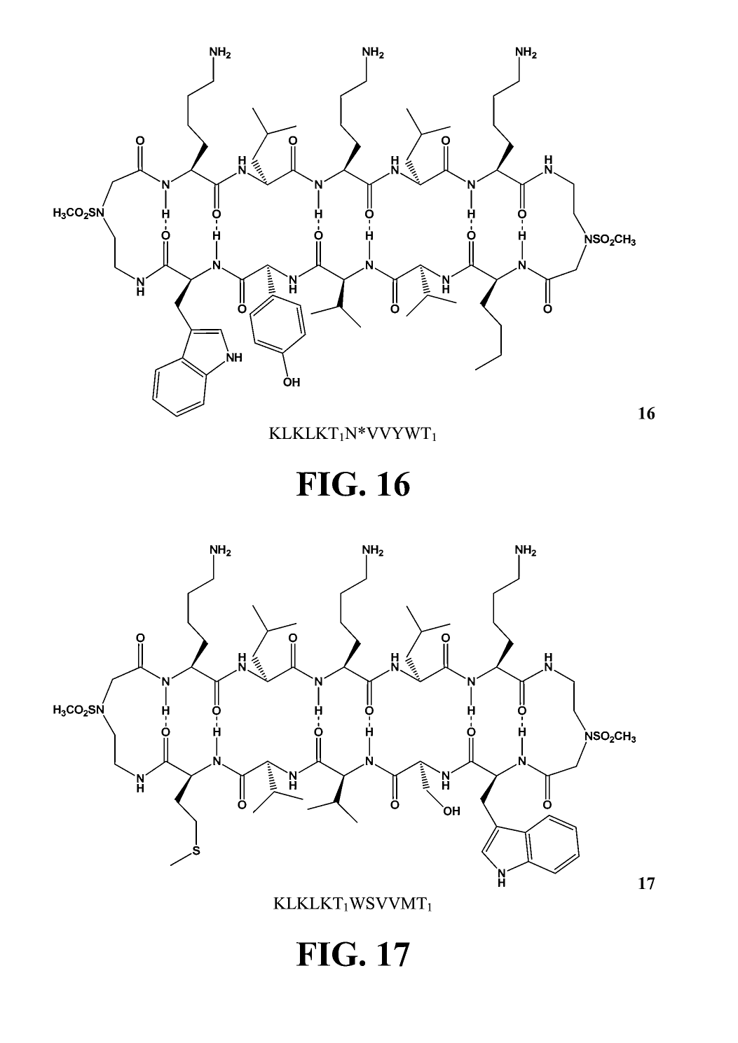

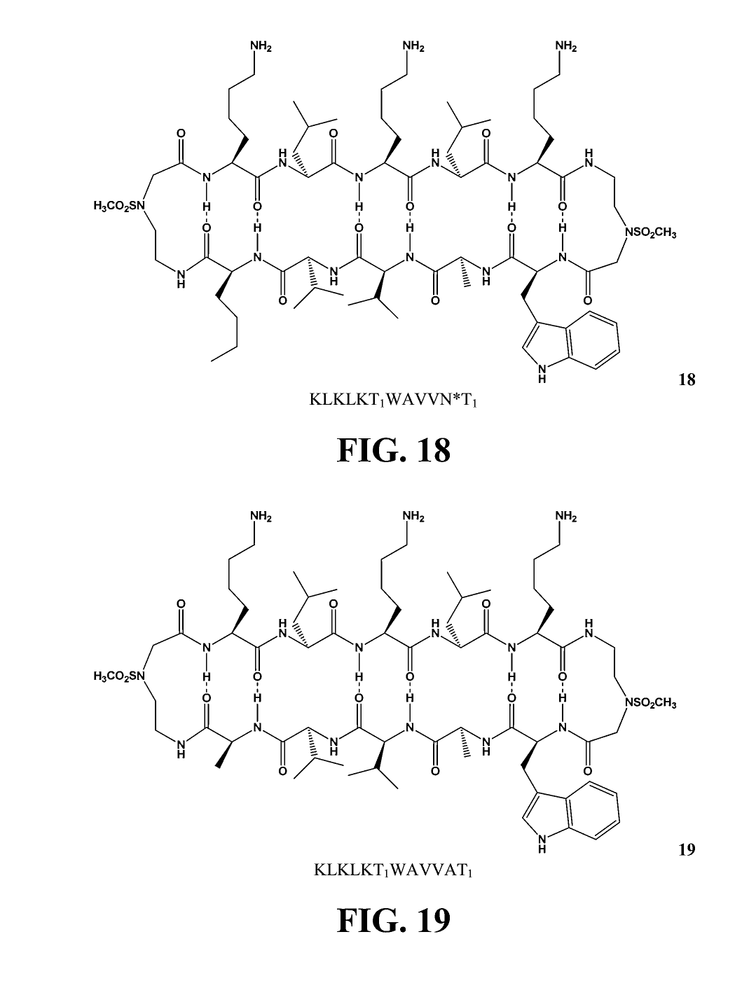

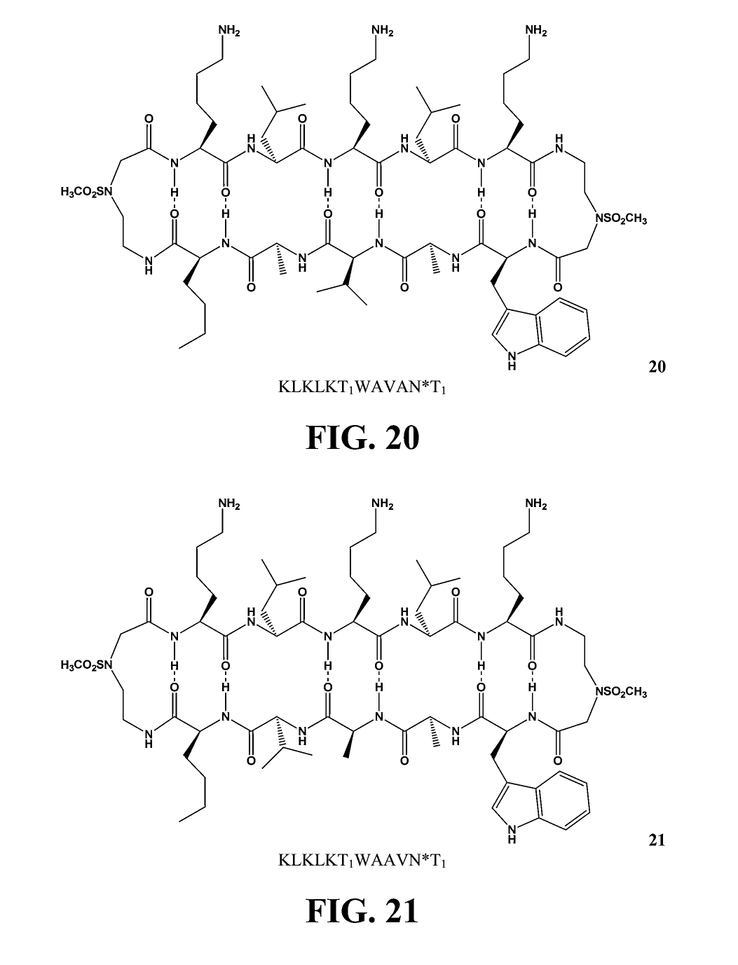

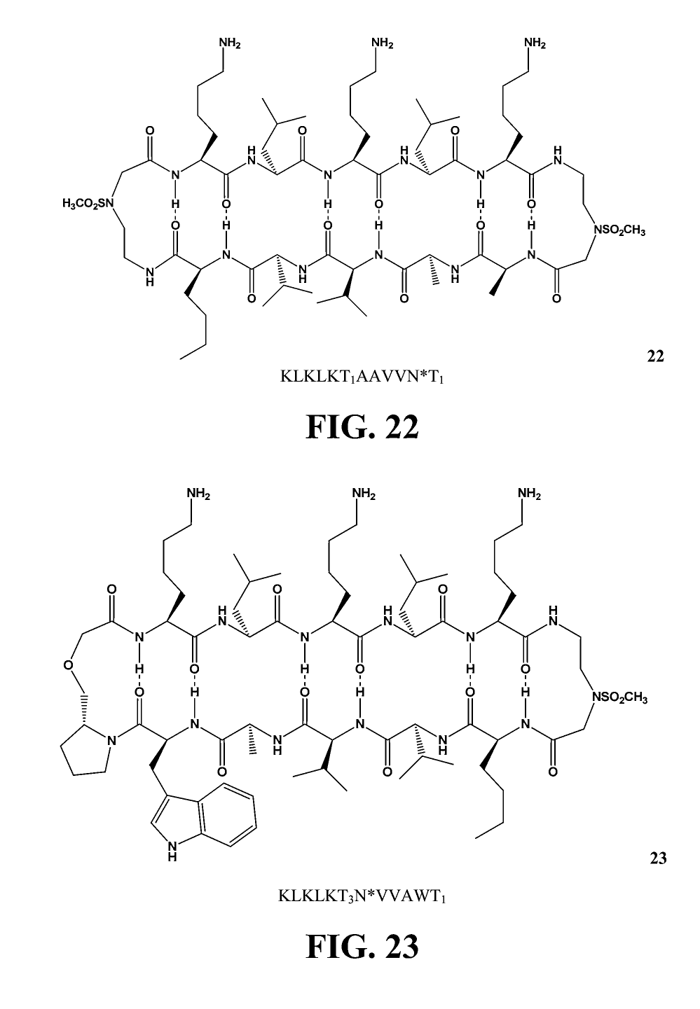

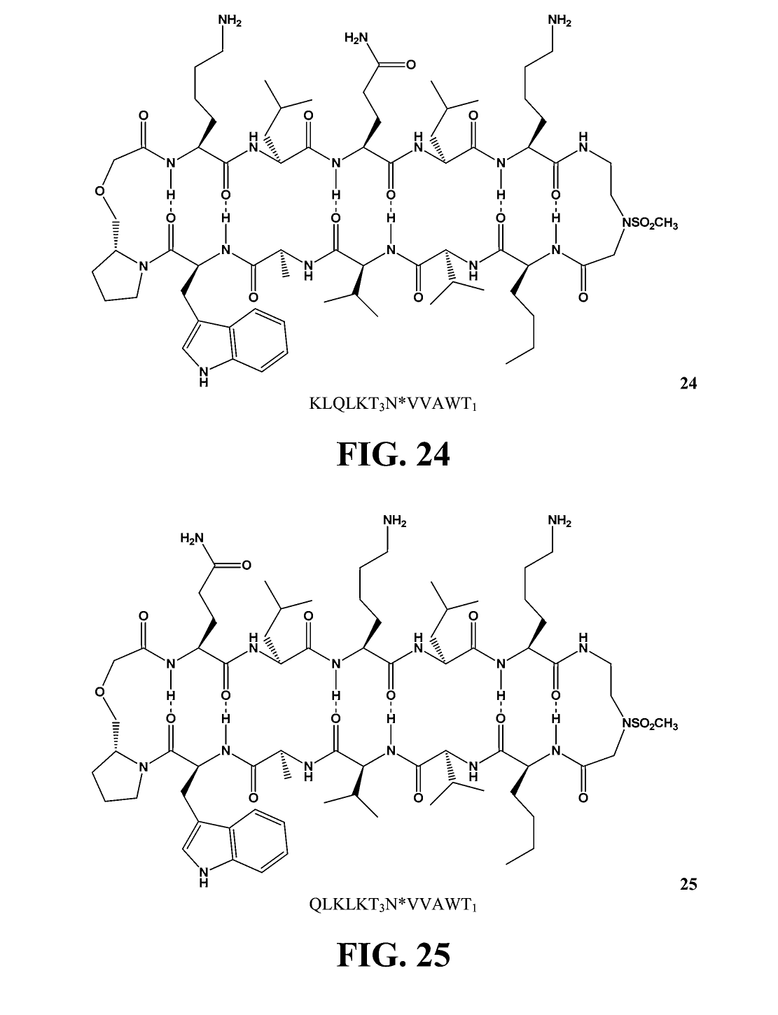

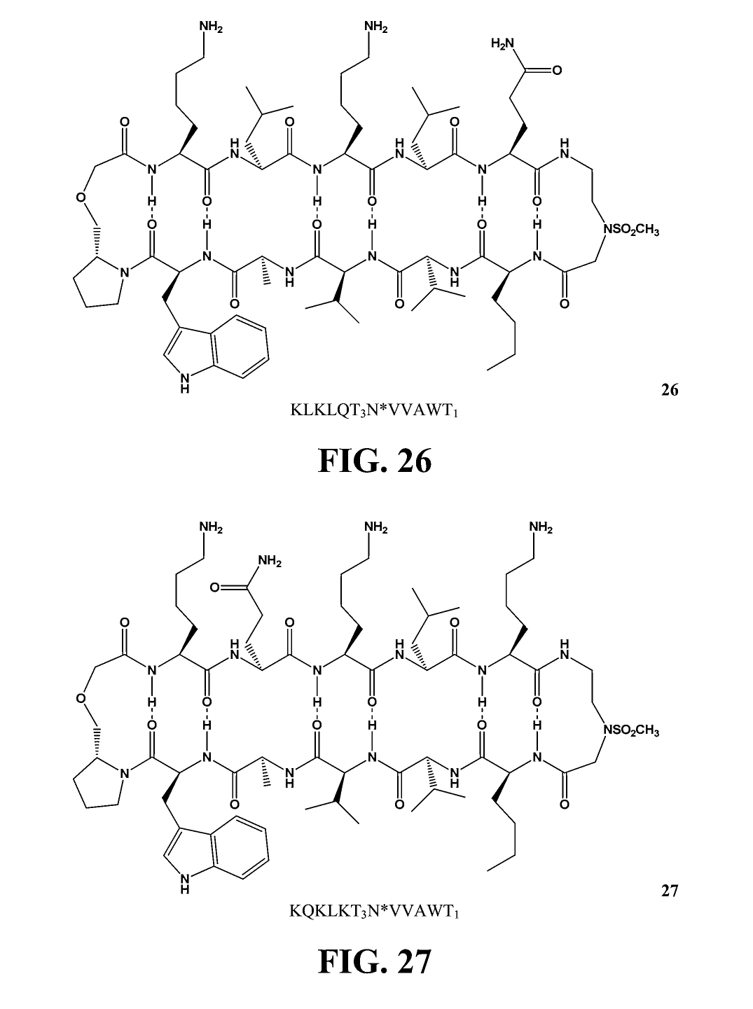

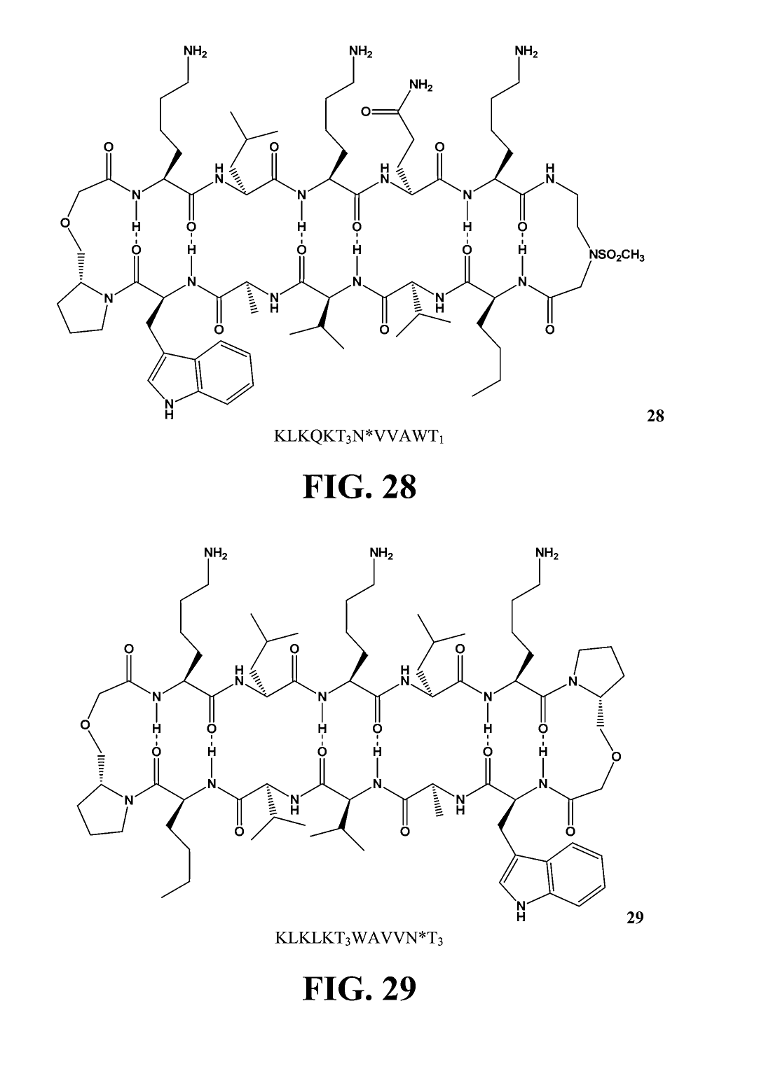

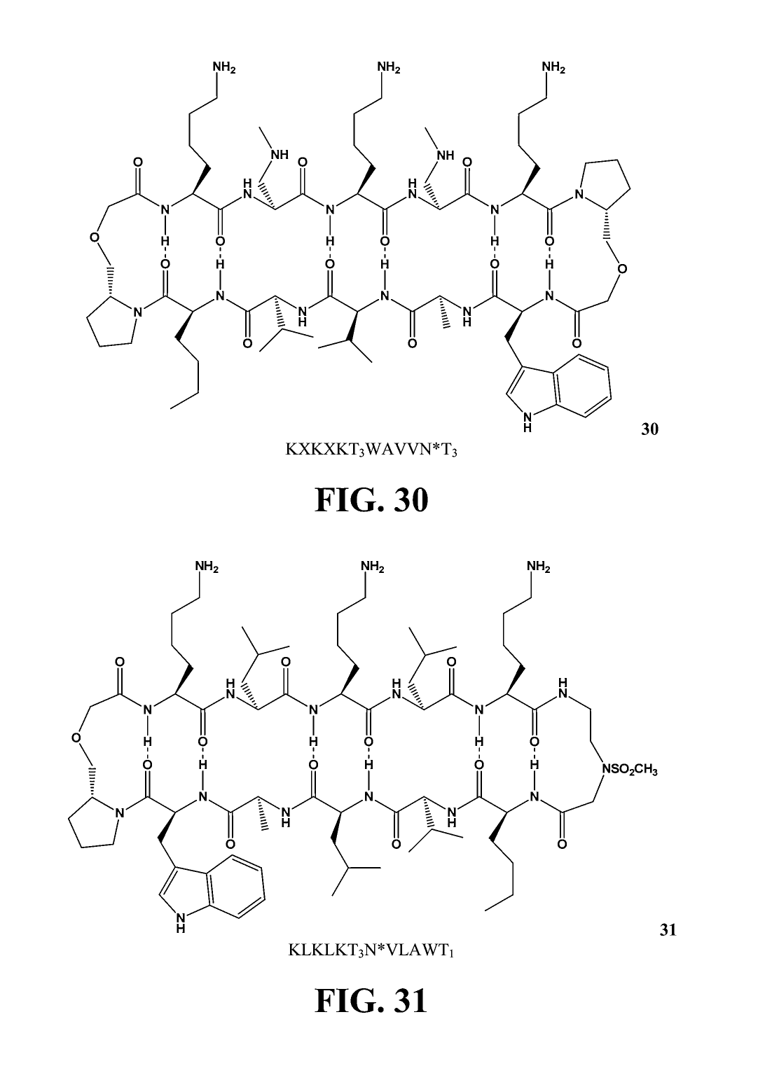

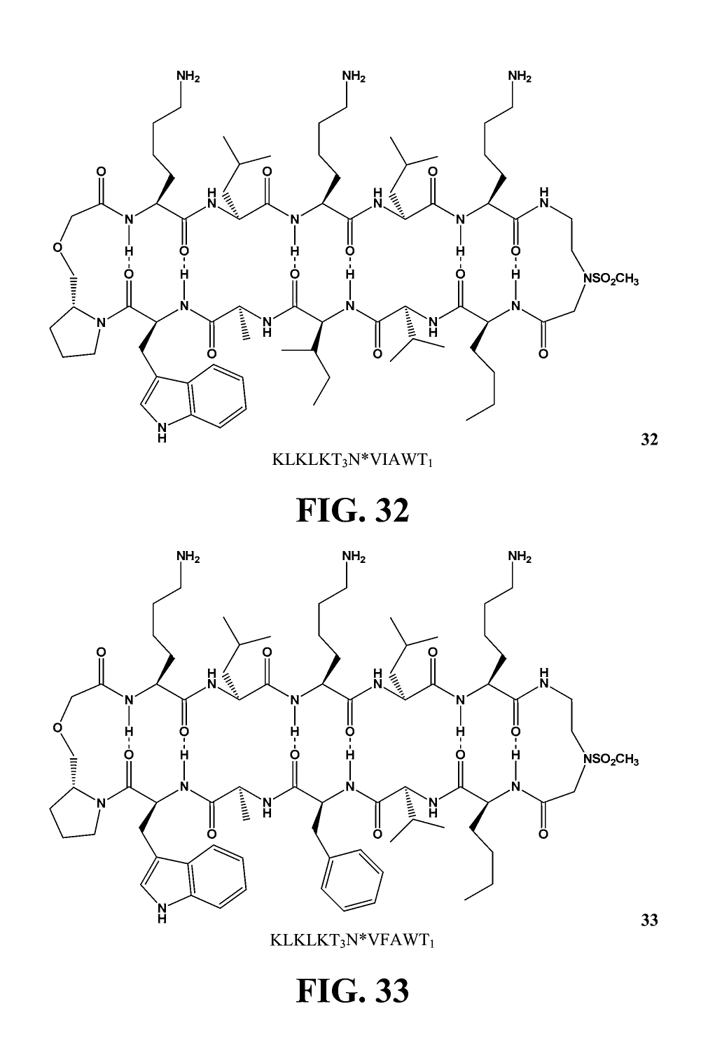

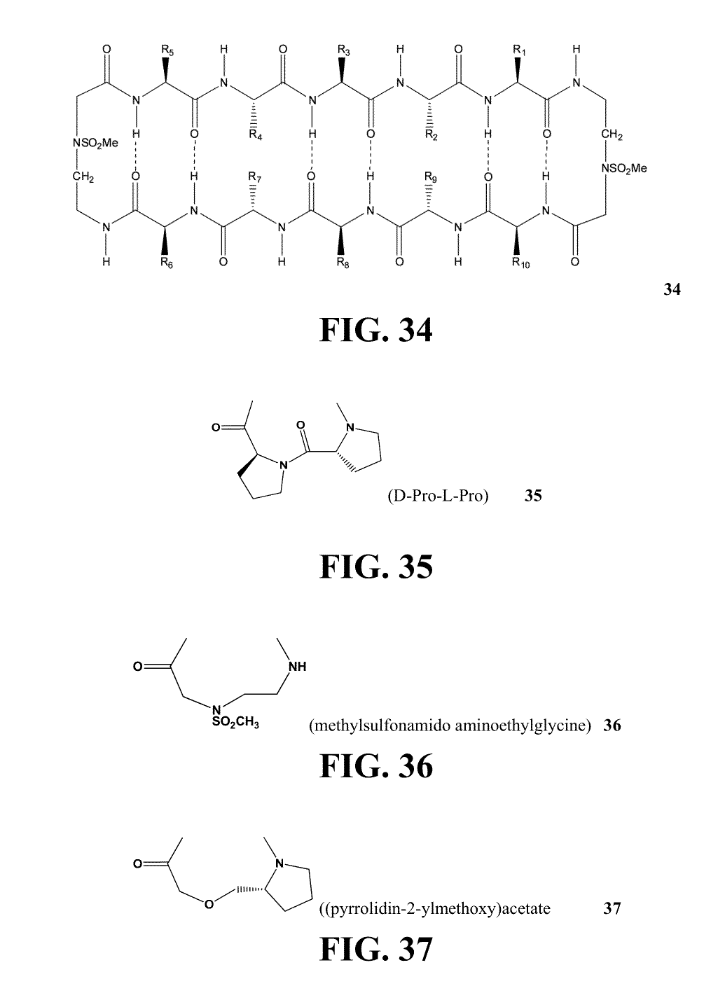

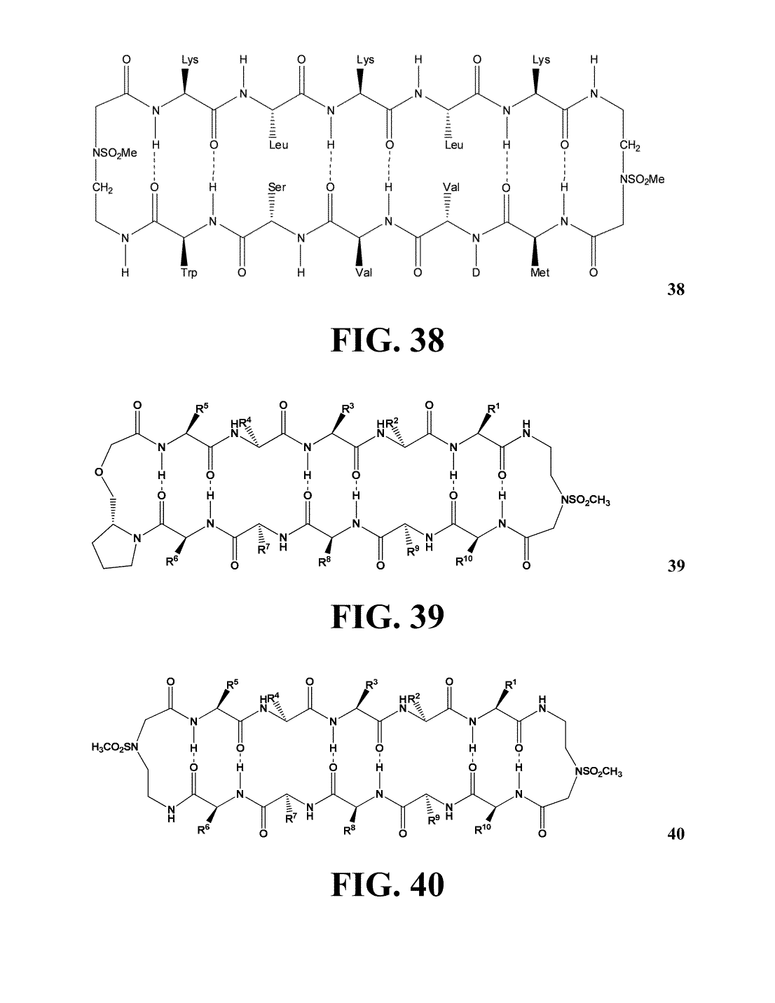

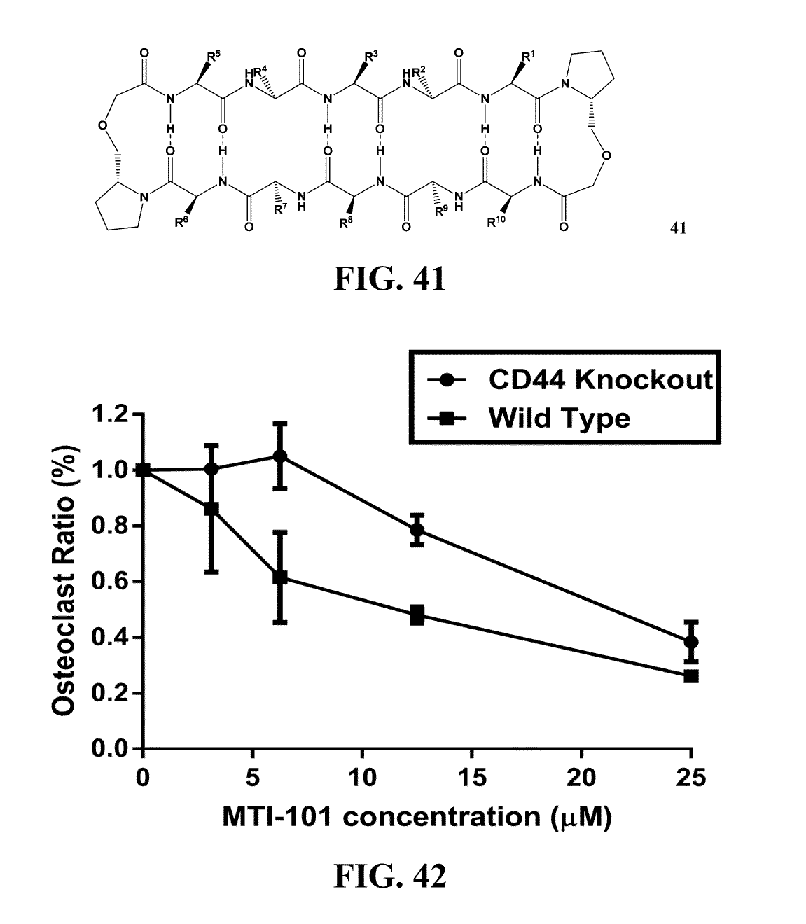

In some embodiments, the cyclic peptide has a chemical structure shown in FIGS. 7-34 or 38-41.

In one embodiment, the cyclic peptide is MTI-101 (FIGS. 7 and 23 herein; compound 16 in Table 8 of WO 2011/115688).

In some embodiments, the cyclic peptide has a chemical structure shown in FIGS. 39-41, wherein R¹ through R⁵ and R⁶ through R¹⁰ are substituents of natural or unnatural amino acids, wherein a sequence of amino acids with R¹ through R⁵ is a non-recognition sequence and a sequence of amino acids with R⁶ through R¹⁰ is a recognition sequence. In some embodiments, the non-recognition sequence is KLKLK, KLQLK, QLKLK, KQKLK, or KXKXK where X=sarcosine where either end of the non-recognition sequence is N-terminus, and wherein the recognition sequence is MVVSW, MVVSA, MVVAW, MVASW, MAVSW, AVVSW, N*VVSW, N*VVYW, N*VVAW, AVVAW, N*AVAW, N*VAAW, N*VVAW, N*VLAW, N*VIAW, N*VFAW, or WSVVW, where N*=norleucine, where either end of the recognition sequence is N-terminus. In some embodiments, the non-recognition sequence is KLKLK, and wherein the recognition sequence is WAVAW, WAVAA, WAVAM, WAVAN*, WAVVN*, WAVSN*, WAVAW, WAAAA, WAAAM, WAAAN*, WAAVW, WAAVA, WAAVM, WAAVN*, WAASN*, WVVAW, WVVAA, WVVAM, WVVAN*, WVVVW, WVVVA, WVVVM, WVVVN*, WVVSN*, WVAVN*, WVAVW, WVAVA, WVAVM, WVAVN*, WVASN*, WSVVW, WSVAA, WSVAM, WSVAN*, WSVVW, WSVVA, WSVVM, WSVVN*, WSVSW, WSVSA, WSVSM, WSVSN*, WSAAW, WSAAA, WSAAM, WSAAN*, WSAVW, WSAVA, WSAVM, WSAVN*, WSASW, WSASA, WSASM, WSASN*, WYVAW, WYVAA, WYVAM, WYVAN*, WYVVW, WYVVA, WYVVM, WYVVN*, WYVSW, WYVSA, WYVSM, WYVSN*, WYAAW, WYAAA, WYAAM, WYAAN*, WYAVW, WYAVA, WYAVM, WYAVN*, WYASW, WYASA, WYASM, WYASN*, AAVAA, AAVAM, AAVAN*, AAVVN*, AAVSN*, AAAAA, AAAAM, AAAAN*, AAAVW, AAAVA, AAAVM, AAAVN*, AAASM, AAASN*, AVVAW, AVVAA, AVVAM, AVVAN*, AVVVA, AVVVM, AVVVN*, AVVSN*, AVAAW, AVAAM, AVAAN*, AVAVA, AVAVM, AVAVN*, AVASN*, ASVAW, ASVAA, ASVAM, ASVAN*, ASVVW, ASVVA, ASVVM, ASVVN*, ASVSA, ASVSM, ASVSN*, ASAAW, ASAAA, ASAAM, ASAAN*, ASAVW, ASAVA, ASAVM, ASAVN*, ASASA, ASASM, ASASN*, AYVAW, AYVAA, AYVAM, AYVAN*, AYVVW, AYVVA, AYVVM, AYVVN*, AYVSW, AYVSA, AYVSM, AYVSN*, AYAAW, AYAAA, AYAAM, AYAAN*, AYAVW, AYAVA, AYAVM, AYAVN*, AYASW, AYASA, AYASM, AYASN*, MAVAA, MAVAM, MAVAN*, MAVVN*, MAVSN*, MAAAA, MAAAM, MAAAN*, MAAVW, MAAVA, MAAVM, MAAVN*, MAASN*, MVVAW, MVVAA, MVVAM, MVVAN*, MVVVM, MVVVN*, MVVSN*, MVAAM, MVAAN*, MVAVM, MVAVN*, MVASN*, MSVAW, MSVAA, MSVAM, MSVAN*, MSVVW, MSVVA, MSVVM, MSVVN*, MSVSM, MSVSN*, MSAAW, MSAAA, MSAAM, MSAAN*, MSAVW, MSAVA, MSAVM, MSAVN*, MSASM, MSASN*, MYVAW, MYVAA, MYVAM, MYVAN*, MYVVW, MYVVA, MYVVM, MYVVN*, MYVSW, MYVSA, MYVSM, MYVSN*, MYAAW, MYAAA, MYAAM, MYAAN*, MYAVW, MYAVA, MYAVM, MYAVN*, MYASW, MYASA, MYASM, or MYASN*, where N*=norleucine, where either end of the recognition sequence is N-terminus.

In some embodiments, the cyclic peptide is one in Table 4, 5, or 8 of International Publication No. WO 2011/115688, which is hereby incorporated herein by reference in its entirety.

In some embodiments, the recognition sequence and/or the non-recognition sequence are in reverse order (i.e., N terminus→carboxy terminus) from those shown in the chemical structures. For example, N*VVAW may be positioned in reverse order as WAVVN*, where N* is norleucine.

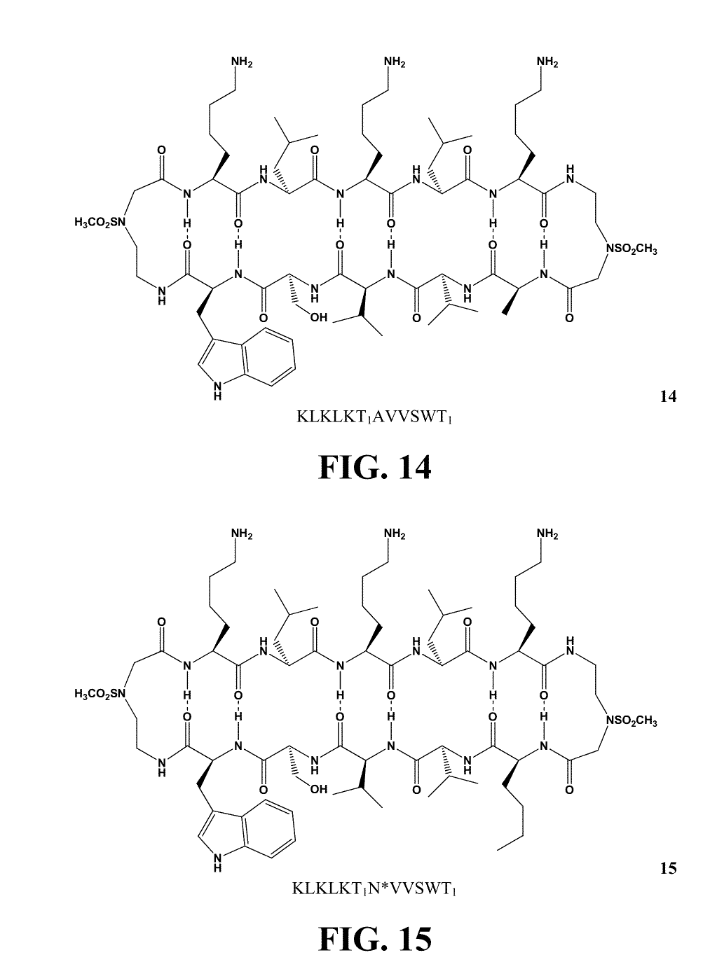

In some embodiments, the HYD1 peptide is one or more peptides disclosed in U.S. Pat. No. 7,632,814 (Hazlehurst et al., “HYD1 peptides as anti-cancer agents”), which is incorporated herein by reference in its entirety. In some embodiments, the HYD1 peptide is one or more peptides selected from among KIKMVISWKG (HYD1; SEQ ID NO:1); AIAMVISWAG (SEQ ID NO:2; HYD8); AIKMVISWAG (SEQ ID NO:3; HYDE); AIKMVISWKG (SEQ ID NO:4; HYD2); AKMVISW (SEQ ID NO:5); AKMVISWKG (SEQ ID NO:6); IAMVISW (SEQ ID NO:7); IAMVISWKG (SEQ ID NO:8); IKAVISW (SEQ ID NO:9); IKAVISWKG (SEQ ID NO:10); IKMAISW (SEQ ID NO:11); IKMAISWKG (SEQ ID NO:12); IKMVASW (SEQ ID NO:13); IKMVASWKG (SEQ ID NO:14); IKMVIAW (SEQ ID NO:15); IKMVIAWKG (SEQ ID NO:16); IKMVISA (SEQ ID NO:17); IKMVISWKG (SEQ ID NO:18); IKMVISW (SEQ ID NO:19); IKMVISWAG (SEQ ID NO:20); KMVISWKA (SEQ ID NO:21); IKMVISWKG (SEQ ID NO:22; HYD18; (-K)HYD1); ISWKG (SEQ ID NO:23); KAKMVISWKG (SEQ ID NO:24); KIAMVISWAG (SEQ ID NO:25; HYD7); KIAMVISWKG (SEQ ID NO:26); KIKAVISWKG (SEQ ID NO:27); KIKMAISWKG (SEQ ID NO:28); KIKMV (SEQ ID NO:29); KIKMVASWKG (SEQ ID NO:30); KIKMVI (SEQ ID NO:31; HYD16); KIKMVIAWKG (SEQ ID NO:32); KIKMVIS (SEQ ID NO:33; HYD15); KIKMVISAKG (SEQ ID NO:34); KIKMVISW (SEQ ID NO:35; HYD14); KIKMVISWAG (SEQ ID NO:36); KIKMVISWK (SEQ ID NO:37; HYD17; HYD1(-G)); KIKMVISWKA (SEQ ID NO:38); KMVISWKG (SEQ ID NO:39; HYD9); LSWKG (SEQ ID NO:40; HYD12); MVISWKG (SEQ ID NO:41; HYD10); SWKG (SEQ ID NO:42; HYD13); VISWKG (SEQ ID NO:43; HYD11); WIKSMKIVKG (SEQ ID NO:44); KMVIXW (SEQ ID NO:45); IKMVISWXX (SEQ ID NO:46); and KMVISWXX (SEQ ID NO:47); wherein X is any amino acid (traditional or non-traditional amino acid). In another embodiment, the peptide consists of the amino acid sequence. In another embodiment, the peptide consists essentially of the amino acid sequence. In another embodiment, the peptide is one listed in the figures in U.S. Pat. No. 7,632,814. In one embodiment, the peptide is one of the variants listed in FIGS. 14A-14C, 15A-15C, or 16A-1-16C-2 of U.S. Pat. No. 7,632,814 and is substituted with an alanine at one position, and wherein another residue is substituted in place of the alanine.

In some embodiments, the peptide comprises at least one D-amino acid. In some embodiments, each amino acid of the peptide is a D-amino acid.

In some embodiments, the peptide is a non-cyclic peptide and the peptide is administered to the subject as a nucleic acid molecule encoding the peptide. The nucleic acid may be administered within a vector (viral or non-viral) and may include any necessary regulatory sequence(s) (e.g., a promoter) for expression of the peptide in vivo. Likewise, compositions of the invention may comprise a nucleic acid encoding the peptide or a vector comprising the nucleic acid sequence with any necessary regulatory sequence.

In the various cyclic and non-cyclic HYD1 peptides, one or more one or more amino acid residues or substituents within the sequence can be substituted by another amino acid of a similar polarity which acts as a functional equivalent, resulting in a silent alteration. Conservative substitutions for an amino acid within the sequence may be selected from other members of the class to which the amino acid belongs (see Table 1). Conservative substitutions also include substitutions by amino acids having chemically modified side chains that do not eliminate the biological function of the resulting variant.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

The bone deficiency may be osteopenia, bone lesion, or other condition involving bone loss. In some embodiments, the bone deficiency is caused by an osteopenic disorder. Examples of osteopenic disorders that may be treated include osteoporosis, Paget's disease, lytic bone metastases, periodontitis, rheumatoid arthritis, and bone loss due to immobilization.

In some embodiments, the subject has a cancer that increases osteoclast activity and/or induces bone resorption. In some embodiments, the subject does not have cancer.

In some embodiments, the subject has an autoimmune disorder. Examples of autoimmune disorders that may be treated include AIDS-associated myopathy, AIDS-associated neuropathy, Acute disseminated encephalomyelitis, Addison's Disease, Alopecia Areata, Anaphylaxis Reactions, Ankylosing Spondylitis, Antibody-related Neuropathies, Antiphospholipid Syndrome, Arthritis (e.g., rheumatoid arthritis, psoriatic arthritis, juvenile idiopathic arthritis), Autism, Autoimmune Atherosclerosis, Autoimmune Diabetes Insipidus, Autoimmune Endometriosis, Autoimmune Eye Diseases, Autoimmune Gastritis, Autoimmune Hemolytic Anemia, Autoimmune Hemophilia, Auto immune Hepatitis, Auto immune Interstitial Cystitis, Auto immune Lymphoproliferative Syndrome, Autoimmune Myelopathy, Autoimmune Myocarditis, Autoimmune Neuropathies, Autoimmune Oophoritis, Autoimmune Orchitis, Autoimmune Thrombocytopenia, Autoimmune Thyroid Diseases, Autoimmune Urticaria, Autoimmune Uveitis, Autoimmune Vasculitis, Behcet's Disease, Bell's Palsy, Bullous Pemphigoid, CREST, Celiac Disease, Cerebellar degeneration (paraneoplastic), Chronic Fatigue Syndrome, Chronic Rhinosinusitis, Chronic inflammatory demyelinating polyneuropathy, Churg Strauss Syndrome, Connective Tissue Diseases, Crohn's Disease, Cutaneous Lupus, Dermatitis Herpetiformis, Dermatomyositis, Diabetes Mellitus, Discoid Lupus Erythematosus, Drug-induced Lupus, Endocrine Orbitopathy, Glomerulonephritis, Goodpasture Syndrome, Goodpasture's Syndrome, Graft-versus-Host Disease (GVHD), Graves Disease, Guillian-Barre Syndrome, Miller Fisher variant of the Guillian Barre Syndrome, axonal Guillian Barre Syndrome, demyelinating Guillian Bane Syndrome, Hashimoto Thyroiditis, Herpes Gestationis, Human T-cell lymphomavirus-associated myelopathy, Huntington's Disease, IgA Nephropathy, Immune Thrombocytopenic Purpura, Inclusion body myositis, Interstitial Cystitis, Isaacs syndrome, Lambert Eaton myasthenic syndrome, Limbic encephalitis, Lower motor neuron disease, Lyme Disease, MCTD, Microscopic Polyangiitis, Miller Fisher Syndrome, Mixed Connective Tissue Disease, Mononeuritis multiplex (vasculitis), Multiple Sclerosis (relapsing-remitting MS (RRMS), secondary-progressive MS (SPMS), primary-progressive MS (PPMS), and progressive-relapsing MS (PRMS)), Myasthenia Gravis, Myxedema, Meniere Disease, Neonatal LE, Neuropathies with dysproteinemias, Opsoclonus-myoclonus, PBC, POEMS syndrome, Paraneoplastic Autoimmune Syndromes, Pemphigus, Pemphigus Foliaceus, Pemphigus Vulgaris, Pernicious Anemia, Peyronie's Disease, Plaque Psoriasis, Plasmacytoma/myeloma neuropathy, Poly-Dermatomyositis, Polyarteritis Nodosa, Polyendocrine Deficiency Syndrome, Polyendocrine Deficiency Syndrome Type 1, Polyendocrine Deficiency Syndrome Type 2, Polyglandular Autoimmune Syndrome Type I, Polyglandular Autoimmune Syndrome Type II, Polyglandular Autoimmune Syndrome Type III, Polymyositis, Primary Biliary Cirrhosis, Primary Glomerulonephritis, Primary Sclerosing Cholangitis, Psoriasis, Psoriatic Arthritis, Rasmussen's Encephalitis, Raynaud's Disease, Relapsing Polychondritis, Retrobulbar neuritis, Rheumatic Diseases, Rheumatoid Arthritis, Scleroderma, Sensory neuropathies (paraneoplastic), Sjogren's Syndrome, Stiff-Person Syndrome, Subacute Thyroiditis, Subacute autonomic neuropathy, Sydenham Chorea, Sympathetic Ophthalmitis, Systemic Lupus Erythematosus, Transverse myelitis, Type 1 Diabetes, Ulcerative Colitis, Vasculitis, Vitiligo, Wegener's Granulomatosis, acrocyanosis, anaphylacetic reaction, autoimmune inner ear disease, bilateral sensorineural hearing loss, cold agglutinin hemolytic anemia, cold-induced immune hemolytic anemia, idiopathic endolymphatic hydrops, idiopathic progressive bilateral sensorineural hearing loss, immune-mediated inner ear disease, and mixed autoimmune hemolysis.

In some embodiments, the peptide is administered before, during, and/or after a transplant to delay the onset of GVHD. The transplant may be, for example, an xenograft or an allograft, such as an allogeneic stem cell, bone marrow, or organ transplant.

The method may further comprise administration of one or more agents for treatment of the bone deficiency and/or autoimmune disorder or for treatment of another disorder before, during, and/or after administration of the peptide. The agent(s) may be administered in a the same formulation as the peptide(s) or in a separate formulation(s).

In some embodiments, the method comprises administration of the one or more of the following before, during, and/or after administration of the peptide:

(a) an agent for treatment of bone deficiency selected from among bisphosphonate (e.g., alendronate, risedronate, ibandronate, zoledronic acid), teriparatide, denosumab, and calcitonin; or

(b) an agent for treatment of an autoimmune disorder selected from among a corticosteroid (such as prednisone), nonsteroid drug such as azathioprine, cyclophosphamide, methotrexate, mycophenolate, mofetil, sirolimus, rituximab, tacrolimus, cyclosporine, or other immunosuppressive agent.

In various embodiments, methods and compositions disclosed herein can ameliorate or inhibit the progression of a bone deficiency such as osteoporosis and/or an autoimmune disorder. In various embodiments, methods and compositions disclosed herein can be used to prevent the occurrence, or delay the onset of, a degenerative bone disease such as, for example, osteoporosis or inflammatory osteolysis. In some embodiments, the methods comprise administering to a subject having a degenerative bone disease a therapeutically effective amount of a HYD1 peptide.

As used herein, unless specified, “a HYD1 peptide” or “a peptide” is inclusive of the d-amino acid peptide having the sequence: KIKMVISWKG (HYD1), as well as other HYD1-related peptides (which includes d-amino acid containing peptides and non-d-amino acid containing peptides) disclosed in U.S. Pat. No. 7,632,814 (Hazelhurst et al., “HYD1 Peptides as Anti-Cancer Agents”), which is incorporated herein by reference in its entirety. As used herein, reference to c-HYD1, C-HYD1 refers to a cyclized or cyclic peptide of the invention.

### Bone Deficiency

The method of the invention includes treatment of a bone deficiency such as osteoporosis. The peptide may be administered with one or more other agents to treat the bone deficiency, such as a bone morphogenic factor, transforming growth factor-beta (TGF-beta), parathyroid hormone or analog thereof, parathyroid hormone related protein or analog thereof, prostaglandin, bisphosphonate, alendronate, fluoride, calcium, fibroblast growth factor (FGF), and FGF modulator.

In some embodiments, the method includes administration of one or more agents selected from among bisphosphonate (e.g., alendronate, risedronate, ibandronate, zoledronic acid), teriparatide, denosumab, and calcitonin.

The bone deficiency may be caused by cancer, an inflammatory condition, or an autoimmune disorder, for example.

In some embodiments the bone deficiency is caused by or otherwise associated with an inflammatory condition and the method further comprises administration of at least one additional therapeutic agent selected from an interleukin-1 (IL-1) inhibitor, IL-1ra, anakinra, a TNF-alpha inhibitor, a soluble TN-alpha receptor, etanercept, an anti-TNF-alpha antibody, infliximab, a D2E7 antibody, a non-steroidal anti-inflammatory drug (NSAID), a COX-2 inhibitor, celecoxib, rofecoxib, and leflunomide.

In some embodiments, the bone deficiency is caused by or otherwise associated with an autoimmune disorder and the method further comprises administering at least one additional therapeutic agent selected from an interleukin-1 (IL-1) inhibitor, IL-1ra, anakinra, a TNF-alpha inhibitor, a soluble TNF-alpha receptor, etanercept, an anti-TNF-alpha antibody, infliximab, a D2E7 antibody, methotrexate, a soluble form of CTLA4, and a modulator of glucocorticoid receptor.

In some embodiments, the bone deficiency is caused by or is otherwise associated with rheumatoid arthritis, and the method further comprises administering at least one additional therapeutic agent selected from an interleukin-1 (IL-1) inhibitor, IL-1ra, anakinra, a TNF-alpha inhibitor, a soluble TNF-alpha receptor, etanercept, an anti-TNF-alpha antibody, infliximab, a D2E7 antibody, a non-steroidal anti-inflammatory drug (NSAID), a COX-2 inhibitor, celecoxib, rofecoxib, leflunomide, methotrexate, a soluble form of CTLA4, and a modulator of glucocorticoid receptor.

In some embodiments, the bone deficiency is caused by or otherwise associated with cancer, and the method further comprises administering at least one additional therapeutic agent selected from a chemotherapeutic drug, keratinocyte growth factor (KGF), a KGF-related molecule, a KGF modulator, an anti-Her2 antibody, an anti-CDC20 antibody, and an anti-EGFR antibody, and a PAF antagonist.

In some embodiments, the bone deficiency is caused by or is otherwise associated with a proliferation disorder, such as cancer, and the method further comprises administering radiation therapy and/or chemotherapy.

In some embodiments, the bone deficiency is caused by or otherwise associated with cancer selected from at least one of breast cancer, prostate cancer, thyroid cancer, kidney cancer, lung cancer, esophageal cancer, rectal cancer, bladder cancer, cervical cancer, ovarian cancer, and liver cancer, and gastrointestinal tract cancer.

In some embodiments, the bone deficiency is caused by or otherwise associated with a solid tumor.

In some embodiments, the bone deficiency is caused by or is associated with a metastatic tumor.

In some embodiments, the bone deficiency is not caused by or otherwise associated with cancer.

In some embodiments, the bone deficiency is caused by or otherwise associated with at least one condition selected from osteoporosis, Paget's disease, osteomyelitis, hypercalcemia, osteopenia, and osteonecrosis.

The subject may have an osteopenic disorder. As used herein, the term “osteopenic disorder” includes, but is not limited to, osteoporosis, osteopenia, Paget's disease, lytic bone metastases, periodontitis, rheumatoid arthritis, and bone loss due to immobilization. In addition to these bone disorders, certain cancers are known to increase osteoclast activity and induce bone resorption, such as breast, prostate, and multiple myeloma. The osteopenic disorder may be chronic or acute.

Conditions that may be treated according to certain embodiments include, but are not limited to, the following: osteoporosis, including, but not limited to, primary osteoporosis, endocrine osteoporosis (including, but not limited to, hyperthyroidism, hyperparathyroidism, Cushing's syndrome, and acromegaly), hereditary and congenital forms of osteoporosis (including, but not limited to, osteogenesis imperfecta, homocystinuria, Menkes' syndrome, Riley-Day syndrome), and osteoporosis due to immobilization of extremities; Paget's disease of bone (osteitis deformans) in adults and juveniles; Osteomyelitis, i.e., an infectious lesion in bone, leading to bone loss; Hypercalcemia, including, but not limited to, hypercalcemia resulting from solid tumors (including, but not limited to, breast, lung and kidney) and hematologic malignacies (including, but not limited to, multiple myeloma, lymphoma and leukemia), idiopathic hypercalcemia, and hypercalcemia associated with hyperthyroidism and renal function disorders; Osteopenia, including but not limited to, osteopenia following surgery, osteopenia induced by steroid administration, osteopenia associated with disorders of the small and large intestine, and osteopenia associated with chronic hepatic and renal diseases; Osteonecrosis, i.e., bone cell death, including, but not limited to, osteonecrosis associated with traumatic injury, osteonecrosis associated with Gaucher's disease, osteonecrosis associated with sickle cell anemia, osteonecrosis associated with systemic lupus erythematosus, osteonecrosis associated with rheumatoid arthritis, osteonecrosis associated with periodontal disease, osteonecrosis associated with osteolytic metastasis, and osteonecrosis associated with other condition; and loss of cartilage and joint erosion associated with rheumatoid arthritis.

### Autoimmune Disorders

Autoimmune disorders are diseases caused by an immune response against the body's own cells or tissues. Autoimmune disorders result in destruction of one or more types of body tissues, abnormal growth of an organ or organs, or changes in organ function or functions. The disorders may affect only one organ or tissue type or may affect multiple organs and tissue types. In addition, a person may experience one or more autoimmune disorders at the same time. Organs and tissues commonly affected by autoimmune disorders include blood components such as red blood cells, blood vessels, connective tissues, endocrine glands such as the thyroid or pancreas, muscles, joints, and skin.

Examples of autoimmune disorders that may be treated include AIDS-associated myopathy, AIDS-associated neuropathy, Acute disseminated encephalomyelitis, Addison's Disease, Alopecia Areata, Anaphylaxis Reactions, Ankylosing Spondylitis, Antibody-related Neuropathies, Antiphospholipid Syndrome, Arthritis (e.g., rheumatoid arthritis, psoriatic arthritis, juvenile idiopathic arthritis), Autism, Autoimmune Atherosclerosis, Autoimmune Diabetes Insipidus, Autoimmune Endometriosis, Autoimmune Eye Diseases, Autoimmune Gastritis, Autoimmune Hemolytic Anemia, Autoimmune Hemophilia, Auto immune Hepatitis, Auto immune Interstitial Cystitis, Auto immune Lymphoproliferative Syndrome, Autoimmune Myelopathy, Autoimmune Myocarditis, Autoimmune Neuropathies, Autoimmune Oophoritis, Autoimmune Orchitis, Autoimmune Thrombocytopenia, Autoimmune Thyroid Diseases, Autoimmune Urticaria, Autoimmune Uveitis, Autoimmune Vasculitis, Behcet's Disease, Bell's Palsy, Bullous Pemphigoid, CREST, Celiac Disease, Cerebellar degeneration (paraneoplastic), Chronic Fatigue Syndrome, Chronic Rhinosinusitis, Chronic inflammatory demyelinating polyneuropathy, Churg Strauss Syndrome, Connective Tissue Diseases, Crohn's Disease, Cutaneous Lupus, Dermatitis Herpetiformis, Dermatomyositis, Diabetes Mellitus, Discoid Lupus Erythematosus, Drug-induced Lupus, Endocrine Orbitopathy, Glomerulonephritis, Goodpasture Syndrome, Goodpasture's Syndrome, Graft-versus-Host Disease (GVHD), Graves Disease, Guillian-Barre Syndrome, Miller Fisher variant of the Guillian Bane Syndrome, axonal Guillian Barre Syndrome, demyelinating Guillian Bane Syndrome, Hashimoto Thyroiditis, Herpes Gestationis, Human T-cell lymphomavirus-associated myelopathy, Huntington's Disease, IgA Nephropathy, Immune Thrombocytopenic Purpura, Inclusion body myositis, Interstitial Cystitis, Isaacs syndrome, Lambert Eaton myasthenic syndrome, Limbic encephalitis, Lower motor neuron disease, Lyme Disease, MCTD, Microscopic Polyangiitis, Miller Fisher Syndrome, Mixed Connective Tissue Disease, Mononeuritis multiplex (vasculitis), Multiple Sclerosis (relapsing-remitting MS (RRMS), secondary-progressive MS (SPMS), primary-progressive MS (PPMS), and progressive-relapsing MS (PRMS)), Myasthenia Gravis, Myxedema, Meniere Disease, Neonatal LE, Neuropathies with dysproteinemias, Opsoclonus-myoclonus, PBC, POEMS syndrome, Paraneoplastic Autoimmune Syndromes, Pemphigus, Pemphigus Foliaceus, Pemphigus Vulgaris, Pernicious Anemia, Peyronie's Disease, Plaque Psoriasis, Plasmacytoma/myeloma neuropathy, Poly-Dermatomyositis, Polyarteritis Nodosa, Polyendocrine Deficiency Syndrome, Polyendocrine Deficiency Syndrome Type 1, Polyendocrine Deficiency Syndrome Type 2, Polyglandular Autoimmune Syndrome Type I, Polyglandular Autoimmune Syndrome Type II, Polyglandular Autoimmune Syndrome Type III, Polymyositis, Primary Biliary Cirrhosis, Primary Glomerulonephritis, Primary Sclerosing Cholangitis, Psoriasis, Psoriatic Arthritis, Rasmussen's Encephalitis, Raynaud's Disease, Relapsing Polychondritis, Retrobulbar neuritis, Rheumatic Diseases, Rheumatoid Arthritis, Scleroderma, Sensory neuropathies (paraneoplastic), Sjogren's Syndrome, Stiff-Person Syndrome, Subacute Thyroiditis, Subacute autonomic neuropathy, Sydenham Chorea, Sympathetic Ophthalmitis, Systemic Lupus Erythematosus, Transverse myelitis, Type 1 Diabetes, Ulcerative Colitis, Vasculitis, Vitiligo, Wegener's Granulomatosis, acrocyanosis, anaphylacetic reaction, autoimmune inner ear disease, bilateral sensorineural hearing loss, cold agglutinin hemolytic anemia, cold-induced immune hemolytic anemia, idiopathic endolymphatic hydrops, idiopathic progressive bilateral sensorineural hearing loss, immune-mediated inner ear disease, and mixed autoimmune hemolysis.

In some embodiments, the autoimmune disease is characterized by a proliferation of T-cells, e.g., type 1 diabetes, lupus, and multiple sclerosis, and other pathological states such as graft rejection induced by the presentation of a foreign antigen such as a graft in response to a disease condition (e.g., kidney failure). Other examples of diseases characterized by proliferation of cells include cirrhosis of the liver and restenosis.

In some embodiments, the autoimmune disease to be treated is an autoimmune disease other than type 1 diabetes, lupus, multiple sclerosis, graft rejection induced by the presentation of a foreign antigen., cirrhosis of the liver, or restenosis.

In some embodiments, the peptide is administered before, during, and/or after a transplant to delay the onset of graft-versus-host disease (GVHD). The transplant may be, for example, an xenograft or an allograft, such as an allogeneic stem cell, bone marrow, or organ transplant.

In some embodiments, the method further comprises administering at least one additional therapeutic agent selected from an interleukin-1 (IL-1) inhibitor, IL-1ra, anakinra, a TNF-alpha inhibitor, a soluble TNF-alpha receptor, etanercept, an anti-TNF-alpha antibody, infliximab, a D2E7 antibody, methotrexate, a soluble form of CTLA4, and a modulator of glucocorticoid receptor.

In some embodiments, the method includes administration of one or more agents selected from among a corticosteroid (such as prednisone), nonsteroid drug such as azathioprine, cyclophosphamide, methotrexate, mycophenolate, mofetil, sirolimus, rituximab, tacrolimus, cyclosporine, or other immunosuppressive agent.

Symptoms of autoimmune disorders can vary widely depending on the type of disease. Commonly observed symptoms include fatigue, dizziness, malaise, and fever. Other symptoms that may be observed in one or more autoimmune disorders include chills, weight loss, skin rashes, vasculitis, polyarthralgia, patchy hair loss, oral and nasal sores, lymph-node enlargement, gastric problems, generalized pain, which may be located in the joints in the case of arthritis, enlarged glands, such as the thyroid in the case of Grave's disease, heart palpitations, dermal blisters and lesions, muscle weakness.

The HYD1 peptides can be formulated according to known methods for preparing pharmaceutically useful compositions. Formulations are described in a number of sources which are well known and readily available to those skilled in the art. For example, Remington's Pharmaceutical Science (Martin, E. W., 1995, Easton Pa., Mack Publishing Company, 19^{th} ed.) describes formulations which can be used in connection with the subject invention. Formulations suitable for administration include, for example, aqueous sterile injection solutions, which may contain antioxidants, buffers, bacteriostats, and solutes that render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the compositions of the subject invention can include other agents conventional in the art having regard to the type of formulation in question.

The HYD1 peptides can be formulated as a pharmaceutically acceptable salt. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts.

Pharmaceutically acceptable salts of compounds may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

As used herein, the term “analogs” refers to compounds which are substantially the same as another compound but which may have been modified by, for example, adding side groups, oxidation or reduction of the parent structure. Analogs of the HYD1 peptides, and other agents disclosed herein, can be readily prepared using commonly known standard reactions. These standard reactions include, but are not limited to, hydrogenation, alkylation, acetylation, and acidification reactions. Chemical modifications can be accomplished by those skilled in the art by protecting all functional groups present in the molecule and deprotecting them after carrying out the desired reactions using standard procedures known in the scientific literature (Greene, T. W. and Wuts, P. G. M. “Protective Groups in Organic Synthesis” John Wiley & Sons, Inc. New York. 3rd Ed. pg. 819, 1999; Honda, T. et al. Bioorg. Med. Chem. Lett., 1997, 7:1623-1628; Honda, T. et al. Bioorg. Med. Chem. Lett., 1998, 8:2711-2714; Konoike, T. et al. J. Org. Chem., 1997, 62:960-966; Honda, T. et al. J. Med. Chem., 2000, 43:4233-4246; each of which are hereby incorporated herein by reference in their entirety). Analogs, fragments, and variants of the HYD1 peptides exhibiting the desired biological activity (such as reducing in T-cell numbers and/or promoting bone retention) can be identified or confirmed using cellular assays or other in vitro or in vivo assays.

Therapeutic application of the HYD1 peptides (cyclic peptides and non-cyclic peptides) and compositions comprising them can be accomplished by any suitable therapeutic method and technique presently or prospectively known to those skilled in the art. Further, the HYD1 peptides can be used as starting materials or intermediates for the preparation of other useful compounds and compositions.

The HYD1 peptides may be locally administered at one or more anatomical sites, optionally in combination with a pharmaceutically acceptable carrier such as an inert diluent. HYD1 peptides may be systemically administered, such as intravenously or orally, optionally in combination with a pharmaceutically acceptable carrier such as an inert diluent, or an assimilable edible carrier for oral delivery. They may be enclosed in hard or soft shell gelatin capsules, may be compressed into tablets, or may be incorporated directly with the food of the patient's diet. For oral therapeutic administration, the peptides may be combined with one or more excipients and used in the form of ingestible tablets, buccal tablets, troches, capsules, elixirs, suspensions, syrups, wafers, aerosol sprays, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the HYD1 peptides may be incorporated into sustained-release preparations and devices.

The active agent (e.g., cyclized or non-cyclized HYD1 peptides) may also be administered intravenously or intraperitoneally by infusion or injection. As shown in Table 2, the cyclic peptide MTI-101 demonstrates favorable stability in plasma, indicating that it is not rapidly degraded by proteases.

TABLE 2
Stability

| Species | Stability - 24 Hour | Stability - 24 Hour |
|---|---|---|
| (Plasma) | Room Temperature | 4 Degrees Celsius |
| *Rat | 90% | 94% |
| *Dog | 67% | 83% |
| Mouse | 85% | Not Determined |

Solutions of the active agent can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the HYD1 peptides which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating the HYD1 peptides in the required amount in an appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the HYD1 peptides may be applied in pure-form, i.e., when they are liquids. However, it will generally be desirable to administer them topically to the skin as compositions, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the peptide can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver the peptides to the skin are disclosed in Jacquet et al. (U.S. Pat. No. 4,608,392), Geria (U.S. Pat. No. 4,992,478), Smith et al. (U.S. Pat. No. 4,559,157) and Woltzman (U.S. Pat. No. 4,820,508).

Useful dosages of the pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Accordingly, the present invention includes a pharmaceutical composition comprising HYD1 peptide, optionally in combination with a biologically active agent, and a pharmaceutically acceptable carrier. Pharmaceutical compositions adapted for oral, topical or parenteral administration, comprising an amount of HYD1 peptide of the invention constitute a preferred embodiment of the invention. The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition. Advantageously, in some embodiments, administration of the HYD1 peptides does not induce weight loss or overt signs of toxicity in the subject.

Depending upon the disorder or disease condition to be treated, a suitable dose(s) may be that amount that will reduce the number of one or more populations of T-cells; and/or increase the number of osteoblasts and/or reduce the number of osteoclasts. In the context of bone deficiency, a suitable dose(s) can be that which will ameliorate, eliminate, or delay the onset of one or more symptoms of the bone deficiency. In the context of an autoimmune disorder, a suitable dose(s) can be that which will ameliorate, eliminate, or delay the onset of one or more symptoms of the autoimmune disorder.

Administration of a HYD1 peptide can be continuous or at distinct intervals, as can be determined by a person of ordinary skill in the art.

To provide for the administration of such dosages for the desired therapeutic treatment, in some embodiments, pharmaceutical compositions of the invention can comprise between about 0.1% and 45%, and especially, 1 and 15%, by weight of the total of one or more of the peptides of the invention based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Mammalian species which benefit from the disclosed methods and compositions include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Other species that may benefit from the disclosed methods include fish, amphibians, avians, and reptiles. As used herein, the terms “patient”, “subject”, and “individual” are used interchangeably and are intended to include such human and non-human species (of any gender, e.g., male or female). Likewise, the in vivo methods can be carried out in vitro and the in vitro methods of the present invention can be carried out on cells of such human and non-human species.

The compositions and methods of the invention may use fusion proteins comprising HYD1 peptides. Fusion proteins comprise one or more heterologous peptide sequences (e.g., tags that facilitate purification of the peptides of the invention (see, for example, U.S. Pat. No. 6,342,362, hereby incorporated by reference in its entirety; Altendorf et al. [1999-WWW, 2000] “Structure and Function of the Fₒ Complex of the ATP Synthase from Escherichia Coli,” J. of Experimental Biology 203:19-28, The Co. of Biologists, Ltd., G. B.; Baneyx [1999] “Recombinant Protein Expression in Escherichia coli,” Biotechnology 10:411-21, Elsevier Science Ltd.; Eihauer et al. [2001] “The FLAG™ Peptide, a Versatile Fusion Tag for the Purification of Recombinant Proteins,” J. Biochem Biophys Methods 49:455-65; Jones et al. [1995] J. Chromatography 707:3-22; Jones et al. [1995] “Current Trends in Molecular Recognition and Bioseparation,” J. of Chromatography A. 707:3-22, Elsevier Science B. V.; Margolin[2000] “Green Fluorescent Protein as a Reporter for Macromolecular Localization in Bacterial Cells,” Methods 20:62-72, Academic Press; Puig et al. [2001] “The Tandem Affinity Purification (TAP) Method: A General Procedure of Protein Complex Purification,” Methods 24:218-29, Academic Press; Sassenfeld [1990] “Engineering Proteins for Purification,” TibTech 8:88-93; Sheibani [1999] “Prokaryotic Gene Fusion Expression Systems and Their Use in Structural and Functional Studies of Proteins,” Prep. Biochem. & Biotechnol. 29(1):77-90, Marcel Dekker, Inc.; Skerra et al. “Applications of a Peptide Ligand for Streptavidin: the Strep-tag”, Biomolecular Engineering 16:79-86, Elsevier Science, B. V.; Smith [1998] “Cookbook for Eukaryotic Protein Expression: Yeast, Insect, and Plant Expression Systems,” The Scientist 12(22):20; Smyth et al. [2000] “Eukaryotic Expression and Purification of Recombinant Extracellular Matrix Proteins Carrying the Strep II Tag”, Methods in Molecular Biology, 139:49-57; Unger “Show Me the Money: Prokaryotic Expression Vectors and Purification Systems,” The Scientist 11(17):20, each of which is hereby incorporated by reference in their entireties), or commercially available tags from vendors such as such as STRATAGENE (La Jolla, Calif.), NOVAGEN (Madison, Wis.), QIAGEN, Inc., (Valencia, Calif.), or InVitrogen (San Diego, Calif.).

In other embodiments, peptides of the subject invention can be fused to heterologous polypeptide sequences that have adjuvant activity (a polypeptide adjuvant). Non-limiting examples of such polypeptides include heat shock proteins (hsp) (see, for example, U.S. Pat. No. 6,524,825, the disclosure of which is hereby incorporated by reference in its entirety).

Peptides as described herein may be synthesized by methods well known in the art, including recombinant DNA methods and chemical synthesis. Chemical synthesis may generally be performed using standard solution phase or solid phase peptide synthesis techniques, in which a peptide linkage occurs through the direct condensation of the amino group of one amino acid with the carboxy group of the other amino acid with the elimination of a water molecule. Peptide bond synthesis by direct condensation, as formulated above, requires suppression of the reactive character of the amino group of the first and of the carboxyl group of the second amino acid. The masking substituents must permit their ready removal, without inducing breakdown of the labile peptide molecule.

In solution phase synthesis, a wide variety of coupling methods and protecting groups may be used (see Gross and Meienhofer, eds., “The Peptides: Analysis, Synthesis, Biology,” Vol. 1-4 (Academic Press, 1979); Bodansky and Bodansky, “The Practice of Peptide Synthesis,” 2d ed. (Springer Verlag, 1994)). In addition, intermediate purification and linear scale up are possible. Those of ordinary skill in the art will appreciate that solution synthesis requires consideration of main chain and side chain protecting groups and activation method. In addition, careful segment selection is necessary to minimize racemization during segment condensation. Solubility considerations are also a factor.

Solid phase peptide synthesis uses an insoluble polymer for support during organic synthesis. The polymer-supported peptide chain permits the use of simple washing and filtration steps instead of laborious purifications at intermediate steps. Solid-phase peptide synthesis may generally be performed according to the method of Merrifield et al., J. Am. Chem. Soc., 1963, 85:2149, which involves assembling a linear peptide chain on a resin support using protected amino acids. Solid phase peptide synthesis typically utilizes either the Boc or Fmoc strategy, which are well known in the art.

Those of ordinary skill in the art will recognize that, in solid phase synthesis, deprotection and coupling reactions must go to completion and the side-chain blocking groups must be stable throughout the synthesis. In addition, solid phase synthesis is generally most suitable when peptides are to be made on a small scale.

Acetylation of the N-terminal can be accomplished by reacting the final peptide with acetic anhydride before cleavage from the resin. C-amidation is accomplished using an appropriate resin such as methylbenzhydrylamine resin using the Boc technology.

The peptides disclosed herein may be modified by attachment of a second molecule that confers a desired property upon the peptide, such as increased half-life in the body, for example, pegylation. Such modifications also fall within the scope of the term “variant” as used herein.

Covalent attachment of a molecule or solid support may generally be achieved by first reacting the support material with a bifunctional reagent that will also react with a functional group, such as a hydroxyl, thiol, carboxyl, ketone or amino group, on the modulating agent. A preferred method of generating a linkage is via amino groups using glutaraldehyde. A peptide may be linked to cellulose via ester linkages. Similarly, amide linkages may be suitable for linkage to other molecules such as keyhole limpet hemocyanin or other support materials.

Although HYD1 peptides as described herein may preferentially bind to specific tissues or cells, and thus may be sufficient to target a desired site in vivo, it may be beneficial for certain applications to include an additional targeting agent. Accordingly, a targeting agent may also, or alternatively, be linked to a HYD1 peptide to facilitate targeting to one or more specific tissues. As used herein, a “targeting agent,” may be any substance (such as a compound or cell) that, when linked to a HYD1 peptide, enhances the transport of the HYD1 peptide to a target tissue, thereby increasing the local concentration of the HYD1 peptide. Targeting agents include antibodies or fragments thereof, receptors, ligands and other molecules that bind to cells of, or in the vicinity of, the target tissue. Known targeting agents include serum hormones, antibodies against cell surface antigens, lectins, adhesion molecules, tumor cell surface binding ligands, steroids, cholesterol, lymphokines, fibrinolytic enzymes and those drugs and proteins that bind to a desired target site.

### Following are Exemplified Embodiments:

### Embodiment 1

A method for reducing the number of activated T-cells in a subject, comprising administering an effective amount of a HYD1 peptide to the subject.

### Embodiment 2

The method of embodiment 1, wherein the subject has, or is at risk of developing, an autoimmune disorder.

### Embodiment 3

A method for promoting bone preservation in a subject, comprising administering an effective amount of a HYD1 peptide to the subject.

### Embodiment 4

The method of embodiment 3, wherein the subject has, or is at risk of developing, a bone deficiency.

### Embodiment 5

A method for treating a bone deficiency and/or an autoimmune disorder in a subject, comprising administering an effective amount of a HYD1 peptide to the subject.

### Embodiment 6

The method of any one of embodiments 1-5, wherein the HYD1 peptide is a cyclic peptide having a chemical structure shown in FIGS. 7-34 or 38-41, or a pharmaceutically acceptable salt thereof

### Embodiment 7

The method of embodiment 6, wherein the cyclic peptide is MTI-101 (shown in FIGS. 7 and 23), or a pharmaceutically acceptable salt thereof.

### Embodiment 8

The method of embodiment 6, wherein the cyclic peptide has a chemical structure shown in FIGS. 39-41, or a pharmaceutically acceptable salt thereof, wherein R¹ through R⁵ and R⁶ through R¹⁰ are substituents of natural or unnatural amino acids, wherein a sequence of amino acids with R¹ through R⁵ is a non-recognition sequence and a sequence of amino acids with R⁶ through R¹⁰ is a recognition sequence.

### Embodiment 9

The method of embodiment 8, wherein the non-recognition sequence is KLKLK, KLQLK, QLKLK, KQKLK, or KXKXK where X=sarcosine where either end of the non-recognition sequence is N-terminus, and wherein the recognition sequence is MVVSW, MVVSA, MVVAW, MVASW, MAVSW, AVVSW, N*VVSW, N*VVYW, N*VVAW, AVVAW, N*AVAW, N*VAAW, N*VVAW, N*VLAW, N*VIAW, N*VFAW, or WSVVW, where N*=norleucine, where either end of the recognition sequence is N-terminus.

### Embodiment 10

The method of embodiment 8, wherein the non-recognition sequence is KLKLK, and wherein the recognition sequence is WAVAW, WAVAA, WAVAM, WAVAN*, WAVVN*, WAVSN*, WAVAW, WAAAA, WAAAM, WAAAN*, WAAVW, WAAVA, WAAVM, WAAVN*, WAASN*, WVVAW, WVVAA, WVVAM, WVVAN*, WVVVW, WVVVA, WVVVM, WVVVN*, WVVSN*, WVAVN*, WVAVW, WVAVA, WVAVM, WVAVN*, WVASN*, WSVVW, WSVAA, WSVAM, WSVAN*, WSVVW, WSVVA, WSVVM, WSVVN*, WSVSW, WSVSA, WSVSM, WSVSN*, WSAAW, WSAAA, WSAAM, WSAAN*, WSAVW, WSAVA, WSAVM, WSAVN*, WSASW, WSASA, WSASM, WSASN*, WYVAW, WYVAA, WYVAM, WYVAN*, WYVVW, WYVVA, WYVVM, WYVVN*, WYVSW, WYVSA, WYVSM, WYVSN*, WYAAW, WYAAA, WYAAM, WYAAN*, WYAVW, WYAVA, WYAVM, WYAVN*, WYASW, WYASA, WYASM, WYASN*, AAVAA, AAVAM, AAVAN*, AAVVN*, AAVSN*, AAAAA, AAAAM, AAAAN*, AAAVW, AAAVA, AAAVM, AAAVN*, AAASM, AAASN*, AVVAW, AVVAA, AVVAM, AVVAN*, AVVVA, AVVVM, AVVVN*, AVVSN*, AVAAW, AVAAM, AVAAN*, AVAVA, AVAVM, AVAVN*, AVASN*, ASVAW, ASVAA, ASVAM, ASVAN*, ASVVW, ASVVA, ASVVM, ASVVN*, ASVSA, ASVSM, ASVSN*, ASAAW, ASAAA, ASAAM, ASAAN*, ASAVW, ASAVA, ASAVM, ASAVN*, ASASA, ASASM, ASASN*, AYVAW, AYVAA, AYVAM, AYVAN*, AYVVW, AYVVA, AYVVM, AYVVN*, AYVSW, AYVSA, AYVSM, AYVSN*, AYAAW, AYAAA, AYAAM, AYAAN*, AYAVW, AYAVA, AYAVM, AYAVN*, AYASW, AYASA, AYASM, AYASN*, MAVAA, MAVAM, MAVAN*, MAVVN*, MAVSN*, MAAAA, MAAAM, MAAAN*, MAAVW, MAAVA, MAAVM, MAAVN*, MAASN*, MVVAW, MVVAA, MVVAM, MVVAN*, MVVVM, MVVVN*, MVVSN*, MVVAM, MVAAN*, MVVVM, MVAVN*, MVASN*, MSVAW, MSVAA, MSVAM, MSVAN*, MSVVW, MSVVA, MSVVM, MSVVN*, MSVSM, MSVSN*, MSAAW, MSAAA, MSAAM, MSAAN*, MSAVW, MSAVA, MSAVM, MSAVN*, MSASM, MSASN*, MYVAW, MYVAA, MYVAM, MYVAN*, MYVVW, MYVVA, MYVVM, MYVVN*, MYVSW, MYVSA, MYVSM, MYVSN*, MYAAW, MYAAA, MYAAM, MYAAN*, MYAVW, MYAVA, MYAVM, MYAVN*, MYASW, MYASA, MYASM, or MYASN*, where N*=norleucine, where either end of the recognition sequence is N-terminus.

### Embodiment 11

The method of any one of embodiments 1-5, wherein the HYD1 peptide is a non-cyclic peptide (e.g., linear peptide) selected from among: KIKMVISWKG (HYD1; SEQ ID NO:1); AIKMVISWKG (SEQ ID NO:2; HYD8); AIKMVISWKG (SEQ ID NO:3; HYDE); AIKMVISWKG (SEQ ID NO:4; HYD2); AKMVISW (SEQ ID NO:5); AKMVISWKG (SEQ ID NO:6); IAMVISW (SEQ ID NO:7); IAMVISWKG (SEQ ID NO:8); IKAVISW (SEQ ID NO:9); IKAVISWKG (SEQ ID NO:10); IKMAISW (SEQ ID NO:11); IKMAISWKG (SEQ ID NO:12); IKMVASW (SEQ ID NO:13); IKMVASWKG (SEQ ID NO:14); IKMVIAW (SEQ ID NO:15); IKMVIAWKG (SEQ ID NO:16); IKMVISA (SEQ ID NO:17); IKMVISAKG (SEQ ID NO:18); IKMVISW (SEQ ID NO:19); IKMVISWAG (SEQ ID NO:20); KMVISWKA (SEQ ID NO:21); IKMVISWKG (SEQ ID NO:22; HYD18; (-K)HYD1); ISWKG (SEQ ID NO:23); KAKMVISWKG (SEQ ID NO:24); KIAMVISWKG (SEQ ID NO:25; HYD7); KIAMVISWKG (SEQ ID NO:26); KIKAVISWKG (SEQ ID NO:27); KIKMAISWKG (SEQ ID NO:28); KIKMV (SEQ ID NO:29); KIKMVASWKG (SEQ ID NO:30); KIKMVI (SEQ ID NO:31; HYD16); KIKMVIAWKG (SEQ ID NO:32); KIKMVIS (SEQ ID NO:33; HYD15); KIKMVISAKG (SEQ ID NO:34); KIKMVISW (SEQ ID NO:35; HYD14); KIKMVISWAG (SEQ ID NO:36); KIKMVISWK (SEQ ID NO:37; HYD17; HYD1(-G)); KIKMVISWKA (SEQ ID NO:38); KMVISWKG (SEQ ID NO:39; HYD9); LSWKG (SEQ ID NO:40; HYD12); MVISWKG (SEQ ID NO:41; HYD10); SWKG (SEQ ID NO:42; HYD13); VISWKG (SEQ ID NO:43; HYD11); WIKSMKIVKG (SEQ ID NO:44); KMVIXW (SEQ ID NO:46); IKMVISWXX (SEQ ID NO:48); and KMVISWXX (SEQ ID NO:49); wherein X is any amino acid (traditional or non-traditional amino acid).

### Embodiment 12

The method of embodiment 4 or 5, wherein the bone deficiency is caused by an osteopenic disorder.

### Embodiment 13

The method of embodiment 12, wherein the osteopenic disorder is selected from among osteoporosis, Paget's disease, lytic bone metastases, periodontitis, rheumatoid arthritis, and bone loss due to immobilization.

### Embodiment 14

The method of embodiment 4 or 5, wherein the subject has a cancer that increases osteoclast activity and/or induces bone resorption.

### Embodiment 15

The method of any preceding embodiment, wherein the subject does not have cancer or other proliferation disorders.

### Embodiment 16

The method of embodiment 5, wherein the subject has an autoimmune disorder.

### Embodiment 17

The method of embodiment 2, 5, or 16, wherein the autoimmune disorder is selected from among: AIDS-associated myopathy, AIDS-associated neuropathy, Acute disseminated encephalomyelitis, Addison's Disease, Alopecia Areata, Anaphylaxis Reactions, Ankylosing Spondylitis, Antibody-related Neuropathies, Antiphospholipid Syndrome, Arthritis (e.g., rheumatoid arthritis, psoriatic arthritis, juvenile idiopathic arthritis), Autism, Autoimmune Atherosclerosis, Autoimmune Diabetes Insipidus, Autoimmune Endometriosis, Autoimmune Eye Diseases, Autoimmune Gastritis, Autoimmune Hemolytic Anemia, Autoimmune Hemophilia, Auto immune Hepatitis, Auto immune Interstitial Cystitis, Auto immune Lym pho proliferative Syndrome, Autoimmune Myelopathy, Autoimmune Myocarditis, Autoimmune Neuropathies, Autoimmune Oophoritis, Autoimmune Orchitis, Autoimmune Thrombocytopenia, Autoimmune Thyroid Diseases, Autoimmune Urticaria, Autoimmune Uveitis, Autoimmune Vasculitis, Behcet's Disease, Bell's Palsy, Bullous Pemphigoid, CREST, Celiac Disease, Cerebellar degeneration (paraneoplastic), Chronic Fatigue Syndrome, Chronic Rhinosinusitis, Chronic inflammatory demyelinating polyneuropathy, Churg Strauss Syndrome, Connective Tissue Diseases, Crohn's Disease, Cutaneous Lupus, Dermatitis Herpetiformis, Dermatomyositis, Diabetes Mellitus, Discoid Lupus Erythematosus, Drug-induced Lupus, Endocrine Orbitopathy, Glomerulonephritis, Goodpasture Syndrome, Goodpasture's Syndrome, Graft-versus-Host Disease (GVHD), Graves Disease, Guillian-Barre Syndrome, Miller Fisher variant of the Guillian Barre Syndrome, axonal Guillian Barre Syndrome, demyelinating Guillian Bane Syndrome, Hashimoto Thyroiditis, Herpes Gestationis, Human T-cell lymphomavirus-associated myelopathy, Huntington's Disease, IgA Nephropathy, Immune Thrombocytopenic Purpura, Inclusion body myositis, Interstitial Cystitis, Isaacs syndrome, Lambert Eaton myasthenic syndrome, Limbic encephalitis, Lower motor neuron disease, Lyme Disease, MCTD, Microscopic Polyangiitis, Miller Fisher Syndrome, Mixed Connective Tissue Disease, Mononeuritis multiplex (vasculitis), Multiple Sclerosis (relapsing-remitting MS (RRMS), secondary-progressive MS (SPMS), primary-progressive MS (PPMS), and progressive-relapsing MS (PRMS)), Myasthenia Gravis, Myxedema, Meniere Disease, Neonatal LE, Neuropathies with dysproteinemias, Opsoclonus-myoclonus, PBC, POEMS syndrome, Paraneoplastic Autoimmune Syndromes, Pemphigus, Pemphigus Foliaceus, Pemphigus Vulgaris, Pernicious Anemia, Peyronie's Disease, Plaque Psoriasis, Plasmacytoma/myeloma neuropathy, Poly-Dermatomyositis, Polyarteritis Nodosa, Polyendocrine Deficiency Syndrome, Polyendocrine Deficiency Syndrome Type 1, Polyendocrine Deficiency Syndrome Type 2, Polyglandular Autoimmune Syndrome Type I, Polyglandular Autoimmune Syndrome Type II, Polyglandular Autoimmune Syndrome Type III, Polymyositis, Primary Biliary Cirrhosis, Primary Glomerulonephritis, Primary Sclerosing Cholangitis, Psoriasis, Psoriatic Arthritis, Rasmussen's Encephalitis, Raynaud's Disease, Relapsing Polychondritis, Retrobulbar neuritis, Rheumatic Diseases, Rheumatoid Arthritis, Scleroderma, Sensory neuropathies (paraneoplastic), Sjogren's Syndrome, Stiff-Person Syndrome, Subacute Thyroiditis, Subacute autonomic neuropathy, Sydenham Chorea, Sympathetic Ophthalmitis, Systemic Lupus Erythematosus, Transverse myelitis, Type 1 Diabetes, Ulcerative Colitis, Vasculitis, Vitiligo, Wegener's Granulomatosis, acrocyanosis, anaphylacetic reaction, autoimmune inner ear disease, bilateral sensorineural hearing loss, cold agglutinin hemolytic anemia, cold-induced immune hemolytic anemia, idiopathic endolymphatic hydrops, idiopathic progressive bilateral sensorineural hearing loss, immune-mediated inner ear disease, and mixed autoimmune hemolysis.

### Embodiment 18

The method of embodiment 2 or 5, wherein the subject has graft-versus-host disease (GVHD), and the HYD1 peptide is administered to treat the GVHD in the subject.

### Embodiment 19

The method of embodiment 2 or 5, wherein the HYD1 peptide is administered before, during, and/or after a transplant to delay the onset of graft-versus-host disease (GVHD).

### Embodiment 20

The method of embodiment 18 or 19, wherein the transplant comprises an allograft or xenograft.

### Embodiment 21

The method of embodiment 18 or 19, wherein the transplant comprises an allogeneic stem cell, bone marrow, or organ transplant.

### Embodiment 22

The method of any preceding embodiment, wherein at least two different HYD1 peptides are administered to the subject.

### Embodiment 23

The method of any preceding embodiment, further comprising administering a biologically active agent to the subject.

### Embodiment 24

The method of embodiment 23, wherein the biologically agent is:
(a) an agent for treatment of a bone deficiency, for example, an agent selected from among bisphosphonate (e.g., alendronate, risedronate, ibandronate, zoledronic acid), teriparatide, denosumab, and calcitonin; or
(b) an agent for treatment of an autoimmune disorder, for example, an agent selected from among a corticosteroid (such as prednisone), nonsteroid drug such as azathioprine, cyclophosphamide, methotrexate, mycophenolate, mofetil, sirolimus, rituximab, tacrolimus, cyclosporine, or other immunosuppressive agent.

### Embodiment 25

A composition comprising a HYD1 peptide and one or more agents selected from an agent for treatment of a bone deficiency and/or an autoimmune disorder.

### Embodiment 26

The composition of embodiment 25, wherein the HYD1 peptide is a cyclic peptide having a chemical structure shown in FIGS. 7-34 or 38-41, or a pharmaceutically acceptable thereof.

### Embodiment 27

The composition of embodiment 25, wherein the cyclic peptide is MTI-101 (shown in FIGS. 7 and 23), or a pharmaceutically acceptable salt thereof.

### Embodiment 28

The composition of embodiment 26, wherein the cyclic peptide has a chemical structure shown in FIGS. 39-41, or a pharmaceutically acceptable salt thereof, wherein R¹ through R⁵ and R⁶ through R¹⁰ are substituents of natural or unnatural amino acids, wherein a sequence of amino acids with R¹ through R⁵ is a non-recognition sequence and a sequence of amino acids with R⁶ through R¹⁰ is a recognition sequence.

### Embodiment 29

The composition of embodiment 28, wherein the non-recognition sequence is KLKLK, KLQLK, QLKLK, KQKLK, or KXKXK where X=sarcosine where either end of the non-recognition sequence is N-terminus, and wherein the recognition sequence is MVVSW, MVVSA, MVVAW, MVASW, MAVSW, AVVSW, N*VVSW, N*VVYW, N*VVAW, AVVAW, N*AVAW, N*VAAW, N*VVAW, N*VLAW, N*VIAW, N*VFAW, or WSVVW, where N*=norleucine, where either end of the recognition sequence is N-terminus.

### Embodiment 30

The composition of embodiment 28, wherein the non-recognition sequence is KLKLK, and wherein the recognition sequence is WAVAW, WAVAA, WAVAM, WAVAN*, WAVVN*, WAVSN*, WAVAW, WAAAA, WAAAM, WAAAN*, WAAVW, WAAVA, WAAVM, WAAVN*, WAASN*, WVVAW, WVVAA, WVVAM, WVVAN*, WVVVW, WVVVA, WVVVM, WVVVN*, WVVSN*, WVAVN*, WVAVW, WVAVA, WVAVM, WVAVN*, WVASN*, WSVVW, WSVAA, WSVAM, WSVAN*, WSVVW, WSVVA, WSVVM, WSVVN*, WSVSW, WSVSA, WSVSM, WSVSN*, WSAAW, WSAAA, WSAAM, WSAAN*, WSAVW, WSAVA, WSAVM, WSAVN*, WSASW, WSASA, WSASM, WSASN*, WYVAW, WYVAA, WYVAM, WYVAN*, WYVVW, WYVVA, WYVVM, WYVVN*, WYVSW, WYVSA, WYVSM, WYVSN*, WYAAW, WYAAA, WYAAM, WYAAN*, WYAVW, WYAVA, WYAVM, WYAVN*, WYASW, WYASA, WYASM, WYASN*, AAVAA, AAVAM, AAVAN*, AAVVN*, AAVSN*, AAAAA, AAAAM, AAAAN*, AAAVW, AAAVA, AAAVM, AAAVN*, AAASM, AAASN*, AVVAW, AVVAA, AVVAM, AVVAN*, AVVVA, AVVVM, AVVVN*, AVVSN*, AVAAW, AVAAM, AVAAN*, AVAVA, AVAVM, AVAVN*, AVASN*, ASVAW, ASVAA, ASVAM, ASVAN*, ASVVW, ASVVA, ASVVM, ASVVN*, ASVSA, ASVSM, ASVSN*, ASAAW, ASAAA, ASAAM, ASAAN*, ASAVW, ASAVA, ASAVM, ASAVN*, ASASA, ASASM, ASASN*, AYVAW, AYVAA, AYVAM, AYVAN*, AYVVW, AYVVA, AYVVM, AYVVN*, AYVSW, AYVSA, AYVSM, AYVSN*, AYAAW, AYAAA, AYAAM, AYAAN*, AYAVW, AYAVA, AYAVM, AYAVN*, AYASW, AYASA, AYASM, AYASN*, MAVAA, MAVAM, MAVAN*, MAVVN*, MAVSN*, MAAAA, MAAAM, MAAAN*, MAAVW, MAAVA, MAAVM, MAAVN*, MAASN*, MVVAW, MVVAA, MVVAM, MVVAN*, MVVVM, MVVVN*, MVVSN*, MVAAM, MVAAN*, MVAVM, MVAVN*, MVASN*, MSVAW, MSVAA, MSVAM, MSVAN*, MSVVW, MSVVA, MSVVM, MSVVN*, MSVSM, MSVSN*, MSAAW, MSAAA, MSAAM, MSAAN*, MSAVW, MSAVA, MSAVM, MSAVN*, MSASM, MSASN*, MYVAW, MYVAA, MYVAM, MYVAN*, MYVVW, MYVVA, MYVVM, MYVVN*, MYVSW, MYVSA, MYVSM, MYVSN*, MYAAW, MYAAA, MYAAM, MYAAN*, MYAVW, MYAVA, MYAVM, MYAVN*, MYASW, MYASA, MYASM, or MYASN*, where N*=norleucine, where either end of the recognition sequence is N-terminus.

### Embodiment 31

The composition of embodiment 25, wherein the HYD1 peptide is non-cyclic peptide (e.g., linear peptide) selected from among: KIKMVISWKG (HYD1; SEQ ID NO:1); AIAMVISWAG (SEQ ID NO:2; HYD8); AIKMVISWAG (SEQ ID NO:3; HYDE); AIKMVISWKG (SEQ ID NO:4; HYD2); AKMVISW (SEQ ID NO:5); AKMVISWKG (SEQ ID NO:6); IAMVISW (SEQ ID NO:7); IAMVISWKG (SEQ ID NO:8); IKAVISW (SEQ ID NO:9); IKAVISWKG (SEQ ID NO:10); IKMAISW (SEQ ID NO:11); IKMAISWKG (SEQ ID NO:12); IKMVASW (SEQ ID NO:13); IKMVASWKG (SEQ ID NO:14); IKMVIAW (SEQ ID NO:15); IKMVIAWKG (SEQ ID NO:16); IKMVISA (SEQ ID NO:17); IKMVISAKG (SEQ ID NO:18); IKMVISW (SEQ ID NO:19); IKMVISWAG (SEQ ID NO:20); KMVISWKA (SEQ ID NO:21); IKMVISWKG (SEQ ID NO:22; HYD18; (-K)HYD1); ISWKG (SEQ ID NO:23); KAKMVISWKG (SEQ ID NO:24); KIAMVISWAG (SEQ ID NO:25; HYD7); KIAMVISWKG (SEQ ID NO:26); KIKAVISWKG (SEQ ID NO:27); KIKMAISWKG (SEQ ID NO:28); KIKMV (SEQ ID NO:29); KIKMVASWKG (SEQ ID NO:30); KIKMVI (SEQ ID NO:31; HYD16); KIKMVIAWKG (SEQ ID NO:32); KIKMVIS (SEQ ID NO:33; HYD15); KIKMVISAKG (SEQ ID NO:34); KIKMVISW (SEQ ID NO:35; HYD14); KIKMVISWAG (SEQ ID NO:36); KIKMVISWK (SEQ ID NO:37; HYD17; HYD1(-G)); KIKMVISWKA (SEQ ID NO:38); KMVISWKG (SEQ ID NO:39; HYD9); LSWKG (SEQ ID NO:40; HYD12); MVISWKG (SEQ ID NO:41; HYD10); SWKG (SEQ ID NO:42; HYD13); VISWKG (SEQ ID NO:43; HYD11); WIKSMKIVKG (SEQ ID NO:44); KMVIXW (SEQ ID NO:46); IKMVISWXX (SEQ ID NO:48); and KMVISWXX (SEQ ID NO:49); wherein X is any amino acid (traditional or non-traditional amino acid).

### Embodiment 32

The composition of any preceding embodiment, wherein the one or more agents are selected from among:
(a) an agent for treatment of a bone deficiency selected from among bisphosphonate (e.g., alendronate, risedronate, ibandronate, zoledronic acid), teriparatide, denosumab, and calcitonin; or
(b) an agent for treatment of an autoimmune disorder selected from among a corticosteroid (such as prednisone), nonsteroid drug such as azathioprine, cyclophosphamide, methotrexate, mycophenolate, mofetil, sirolimus, rituximab, tacrolimus, cyclosporine, or other immunosuppressive agent.

### DEFINITIONS

As used herein, the terms “administering” or “administer” are defined as the introduction of a substance into cells in vitro or into the body of an individual in vivo by any route (for example, oral, nasal, ocular, rectal, vaginal and parenteral routes). Peptides may be administered individually or in combination with other agents via any route of administration, including but not limited to subcutaneous (SQ), intramuscular (IM), intravenous (IV), intraperitoneal (IP), intradermal (ID), via the nasal, ocular or oral mucosa (IN), or orally. For example, peptides can be administered by direct injection into or on a target site, or systemically (e.g., into the circulatory system).

In the context of the instant invention, the terms “oligopeptide”, “polypeptide”, “peptide” and “protein” can be used interchangeably; however, it should be understood that the invention does not relate to the peptides in natural form, that is to say that they are not in their natural environment but that the peptide may have been isolated or obtained by purification from natural sources or obtained from host cells prepared by genetic manipulation (e.g., the peptides, or fragments thereof, are recombinantly produced by host cells, or by chemical synthesis). HYD1 peptides according to the instant invention may also contain non-natural amino acids, as will be described below. The terms “oligopeptide”, “polypeptide”, “peptide” and “protein” are also used, in the instant specification, to designate a series of residues of any length, typically L-amino acids, connected one to the other, typically by peptide bonds between the α-amino and carboxyl groups of adjacent amino acids. Linker elements can be joined to the peptides of the subject invention, for example, through peptide bonds or via chemical bonds (e.g., heterobifunctional chemical linker elements) as set forth below. Additionally, the terms “amino acid(s)” and “residue(s)” can be used interchangeably.

As used herein, the terms “treat” and “treatment” and grammatical variations thereof refer to both therapeutic treatment and prophylactic or preventative measures, wherein the object is to prevent or slow down (lessen) an undesired physiological change or disorder, such as the development or spread of cancer or other proliferation disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. For example, treatment with a HYD1 peptide may include reduction of one or more activated T-cell populations (CD4⁺, CD8⁺, regulatory T cells (T_{reg})), increase of osteoblasts, and/or decrease of osteoclasts. “Treatment” can also mean prolonging survival as compared to expected survival if not receiving treatment. Those in need of treatment include those already with the condition or disorder as well as those prone to have the condition or disorder or those in which the condition or disorder is to be prevented or onset of the disorder delayed. Optionally, the patient may be identified (e.g., diagnosed) as one suffering from the disease or condition (e.g., bone deficiency and/or autoimmune disorder) prior to administration of the peptide. Without being bound by theory, one or more of the peptides may be used by administering an effective amount to reduce or eliminate T-cells (CD4⁺, CD8⁺, regulatory T cells (T_{reg})) in vivo, for treatment of an autoimmune disorder, and/or to increase osteoblast production and decrease osteoclast production in vivo, for treatment of a bone deficiency such as osteoporosis, bone lesion, or other bone loss.

As used herein, the term “(therapeutically) effective amount” refers to an amount of the peptide effective to treat a disease or disorder in a subject, such as a human or non-human mammal.

As used herein, the term “anti-cancer agent” refers to a substance or treatment (e.g., radiation therapy) that inhibits the function of cancer cells, inhibits their formation, and/or causes their destruction in vitro or in vivo. Examples include, but are not limited to, cytotoxic agents (e.g., 5-fluorouracil, TAXOL), chemotherapeutic agents, and anti-signaling agents (e.g., the PI3K inhibitor LY). In one embodiment, the anti-cancer agent is administered before, during, after administration of the peptide or encoding polynucleotide. In some embodiments, a biologically active agent is included in the composition or method of the invention and the biologically active agent is an anti-cancer agent. In some embodiments, a biologically active agent is included in the composition or method of the invention and the biologically active agent is not an anti-cancer agent.

As used herein, the term “pharmaceutically acceptable salt or prodrug” is intended to describe any pharmaceutically acceptable form (such as an ester, phosphate ester, salt of an ester or a related group) of HYD1 peptide of the invention or other agent, which, upon administration to a subject, provides the mature or base compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The terms “link” or “join” refers to any method known in the art for functionally connecting peptides, including, without limitation, recombinant fusion, covalent bonding, disulfide bonding, ionic bonding, hydrogen bonding, and electrostatic bonding.

The terms “comprising”, “consisting of” and “consisting essentially of” are defined according to their standard meaning. The terms may be substituted for one another throughout the instant application in order to attach the specific meaning associated with each term.

As used in this specification, the singular forms “a”, “an”, and “the” include plural reference unless the context clearly dictates otherwise. Thus, for example, a reference to “a compound” includes one or more such compound. Reference to “a HYD1 peptide” includes one or more of such HYD1 peptide, and so forth.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. Eds. (1991) IRL Press)), each of which are incorporated herein by reference in their entirety.

All patents, patent applications, provisional applications, and publications referred to or cited herein, supra or infra, are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1

### Effect of a Cyclic Peptide (MTI-101) on a CD4 Population and its Active Subset in the Bone Marrow, Spleen, and Lymph Node Compartment of Mice

12 C57BL/KaLwRij mice (6-8 weeks old) were injected with 1 million 5TGM1 multiple myeloma cells intravenously via the tail vein injection. A cohort of 6 mice of the same age were utilized as sham controls. After 5 days, the 12 mice were randomized and 6 received intra-peritoneal (i.p.) injections of PBS as vehicle control (V.C) and remaining mice were injected i.p. with 25 mg/kg of the cyclic peptide MTI-101. The tumor was allowed to progress and the day when a V.C.-treated mouse showed signs of hind leg paralysis, the control mouse along with a randomly chosen sham and MTI-101-treated mice were subjected to euthanasia.

Processing Cells from Bone Marrow (BM):

The euthanized mice were dissected open and the entire leg was removed from the pelvic bone. The foot below the ankle joint was removed followed by removal of muscle from around the tibia and the femur. The cleaned bone devoid of muscles or connective tissue was placed in RPMI media supplemented with 10% FBS and 1× penicillin/streptomycin antibiotic. The femur was separated from the tibia and the ends of the bones where cut so as to expose the marrow. The femurs were flushed with a 21 g needle whereas the tibias were flushed with a 25 g needle with the use of RPMI growth media. The flushed BM cells were spun down at 1500 rpm for 10 min and the resulting cell pellet was stored on ice for further processing.

Processing Cells from Spleen (SPL) and Lymph Nodes (LN):

The euthanized mice were dissected open and the spleen and the two sets of Inguinal lymph nodes were harvested and placed in RPMI growth media. The SPL or the LN were then placed on a cell strainer above a petri dish and using a syringe plunger the tissue was ground through the cell strainer. The cells collected below in the petri dish was resuspended in RPMI and spun down at 1500 rpm for 10 min and the resulting cell pellet was stored on ice for further processing.

Preparation of Samples for Flow Cytometry:
(a) The cell pellets were first resuspended in RBC lysis buffer for 5 min at RT. After ensuring complete lysis of RBC, the cells were washed twice with PBS. The cells were then passed through a cell strainer to ensure absence of clumping and then resuspended in staining buffer (PBS+1% FBS). The resuspended cells where counted and aliquoted out in flow tubes such that each flow tube contained 1 million cells per ml of staining buffer.
(b) The suspended cells were first incubated for 30 min on ice with 1 μl of blue florescent reactive dye to distinguish dead cells in the suspended cells. The cells were washed twice with PBS and resuspended in 100 μl of staining buffer. The cells were then stained for desired surface markers using flourochrome-conjugated antibodies. The staining was carried out for 1 hour on ice protected from light. The cells were washed twice with PBS and fixed overnight at 4° C. with 37% formaldehyde.
(c) After overnight fixing, the cells were washed twice with PBS followed by incubation in a permeablization reagent to stain for intracellular markers. After 30 min staining process the cells were washed twice and then resuspended in PBS for cell staining analysis using Flow Cytometer LSRII.

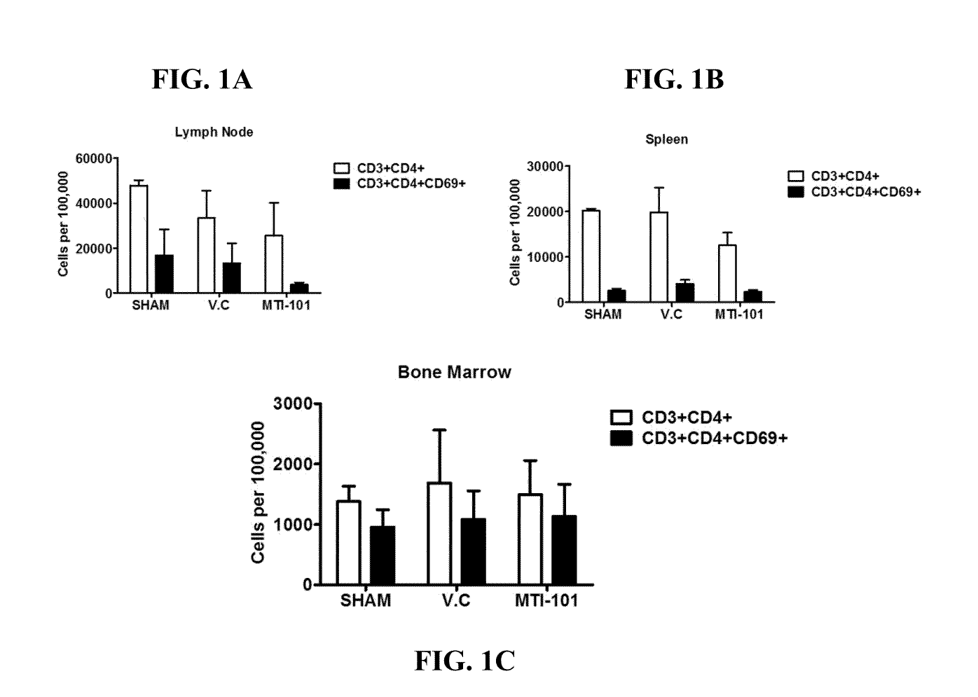

The CD4 cell population, also called T-helper cells, co-operate with other immune cells to mount an immune response against infection or tumor cells. In the present experiment, within the BM compartment, no change was observed in the total CD4 cell population in the sham, VC and MTI-101 treated mice, as shown in FIG. 1C. Additionally, even though a very high percentage of the total CD4 cells were activated CD4 cells (comprising of approximately 70% of the total population), these cell population remained comparable in all the three groups of mice. As shown in FIG. 1B, in the spleen compartment, the total CD4 cell population was decreased by 40% in the MTI-101 treated mice when compared to both sham and V.C. group, both of which had similar numbers of CD4 cells. However in spleen the numbers of activated CD4 cells in V.C. treated mice were approximately 58% higher when compared numbers of activated CD4 cells found in the sham. Interestingly, treatment with MTI-101 decreased the activated CD4 cell to levels comparable to the sham mice. Surprisingly, in the lymph node, the CD4 total cell population fell by 30% in VC treated mice and even more dramatic 50% in the MTI-101 treated mice when compared to the sham mice, as shown in FIG. 1A. This decrease in total CD4 cells equated to a modest 21% decrease in V.C.-treated mice and a very drastic 80% in the MTI-101 treated mice of the activated CD4 cell population. Taken together, these data suggest that MTI-101 spares the CD4 cell population of the BM. However, it has a very detrimental effect on the CD4 population in lymph node and the spleen compartment. In both these organs, MTI-101 significantly decreases the total CD4 cell population. In the spleen, MTI-101 reverses the increase in activated CD4 T cells in the V.C group of mice to levels comparable to sham mice. More importantly, MTI-101 drastically depletes the activated CD4 cell population within the lymph node to levels that are well below those observed in BM and spleen.

### Example 2

### Effect of MTI-101 on a CD8 Population and its Active Subset in the Bone Marrow, Spleen, and Lymph Node of Mice

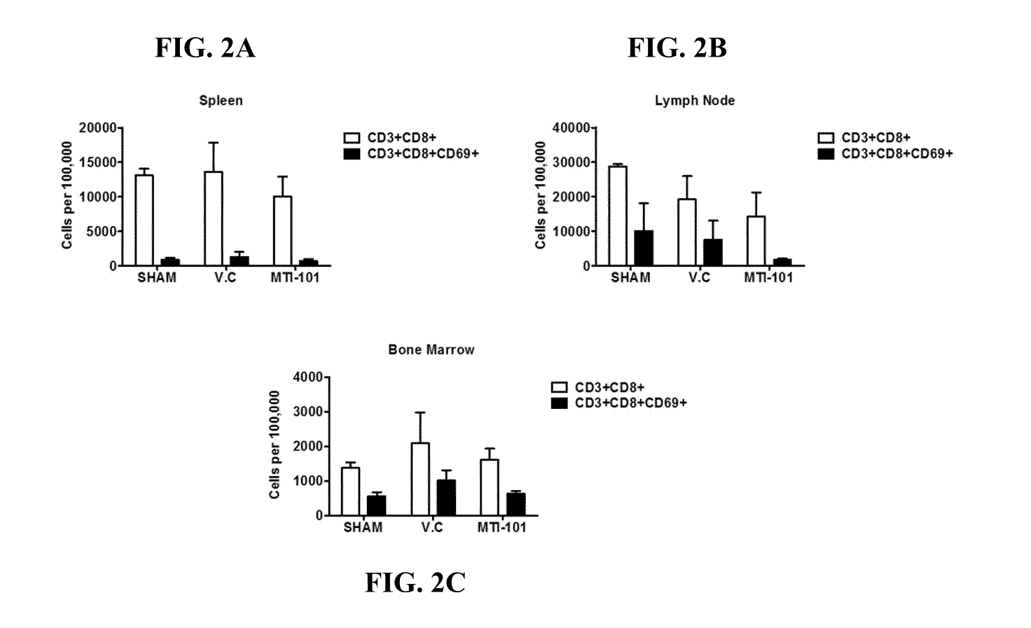

CD8 cell are also called the cytotoxic T cells that can either co-operate with other immune cells or can directly mount an immune response against infection or tumor cells. Unlike the observations made with the CD4 cells, within the BM compartment, the V.C.-treated mice showed a 50% increase in the total CD8 cell population that also resulted in a significant 80% increase in the activated CD8 cells when compared to the sham mice, as shown in FIG. 2C. Interestingly, treatment with MTI-101 restored not only the total CD8 cell population but also induced an decrease in activated CD8 cell population to levels comparable to those seen in sham mice. Similar to observations with the CD4 cells, in the spleen, the total CD8 cell population was decreased by a modest 25% in the MTI-101 treated mice when compared to both sham and V.C group, both of which had similar numbers of CD8 cells, as shown in FIG. 2A. Also, in spleen the numbers of activated CD8 cells were approximately 45% higher in V.C.-treated group when compared to the sham mice. Treatment with MTI-101 reversed this increased and brought the levels of CD8 activated cells to levels that where even lower than the sham mice (by 25%). Finally in the lymph node, the CD8 total cell population fell by 33% in VC treated mice and even more dramatically by 51% in the MTI-101 treated mice when compared to the sham mice, as shown in FIG. 2B. This decrease in total CD8 cells equated to a modest 25% decrease in V.C.-treated mice and a very significant 83% in the MTI-101 treated mice of the activated CD8 cell population. Taken together, these data suggest that MTI-101 has a very detrimental effect on the CD8 population in all the three organs profiled. In the BM, MTI-101 completely reversed the effects of increased CD8 total and activated population seen in the V.C.-treated mice. Additionally, MTI-101 treatment induced a depletion in activated CD8 cells to levels that were even below those observed in sham mice. Finally, in the lymph node, very similar to the profile of CD4 cells, MTI-101 not only decreased the total CD8 cell population but it also induced an even higher reduction in the activated CD8 cell population.

### Example 3

### Effect of MTI-101 on the Regulatory T Cells (Treg) in the Bone Marrow, Spleen, and Lymph Node Compartment of Mice

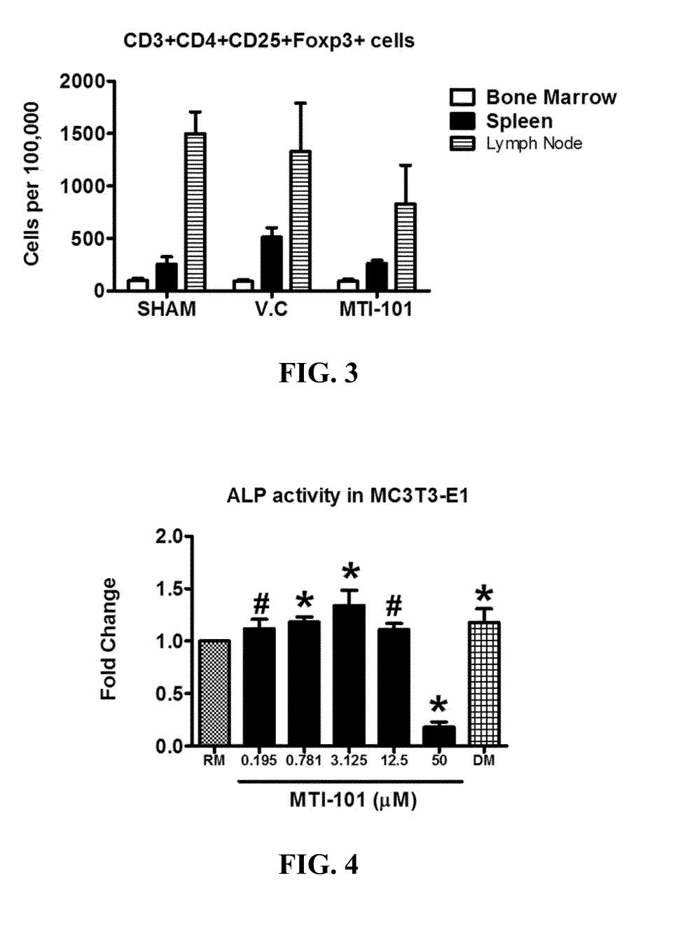

Mouse pre-osteoblastic MC3T3-E1 cells were obtained from ATCC and were seeded at 2×10⁴ cells/well in a 48-well plate. The cells were cultured overnight in DMEM (without ascorbic acid) supplemented with 10% FBS and penicillin/streptomycin. The next day the growth media was changed and the cells were cultured, either in growth media containing the bone differentiation cocktail (10 mM β-glycreophosphate, 100 nM Dexamethasone and 50 μg/ml of Ascorbic acid) or in growth media containing 0.195 to 50 μM of MTI-101. This culture regimen was replaced every alternate day for a total of seven days. On the seventh day the cells were lysed and alkaline phosphatase (ALP) activity was measured at 405 nm using the method as described by the manufacturer (Abcam). Results are shown in FIG. 3.

Regulatory T (Treg) cells play a very important role in modulation of immune response against tumor cells. In the present experiment, within the BM compartment, no change was observed in the total active Treg cell population in the sham, VC or the MTI-101 treated mice, as shown in FIG. 3. Not surprisingly, in the spleen compartment, the total Treg cell population was doubled in the V.C.-treated mice when compared to the sham mice. Treatment of mice with MTI-101 reversed this doubling and brought the levels of Treg cells to levels that were comparable to those seen in sham mice. In the lymph nodes, unlike in the spleen, the active Treg levels were comparable in the sham and V.C.-treated mice. However, mice treated with MTI-101 showed a 45% drop in their active Treg levels compared to the sham mice. Taken together, these data suggests while MTI-101 spares the active T cell population of the BM, it very significantly reverses the high levels of active Treg cells seen in V.C.-treated mice to levels comparable to sham. Also, MTI-101 treatment causes a depletion of active Treg cells in the lymph nodes to levels far below those seen in V.C.-treated and sham mice.

### Example 4

### Effect of MTI-101 on Alkaline Phosphatase in Mouse Pre-Osteoblastic MC3T3-E1 Cells

Murine macrophage RAW 264.7 cells were plated at 8×10³ cells/well in a 24-well plate. After overnight incubation, the media was changed (day 1) and cells were stimulated to differentiate into osteoclast by addition of 50 ng/ml of recombinant murine RANKL. The process was repeated again on day 3 and day 5 post-cell plating. On day six, after confirming the formation of osteoclast by looking for multi-nucleated cells under light microscope, the osteoclasts were treated with 6.25 to 50 μM of MTI-101 for 24 hrs. At the end of the treatment, to evaluate the number of osteoclasts formed, a tartarate-resistant acid phosphatase (TRAP) staining was performed using leukocyte acid phosphatase kit (SIGMA ALDRICH). In parallel, to determine the effect of MTI-101 on RAW 264.7 cell proliferation and cell death, a MTT and Flow cytometry assay was performed respectively. Results are shown in FIG. 4.

ALP is an enzyme marker for osteoblast formation. Osteoblast increases the activity of ALP in order to maintain uniform calcification of the bone. While culturing the cells in RM did not change ALP activity, addition of differentiation cocktail up-regulated the activity of the enzyme as expected. Interestingly, MTI-101 at lower doses of 0.78 and 3.125 μM increased ALP activity to levels comparable to those seen in cells cultured in DM. However, this increase in ALP activity was lost when dose of MTI-101 was escalated to 12.5 μM and at 50 μM MTI-101 significantly suppressed ALP activity when compared to cells cultured in RM. These results demonstrate that MTI-101 has a positive effect on osteoblast formation; however, this effect on osteoblastogenesis is only confined to the lower doses of MTI-101.

### Example 5

### Effect of MTI-101 on RAW 264.7 Cells and on RAW 264.7 Cell-Differentiated Osteoclasts

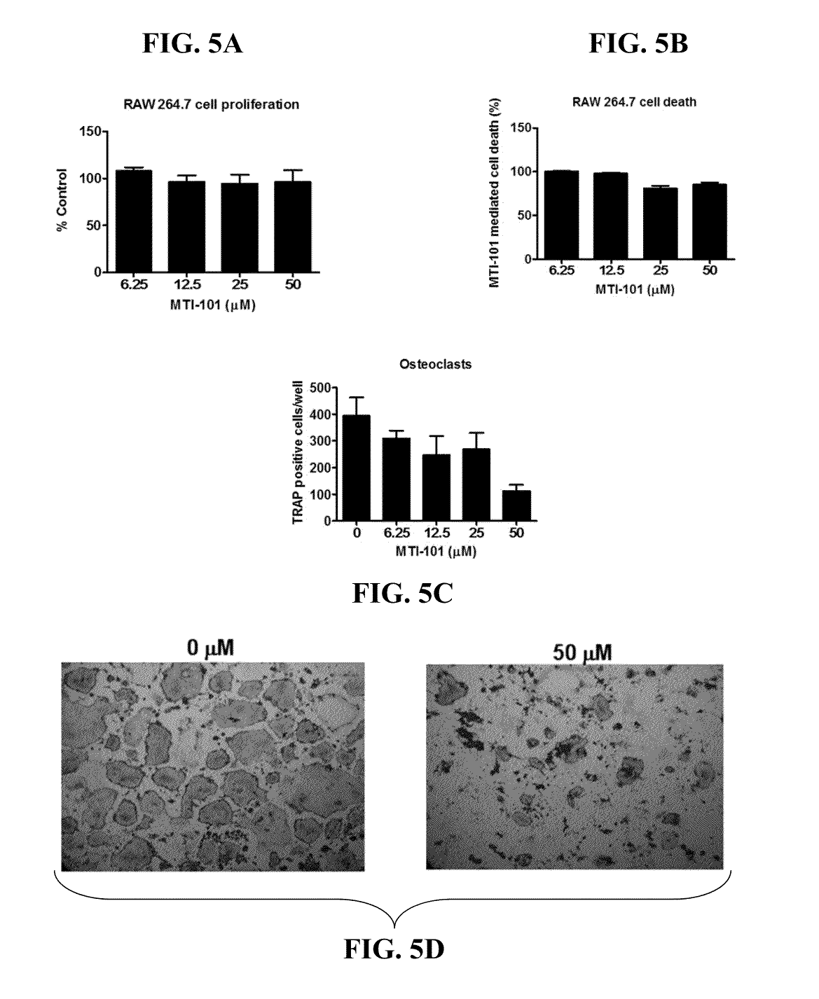

Murine macrophage RAW 264.7 cells were plated at 8×10³ cells/well in a 24 well plate. After overnight incubation, the media was changed (day 1) and cells were stimulated to differentiate into osteoclast by addition of 50 ng/ml of recombinant murine RANKL in the presence or absence of MTI-101. The combination treatment was repeated again on day 3 and day 5 post-cell plating. On day seven, to evaluate the number of osteoclasts formed, a tartarate-resistant acid phosphatase (TRAP) staining was performed using leukocyte acid phosphatase kit (Sigma Aldrich). In parallel, to determine the effect of multiple treatment of MTI-101 on RAW 264.7 cell proliferation and cell death, a MTT and Flow cytometry assay was performed respectively. Results are shown in FIGS. 5A-5C.

Increasing doses of MTI-101 neither affected cell proliferation (FIG. 5A) nor did it induce cell death (FIG. 5B) in RAW 264.7 cells. Indeed, even at the highest dose of 50 μM the cell death and cell proliferation rate was comparable to control untreated cells. However, once the RAW 264.7 cells were differentiated into osteoclasts by stimulation with RANKL, MTI-101 brought about a dose-dependent decrease in the number of osteoblasts. As shown in FIG. 5C, the highest dose of 50 μM MTI-101 brought about a four-fold decrease in the number of osteoclasts as compared to control. This demonstrates that MTI-101 is detrimental to osteoclasts and should have a positive anabolic effect on bone formation.

### Example 6

### Effect of MTI-101 on RAW 264.7 Cells and on RAW 264.7 Cell-Differentiated Osteoclasts

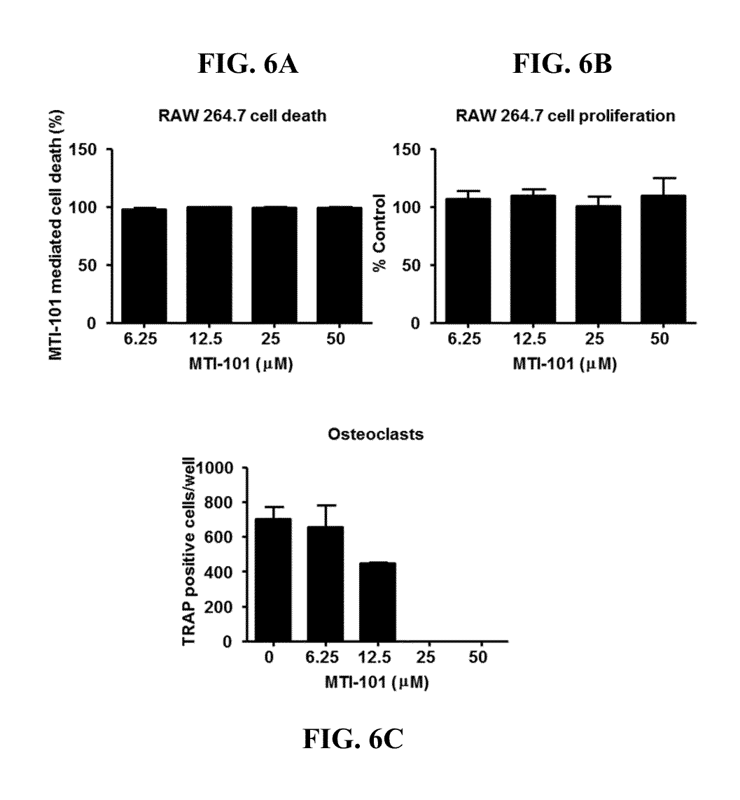

Multiple dosing of MTI-101 neither affected cell proliferation (FIG. 6A) nor did it induce cell death (FIG. 6A) in RAW 264.7 cells. Indeed, even at the highest does of 50 μM the cell death and cell proliferation rate was comparable to control untreated cells. However, MTI-101 did interfere with the process of osteoclastogenesis. MTI-101 dose-dependently inhibited the RANKL-mediated differentiation of RAW 264.7 cells into osteoclasts. As shown in FIG. 6C, this effect of MTI-101 on osteoclastogenesis was very evident at higher doses of 25 and 50 μM, wherein MTI-101 completed inhibited the formation of osteoclasts. These results along with the earlier results demonstrate not only does MTI-101 directly decrease the number of fully differentiated osteoclast, but it also inhibits the process of osteoclastogenesis.

### Example 7

### MTI-101 Reduces Osteoclast Numbers but does not Inhibit Growth or Induce Cell Death in Pre-Osteoclasts

As indicated in Example 5, the inventors utilized RAW 264.7 cells and determined that MTI-101 was devoid of potency in the pre-osteoclast (See FIGS. 5A and 5B) population. In contrast, when RAW 264.7 cells were differentiated to osteoclasts following exposure to RANKL, MTI-101 demonstrated activity. As shown in FIG. 5C, MTI-101 exposure reduced the number of osteoclasts in a dose dependent manner. The mechanism whereby exposure to RANKL sensitizes myeloid cells to MTI-101 induced cell death is to be determined. The CD44 knockout mouse was utilized to determine dependency on CD44 expression on sensitivity to MTI-101 treatment. As shown in FIG. 42, MTI-101 is resistant in reducing osteoclast numbers in CD11b cells derived from the CD44 knockout mouse. The resistance was not complete, indicating that MTI-101 may bind additional link domain family members.

### Example 8

### MTI-101 Reduces Bone Loss in Myeloma Model of Inflammatory Bone Loss

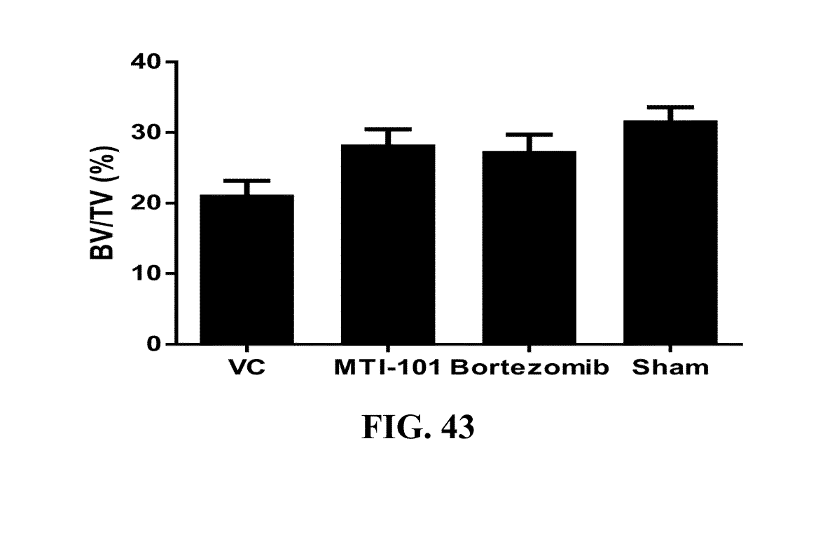

It has previously been determined that MTI-101 increases the survival of mice harboring myeloma tumor burden. More recently, the inventors compared the bone density of MTI-101 and bortezomib treated tumor bearing tibias. Bortezomib, as well as the second generation proteasome inhibitor, carfilzomib, have been reported to induce bone anabolic effects that were demonstrated to be independent of the direct effect on the myeloma cell. Importantly, similar to bortezomib, MTI-101 treatment resulted in increased bone density compared to vehicle control (VC) treated tumor bearing mice. Together, the data indicate that MTI-101 can induce anabolic bone growth in vivo (see FIG. 43).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A method for reducing the number of activated T-cells in a subject, comprising administering an effective amount of a HYD1 peptide to the subject.

2. The method of claim 1, wherein the subject has, or is at risk of developing, an autoimmune disorder.

3. A method for promoting bone preservation in a subject, comprising administering an effective amount of a HYD1 peptide to the subject.

4. The method of claim 3, wherein the subject has, or is at risk of developing, a bone deficiency.

5. A method for treating a bone deficiency and/or an autoimmune disorder in a subject, comprising administering an effective amount of a HYD1 peptide to the subject.

6. The method of claim 1, wherein the HYD1 peptide is a cyclic peptide having a chemical structure shown in FIGS. 7-34 or 38-41, or a pharmaceutically acceptable salt thereof.

7. The method of claim 3, wherein the HYD1 peptide is a cyclic peptide having a chemical structure shown in FIGS. 7-34 or 38-41, or a pharmaceutically acceptable salt thereof.

8. The method of claim 5, wherein the HYD1 peptide is a cyclic peptide having a chemical structure shown in FIGS. 7-34 or 38-41, or a pharmaceutically acceptable salt thereof.

9. The method of claim 6, wherein the cyclic peptide is MTI-101 (shown in FIG. 23), or a pharmaceutically acceptable salt thereof.

10. The method of claim 6, wherein the cyclic peptide has a chemical structure shown in FIGS. 39-41, or a pharmaceutically acceptable salt thereof, wherein R¹ through R⁵ and R⁶ through R¹⁰ are substituents of natural or unnatural amino acids, wherein a sequence of amino acids with R¹ through R⁵ is a non-recognition sequence and a sequence of amino acids with R⁶ through R¹⁰ is a recognition sequence.

11. The method of claim 10, wherein the non-recognition sequence is KLKLK (SEQ ID NO:76), KLQLK (SEQ ID NO:77), QLKLK (SEQ ID NO:78), KQKLK (SEQ ID NO:79), or KXKXK (SEQ ID NO:80) where X=sarcosine where either end of the non-recognition sequence is N-terminus, and wherein the recognition sequence is MVVSW (SEQ ID NO:81), MVVSA (SEQ ID NO:82), MVVAW (SEQ ID NO:83), MVASW (SEQ ID NO:84), MAVSW (SEQ ID NO:85), AVVSW (SEQ ID NO:86), N*VVSW (SEQ ID NO:87), N*VVYW (SEQ ID NO:88), N*VVAW (SEQ ID NO:74), AVVAW (SEQ ID NO:89), N*AVAW (SEQ ID NO:90), N*VAAW (SEQ ID NO:91), N*VLAW (SEQ ID NO:92), N*VIAW (SEQ ID NO:93), N*VFAW (SEQ ID NO:94), or WSVVW (SEQ ID NO:95), where N*=norleucine, where either end of the recognition sequence is N-terminus.

12. The method of claim 10, wherein the non-recognition sequence is KLKLK (SEQ ID NO:76), and wherein the recognition sequence is WAVAW (SEQ ID NO:96), WAVAA (SEQ ID NO:97), WAVAM (SEQ ID NO:98), WAVAN* (SEQ ID NO:99), WAVVN* (SEQ ID NO:75), WAVSN* (SEQ ID NO:100), WAAAW (SEQ ID NO:101), WAAAA (SEQ ID NO:102), WAAAM (SEQ ID NO:103), WAAAN* (SEQ ID NO:104), WAAVW (SEQ ID NO:105), WAAVA (SEQ ID NO:106), WAAVM (SEQ ID NO:107), WAAVN* (SEQ ID NO:108), WAAVSN* (SEQ ID NO:109), WVVAW (SEQ ID NO:110), WVVAA (SEQ ID NO:111), WVVAM (SEQ ID NO:112), WVVAN* (SEQ ID NO:113), WVVVW (SEQ ID NO:114), WVVVA (SEQ ID NO:115), WVVVM (SEQ ID NO:116), WVVVN* (SEQ ID NO:117), WVVSN* (SEQ ID NO:118), WVVAN* (SEQ ID NO:119), WVAVW (SEQ ID NO:120), WVAVA (SEQ ID NO:121), WVAVM (SEQ ID NO:122), WVAVN* (SEQ ID NO:123), WVASN* (SEQ ID NO:124), WSVAW (SEQ ID NO:125), WSVAA (SEQ ID NO:126), WSVAM (SEQ ID NO:127), WSVAN* (SEQ ID NO:128), WSVVW (SEQ ID NO:129), WSVVA (SEQ ID NO:130), WSVVM (SEQ ID NO:131), WSVVN* (SEQ ID NO:132), WSVSW (SEQ ID NO:133), WSVSA (SEQ ID NO:134), WSVSM (SEQ ID NO:135), WSVSN* (SEQ ID NO:136), WSAAW (SEQ ID NO:137), WSAAA (SEQ ID NO:138), WSAAM (SEQ ID NO:139), WSAAN* (SEQ ID NO:140), WSAVW (SEQ ID NO:141), WSAVA (SEQ ID NO:142), WSAVM (SEQ ID NO:143), WSAVN* (SEQ ID NO:144), WSASW (SEQ ID NO:145), WSASA (SEQ ID NO:146), WSASM (SEQ ID NO:147), WSASN* (SEQ ID NO:148), WYVAW (SEQ ID NO:149), WYVAA (SEQ ID NO:150), WYVAM (SEQ ID NO:151), WYVAN* (SEQ ID NO:152), WYVVW (SEQ ID NO:153), WYVVA (SEQ ID NO:154), WYVVM (SEQ ID NO:155), WYVVN* (SEQ ID NO:156), WYVSW (SEQ ID NO:157), WYVSA (SEQ ID NO:158), WYVSM (SEQ ID NO:159), WYVSN* (SEQ ID NO:160), WYAAW (SEQ ID NO:161), WYAAA (SEQ ID NO:162), WYAAM (SEQ ID NO:163), WYAAN* (SEQ ID NO:164), WYAVW (SEQ ID NO:165), WYAVA (SEQ ID NO:166), WYAVM (SEQ ID NO:167), WYAVN* (SEQ ID NO:168), WYASW (SEQ ID NO:169), WYASA (SEQ ID NO:170), WYASM (SEQ ID NO:171), WYASN* (SEQ ID NO:172), AAVAA (SEQ ID NO:173), AAVAM (SEQ ID NO:174), AAVAN* (SEQ ID NO:175), AAVVN* (SEQ ID NO:176), AAVSN* (SEQ ID NO:177), AAAAA (SEQ ID NO:178), AAAAM (SEQ ID NO:179), AAAAN* (SEQ ID NO:180), AAAVW (SEQ ID NO:181), AAAVA (SEQ ID NO:182), AAAVM (SEQ ID NO:183), AAAVN* (SEQ ID NO:184), AAASM (SEQ ID NO:185), AAASN* (SEQ ID NO:186), AVVAW (SEQ ID NO:187), AVVAA (SEQ ID NO:188), AVVAM (SEQ ID NO:189), AVVAN* (SEQ ID NO:190), AVVVA (SEQ ID NO:191), AVVVM (SEQ ID NO:192), AVVVN* (SEQ ID NO:193), AVVSN* (SEQ ID NO:194), AVAAW (SEQ ID NO:195), AVAAM (SEQ ID NO:196), AVAAN* (SEQ ID NO:197), AVAVA (SEQ ID NO:198), AVAVM (SEQ ID NO:199), AVAVN* (SEQ ID NO:200), AVASN* (SEQ ID NO:201), ASVAW (SEQ ID NO:202), ASVAA (SEQ ID NO:203), ASVAM (SEQ ID NO:204), ASVAN* (SEQ ID NO:205), ASVVW (SEQ ID NO:206), ASVVA (SEQ ID NO:207), ASVVM (SEQ ID NO:208), ASVVN* (SEQ ID NO:209), ASVSA (SEQ ID NO:210), ASVSM (SEQ ID NO:211), ASVSN* (SEQ ID NO:212), ASAAW (SEQ ID NO:213), ASAAA (SEQ ID NO:214), ASAAM (SEQ ID NO:215), ASAAN* (SEQ ID NO:216), ASAVW (SEQ ID NO:217), ASAVA (SEQ ID NO:218), ASAVM (SEQ ID NO:219), ASAVN* (SEQ ID NO:220), ASASA (SEQ ID NO:221), ASASM (SEQ ID NO:222), ASASN* (SEQ ID NO:223), AYVAW (SEQ ID NO:224), AYVAA (SEQ ID NO:225), AYVAM (SEQ ID NO:226), AYVAN* (SEQ ID NO:227), AYVVW (SEQ ID NO:228), AYVVA (SEQ ID NO:229), AYVVM (SEQ ID NO:230), AYVVN* (SEQ ID NO:231), AYVSW (SEQ ID NO:232), AYVSA (SEQ ID NO:233), AYVSM (SEQ ID NO:234), AYVSN* (SEQ ID NO:235), AYAAW (SEQ ID NO:236), AYAAA (SEQ ID NO:237), AYAAM (SEQ ID NO:238), AYAAN* (SEQ ID NO:239), AYAVW (SEQ ID NO:240), AYAVA (SEQ ID NO:241), AYAVM (SEQ ID NO:242), AYAVN* (SEQ ID NO:243), AYASW (SEQ ID NO:244), AYASA (SEQ ID NO:245), AYASM (SEQ ID NO:246), AYASN* (SEQ ID NO:247), MAVAA (SEQ ID NO:248), MAVAM (SEQ ID NO:249), MAVAN* (SEQ ID NO:250), MAVVN* (SEQ ID NO:251), MAVSN* (SEQ ID NO:252), MAAAA (SEQ ID NO:253), MAAAM (SEQ ID NO:254), MAAAN* (SEQ ID NO:255), MAAVW (SEQ ID NO:256), MAAVA (SEQ ID NO:257), MAAVM (SEQ ID NO:258), MAAVN* (SEQ ID NO:259), MAASN* (SEQ ID NO:260), MVVAW (SEQ ID NO:261), MVVAA (SEQ ID NO:262), MVVAM (SEQ ID NO:263), MVVAN* (SEQ ID NO:264), MVVVM (SEQ ID NO:265), MVVVN* (SEQ ID NO:266), MVVSN* (SEQ ID NO:267), MVAAM (SEQ ID NO:268), MVAAN* (SEQ ID NO:269), MVAVM (SEQ ID NO:270), MVAVN* (SEQ ID NO:271), MVASN* (SEQ ID NO:272), MSVAW (SEQ ID NO:273), MSVAA (SEQ ID NO:274), MSVAM (SEQ ID NO:275), MSVAN* (SEQ ID NO:276), MSVVW (SEQ ID NO:277), MSVVA (SEQ ID NO:278), MSVVM (SEQ ID NO:279), MSVVN* (SEQ ID NO:280), MSVSM (SEQ ID NO:281), MSVSN* (SEQ ID NO:282), MSAAW (SEQ ID NO:283), MSAAA (SEQ ID NO:284), MSAAM (SEQ ID NO:285), MSAAN* (SEQ ID NO:286), MSAVW (SEQ ID NO:287), MSAVA (SEQ ID NO:288), MSAVM (SEQ ID NO:289), MSAVN* (SEQ ID NO:290), MSASM (SEQ ID NO:291), MSASN* (SEQ ID NO:292), MYVAW (SEQ ID NO:293), MYVAA (SEQ ID NO:294), MYVAM (SEQ ID NO:295), MYVAN* (SEQ ID NO:296) MYVVW (SEQ ID NO:297), MYVVA (SEQ ID NO:298), MYVVM (SEQ ID NO:299), MYVVN* (SEQ ID NO:300), MYVSW (SEQ ID NO:301), MYVSA (SEQ ID NO:302), MYVSM (SEQ ID NO:303), MYVSN* (SEQ ID NO:304), MYAAW (SEQ ID NO:305), MYAAA (SEQ ID NO:306), MYAAM (SEQ ID NO:307), MYAAN* (SEQ ID NO:308), MYAVW (SEQ ID NO:309), MYAVA (SEQ ID NO:310), MYAVM (SEQ ID NO:311), MYAVN* (SEQ ID NO:312), MYASW (SEQ ID NO:313), MYASA (SEQ ID NO:314), MYASM (SEQ ID NO:315), or MYASN* (SEQ ID NO:316), where N*=norleucine, where either end of the recognition sequence is N-terminus.

13. The method of claim 1, wherein the HYD1 peptide is a non-cyclic peptide selected from among: KIKMVISWKG (HYD1; SEQ ID NO:1); AIAMVISWAG (SEQ ID NO:2; HYD8); AIKMVISWAG (SEQ ID NO:3; HYD6); AIKMVISWKG (SEQ ID NO:4; HYD2); AKMVISW (SEQ ID NO:5); AKMVISWKG (SEQ ID NO:6); IAMVISW (SEQ ID NO:7); IAMVISWKG (SEQ ID NO:8); IKAVISW (SEQ ID NO:9); IKAVISWKG (SEQ ID NO:10); IKMAISW (SEQ ID NO:11); IKMAISWKG (SEQ ID NO:12); IKMVASW (SEQ ID NO:13); IKMVASWKG (SEQ ID NO:14); IKMVIAW (SEQ ID NO:15); IKMVIAWKG (SEQ ID NO:16); IKMVISA (SEQ ID NO:17); IKMVISAKG (SEQ ID NO:18); IKMVISW (SEQ ID NO:19); IKMVISWAG (SEQ ID NO:20); KMVISWKA (SEQ ID NO:21); IKMVISWKG (SEQ ID NO:22; HYD18; (-K)HYD1); ISWKG (SEQ ID NO:23); KAKMVISWKG (SEQ ID NO:24); KIAMVISWAG (SEQ ID NO:25; HYD7); KIAMVISWKG (SEQ ID NO:26); KIKAVISWKG (SEQ ID NO:27); KIKMAISWKG (SEQ ID NO:28); KIKMV (SEQ ID NO:29); KIKMVASWKG (SEQ ID NO:30); KIKMVI (SEQ ID NO:31; HYD16); KIKMVIAWKG (SEQ ID NO:32); KIKMVIS (SEQ ID NO:33; HYD15); KIKMVISAKG (SEQ ID NO:34); KIKMVISW (SEQ ID NO:35; HYD14); KIKMVISWAG (SEQ ID NO:36); KIKMVISWK (SEQ ID NO:37; HYD17; HYD1(-G)); KIKMVISWKA (SEQ ID NO:38); KMVISWKG (SEQ ID NO:39; HYD9); LSWKG (SEQ ID NO:40; HYD12); MVISWKG (SEQ ID NO:41; HYD10); SWKG (SEQ ID NO:42; HYD13); VISWKG (SEQ ID NO:43; HYD11); WIKSMKIVKG (SEQ ID NO:44); KMVIXW (SEQ ID NO:45); IKMVISWXX (SEQ ID NO:46); and KMVISWXX (SEQ ID NO:47); wherein X is any amino acid (traditional or non-traditional amino acid).

14. The method of claim 4, wherein the bone deficiency is caused by an osteopenic disorder.

15. The method of claim 14, wherein the osteopenic disorder is selected from among osteoporosis, Paget's disease, lytic bone metastases, periodontitis, rheumatoid arthritis, and bone loss due to immobilization.

16. The method of claim 4, wherein the subject has a cancer that increases osteoclast activity and/or induces bone resorption.

17. The method of claim 1, wherein the subject does not have a proliferation disorder.

18. The method of claim 1, wherein the subject does not have cancer.

19. The method of claim 5, wherein the subject has an autoimmune disorder.

20. The method of claim 2, wherein the autoimmune disorder is selected from among: AIDS-associated myopathy, AIDS-associated neuropathy, Acute disseminated encephalomyelitis, Addison's Disease, Alopecia Areata, Anaphylaxis Reactions, Ankylosing Spondylitis, Antibody-related Neuropathies, Antiphospholipid Syndrome, Arthritis (e.g., rheumatoid arthritis, psoriatic arthritis, juvenile idiopathic arthritis), Autism, Autoimmune Atherosclerosis, Autoimmune Diabetes Insipidus, Autoimmune Endometriosis, Autoimmune Eye Diseases, Autoimmune Gastritis, Autoimmune Hemolytic Anemia, Autoimmune Hemophilia, Auto immune Hepatitis, Auto immune Interstitial Cystitis, Auto immune Lym pho proliferative Syndrome, Autoimmune Myelopathy, Autoimmune Myocarditis, Autoimmune Neuropathies, Autoimmune Oophoritis, Autoimmune Orchitis, Autoimmune Thrombocytopenia, Autoimmune Thyroid Diseases, Autoimmune Urticaria, Autoimmune Uveitis, Autoimmune Vasculitis, Behcet's Disease, Bell's Palsy, Bullous Pemphigoid, CREST, Celiac Disease, Cerebellar degeneration (paraneoplastic), Chronic Fatigue Syndrome, Chronic Rhinosinusitis, Chronic inflammatory demyelinating polyneuropathy, Churg Strauss Syndrome, Connective Tissue Diseases, Crohn's Disease, Cutaneous Lupus, Dermatitis Herpetiformis, Dermatomyositis, Diabetes Mellitus, Discoid Lupus Erythematosus, Drug-induced Lupus, Endocrine Orbitopathy, Glomerulonephritis, Goodpasture Syndrome, Goodpasture's Syndrome, Graft-versus-Host Disease (GVHD), Graves Disease, Guillian-Barre Syndrome, Miller Fisher variant of the Guillian Barre Syndrome, axonal Guillian Barre Syndrome, demyelinating Guillian Barre Syndrome, Hashimoto Thyroiditis, Herpes Gestationis, Human T-cell lymphomavirus-associated myelopathy, Huntington's Disease, IgA Nephropathy, Immune Thrombocytopenic Purpura, Inclusion body myositis, Interstitial Cystitis, Isaacs syndrome, Lambert Eaton myasthenic syndrome, Limbic encephalitis, Lower motor neuron disease, Lyme Disease, MCTD, Microscopic Polyangiitis, Miller Fisher Syndrome, Mixed Connective Tissue Disease, Mononeuritis multiplex (vasculitis), Multiple Sclerosis (relapsing-remitting MS (RRMS), secondary-progressive MS (SPMS), primary-progressive MS (PPMS), and progressive-relapsing MS (PRMS)), Myasthenia Gravis, Myxedema, Meniere Disease, Neonatal LE, Neuropathies with dysproteinemias, Opsoclonus-myoclonus, PBC, POEMS syndrome, Paraneoplastic Autoimmune Syndromes, Pemphigus, Pemphigus Foliaceus, Pemphigus Vulgaris, Pernicious Anemia, Peyronie's Disease, Plaque Psoriasis, Plasmacytoma/myeloma neuropathy, Poly-Dermatomyositis, Polyarteritis Nodosa, Polyendocrine Deficiency Syndrome, Polyendocrine Deficiency Syndrome Type 1, Polyendocrine Deficiency Syndrome Type 2, Polyglandular Autoimmune Syndrome Type I, Polyglandular Autoimmune Syndrome Type II, Polyglandular Autoimmune Syndrome Type III, Polymyositis, Primary Biliary Cirrhosis, Primary Glomerulonephritis, Primary Sclerosing Cholangitis, Psoriasis, Psoriatic Arthritis, Rasmussen's Encephalitis, Raynaud's Disease, Relapsing Polychondritis, Retrobulbar neuritis, Rheumatic Diseases, Rheumatoid Arthritis, Scleroderma, Sensory neuropathies (paraneoplastic), Sjogren's Syndrome, Stiff-Person Syndrome, Subacute Thyroiditis, Subacute autonomic neuropathy, Sydenham Chorea, Sympathetic Ophthalmitis, Systemic Lupus Erythematosus, Transverse myelitis, Type 1 Diabetes, Ulcerative Colitis, Vasculitis, Vitiligo, Wegener's Granulomatosis, acrocyanosis, anaphylacetic reaction, autoimmune inner ear disease, bilateral sensorineural hearing loss, cold agglutinin hemolytic anemia, cold-induced immune hemolytic anemia, idiopathic endolymphatic hydrops, idiopathic progressive bilateral sensorineural hearing loss, immune-mediated inner ear disease, and mixed autoimmune hemolysis.

21. The method of claim 2, wherein the subject has graft-versus-host disease (GVHD), and the HYD1 peptide is administered to treat the GVHD in the subject.

22. The method of claim 2, wherein the HYD1 peptide is administered before, during, and/or after a transplant to delay the onset of graft-versus-host disease (GVHD).

23. The method of claim 22, wherein the transplant comprises an allograft or xenograft.

24. The method of claim 22, wherein the transplant comprises an allogeneic stem cell, bone marrow, or organ transplant.

25. The method of claim 1, further comprising administering a biologically active agent to the subject.

26. The method of claim 25, wherein the agent is selected from among:
(a) an agent for treatment of a bone deficiency, selected from among bisphosphonate, teriparatide, denosumab, and calcitonin; or
(b) an agent for treatment of an autoimmune disorder, selected from among a corticosteroid, nonsteroid drug such as azathioprine, cyclophosphamide, methotrexate, mycophenolate, mofetil, sirolimus, rituximab, tacrolimus, cyclosporine, or other immunosuppressive agent.

27. A composition comprising a HYD1 peptide and one or more agents selected from an agent for treatment of a bone deficiency and/or an autoimmune disorder.

28. The composition of claim 25, wherein the HYD1 peptide is a cyclic peptide having a chemical structure shown in FIGS. 7-34 or 38-41, or a pharmaceutically acceptable salt thereof.

29. The composition of claim 27, wherein the cyclic peptide is MTI-101 (shown in FIGS. 7 and 23), or a pharmaceutically acceptable salt thereof.

30. The composition of claim 28, wherein the cyclic peptide has a chemical structure shown in FIGS. 39-41, or a pharmaceutically acceptable salt thereof, wherein R¹ through R⁵ and R⁶ through R¹⁰ are substituents of natural or unnatural amino acids, wherein a sequence of amino acids with R¹ through R⁵ is a non-recognition sequence and a sequence of amino acids with R⁶ through R¹⁰ is a recognition sequence.

31. The composition of claim 30, wherein the non-recognition sequence is KLKLK (SEQ ID NO:76), KLQLK (SEQ ID NO:77), QLKLK (SEQ ID NO:78), KQKLK (SEQ ID NO:79), or KXKXK (SEQ ID NO:80) where X=sarcosine where either end of the non-recognition sequence is N-terminus, and wherein the recognition sequence is MVVSW (SEQ ID NO:81), MVVSA (SEQ ID NO:82), MVVAW (SEQ ID NO:83), MVASW (SEQ ID NO:84), MAVSW (SEQ ID NO:85), AVVSW (SEQ ID NO:86), N*VVSW (SEQ ID NO:87), N*VVYW (SEQ ID NO:88), N*VVAW (SEQ ID NO:74), AVVAW (SEQ ID NO:89), N*AVAW (SEQ ID NO:90), N*VAAW (SEQ ID NO:91), N*VLAW (SEQ ID NO:92), N*VIAW (SEQ ID NO:93), N*VFAW (SEQ ID NO:94), or WSVVW (SEQ ID NO:95), where N*=norleucine, where either end of the recognition sequence is N-terminus.

32. The composition of claim 30, wherein the non-recognition sequence is KLKLK (SEQ ID NO:76), and wherein the recognition sequence is WAVAW (SEQ ID NO:96), WAVAA (SEQ ID NO:97), WAVAM (SEQ ID NO:98), WAVAN* (SEQ ID NO:99), WAVVN* (SEQ ID NO:75), WAVSN* (SEQ ID NO:100), WAAAW (SEQ ID NO:101), WAAAA (SEQ ID NO:102), WAAAM (SEQ ID NO:103), WAAAN* (SEQ ID NO:104), WAAVW (SEQ ID NO:105), WAAVA (SEQ ID NO:106), WAAVM (SEQ ID NO:107), WAAVN* (SEQ ID NO:108), WAASN* (SEQ ID NO:109), WVVAW (SEQ ID NO:110), WVVAA (SEQ ID NO:111), WVVAM (SEQ ID NO:112), WVVAN* (SEQ ID NO:113), WVVVW (SEQ ID NO:114), WVVVA (SEQ ID NO:115), WVVVM (SEQ ID NO:116), WVVVN* (SEQ ID NO:117), WVVSN* (SEQ ID NO:118), WVAAN* (SEQ ID NO:119), WVAVW (SEQ ID NO:120), WVAVA (SEQ ID NO:121), WVAVM (SEQ ID NO:122), WVAVN* (SEQ ID NO:123), WVASN* (SEQ ID NO:124), WSVAW (SEQ ID NO:125), WSVAA (SEQ ID NO:126), WSVAM (SEQ ID NO:127), WSVAN* (SEQ ID NO:128), WSVVW (SEQ ID NO:129), WSVVA (SEQ ID NO:130), WSVVM (SEQ ID NO:131), WSVVN* (SEQ ID NO:132), WSVSW (SEQ ID NO:133), WSVSA (SEQ ID NO:134), WSVSM (SEQ ID NO:135), WSVSN* (SEQ ID NO:136), WSAAW (SEQ ID NO:137), WSAAA (SEQ ID NO:138), WSAAM (SEQ ID NO:139), WSAAN* (SEQ ID NO:140), WSAVW (SEQ ID NO:141), WSAVA (SEQ ID NO:142), WSAVM (SEQ ID NO:143), WSAVN* (SEQ ID NO:144), WSASW (SEQ ID NO:145), WSASA (SEQ ID NO:146), WSASM (SEQ ID NO:147), WSASN* (SEQ ID NO:148), WYVAW (SEQ ID NO:149), WYVAA (SEQ ID NO:150), WYVAM (SEQ ID NO:151), WYVAN* (SEQ ID NO:152), WYVVW (SEQ ID NO:153), WYVVA (SEQ ID NO:154), WYVVM (SEQ ID NO:155), WYVVN* (SEQ ID NO:156), WYVSW (SEQ ID NO:157), WYVSA (SEQ ID NO:158), WYVSM (SEQ ID NO:159), WYVSN* (SEQ ID NO:160), WYAAW (SEQ ID NO:161), WYAAA (SEQ ID NO:162), WYAAM (SEQ ID NO:163), WYAAN* (SEQ ID NO:164), WYAVW (SEQ ID NO:165), WYAVA (SEQ ID NO:166), WYAVM (SEQ ID NO:167), WYAVN* (SEQ ID NO:168), WYASW (SEQ ID NO:169), WYASA (SEQ ID NO:170), WYASM (SEQ ID NO:171), WYASN* (SEQ ID NO:172), AAVAA (SEQ ID NO:173), AAVAM (SEQ ID NO:174), AAVAN* (SEQ ID NO:175), AAVVN* (SEQ ID NO:176), AAVSN* (SEQ ID NO:177), AAAAA (SEQ ID NO:178), AAAAM (SEQ ID NO:179), AAAAN* (SEQ ID NO:180), AAAVW (SEQ ID NO:181), AAAVA (SEQ ID NO:182), AAAVM (SEQ ID NO:183), AAAVN* (SEQ ID NO:184), AAASM (SEQ ID NO:185), AAASN* (SEQ ID NO:186), AVVAW (SEQ ID NO:187), AVVAA (SEQ ID NO:188), AVVAM (SEQ ID NO:189), AVVAN* (SEQ ID NO:190), AVVVA (SEQ ID NO:191), AVVVM (SEQ ID NO:192), AVVVN* (SEQ ID NO:193), AVVSN* (SEQ ID NO:194), AVAAW (SEQ ID NO:195), AVAAM (SEQ ID NO:196), AVAAN* (SEQ ID NO:197), AVAVA (SEQ ID NO:198), AVAVM (SEQ ID NO:199), AVAVN* (SEQ ID NO:200), AVASN* (SEQ ID NO:201), ASVAW (SEQ ID NO:202), ASVAA (SEQ ID NO:203), ASVAM (SEQ ID NO:204), ASVAN* (SEQ ID NO:205), ASVVW (SEQ ID NO:206), ASVVA (SEQ ID NO:207), ASVVM (SEQ ID NO:208), ASVVN* (SEQ ID NO:209), ASVSA (SEQ ID NO:210), ASVSM (SEQ ID NO:211), ASVSN* (SEQ ID NO:212), ASAAW (SEQ ID NO:213), ASAAA (SEQ ID NO:214), ASAAM (SEQ ID NO:215), ASAAN* (SEQ ID NO:216), ASAVW (SEQ ID NO:217), ASAVA (SEQ ID NO:218), ASAVM (SEQ ID NO:219), ASAVN* (SEQ ID NO:220), ASASA (SEQ ID NO:221), ASASM (SEQ ID NO:222), ASASN* (SEQ ID NO:223), AYVAW (SEQ ID NO:224), AYVAA (SEQ ID NO:225), AYVAM (SEQ ID NO:226), AYVAN* (SEQ ID NO:227), AYVVW (SEQ ID NO:228), AYVVA (SEQ ID NO:229), AYVVM (SEQ ID NO:230), AYVVN* (SEQ ID NO:231), AYVSW (SEQ ID NO:232), AYVSA (SEQ ID NO:233), AYVSM (SEQ ID NO:234), AYVSN* (SEQ ID NO:235), AYAAW (SEQ ID NO:236), AYAAA (SEQ ID NO:237), AYAAM (SEQ ID NO:238), AYAAN* (SEQ ID NO:239), AYAVW (SEQ ID NO:240), AYAVA (SEQ ID NO:241), AYAVM (SEQ ID NO:242), AYAVN* (SEQ ID NO:243), AYASW (SEQ ID NO:244), AYASA (SEQ ID NO:245), AYASM (SEQ ID NO:246), AYASN* (SEQ ID NO:247), MAVAA (SEQ ID NO:248), MAVAM (SEQ ID NO:249), MAVAN* (SEQ ID NO:250), MAVVN* (SEQ ID NO:251), MAVSN* (SEQ ID NO:252), MAAAA (SEQ ID NO:253), MAAAM (SEQ ID NO:254), MAAAN* (SEQ ID NO:255), MAAVW (SEQ ID NO:256), MAAVA (SEQ ID NO:257), MAAVM (SEQ ID NO:258), MAAVN* (SEQ ID NO:259), MAASN* (SEQ ID NO:260), MVVAW (SEQ ID NO:261), MVVAA (SEQ ID NO:262), MVVAM (SEQ ID NO:263), MVVAN* (SEQ ID NO:264), MVVVM (SEQ ID NO:265), MVVVN* (SEQ ID NO:266), MVVSN* (SEQ ID NO:267), MVAAM (SEQ ID NO:268), MVAAN* (SEQ ID NO:269), MVAVM (SEQ ID NO:270), MVAVN* (SEQ ID NO:271), MVASN* (SEQ ID NO:272), MSVAW (SEQ ID NO:273), MSVAA (SEQ ID NO:274), MSVAM (SEQ ID NO:275), MSVAN* (SEQ ID NO:276), MSVVW (SEQ ID NO:277), MSVVA (SEQ ID NO:278), MSVVM (SEQ ID NO:279), MSVVN* (SEQ ID NO:280), MSVSM (SEQ ID NO:281), MSVSN* (SEQ ID NO:282), MSAAW (SEQ ID NO:2831 MSAAA (SEQ ID NO:284), MSAAM (SEQ ID NO:285), MSAAN* (SEQ ID NO:286), MSAVW (SEQ ID NO:287), MSAVA (SEQ ID NO:288), MSAVM (SEQ ID NO:289), MSAVN* (SEQ ID NO:290), MSASM (SEQ TD NO:291), MSASN* (SEQ ID NO:292), MYVAW (SEQ ID NO:293), MYVAA (SEQ ID NO:294), MYVAM (SEQ ID NO:295), MYVAN* (SEQ ID NO:296), MYVVW (SEQ ID NO:297), MYVVA (SEQ ID NO:298), MYVVM (SEQ ID NO:299), MYVVN* (SEQ ID NO:300), MYVSW (SEQ ID NO:301), MYVSA (SEQ ID NO:302), MYVSM (SEQ ID NO:303), MYVSN* (SEQ ID NO:304), MYAAW (SEQ ID NO:305), MYAAA (SEQ ID NO:306), MYAAM (SEQ ID NO:307), MYAAN* (SEQ ID NO:308), MYAVW (SEQ ID NO:309), MYAVA (SEQ ID NO:310), MYAVM (SEQ ID NO:311), MYAVN* (SEQ ID NO:312), MYASW (SEQ ID NO:313), MYASA (SEQ ID NO:314), MYASM (SEQ ID NO:315), or MYASN* (SEQ ID NO:316), where N*=norleucine, where either end of the recognition sequence is N-terminus.

33. The composition of claim 27, wherein the HYD1 peptide is non-cyclic peptide selected from among: KIKMVISWKG (HYD1; SEQ ID NO:1); AIAMVISWAG (SEQ ID NO:2; HYD8); AIKMVISWAG (SEQ ID NO:3; HYDE); AIKMVISWKG (SEQ ID NO:4; HYD2); AKMVISW (SEQ ID NO:5); AKMVISWKG (SEQ ID NO:6); IAMVISW (SEQ ID NO:7); IAMVISWKG (SEQ ID NO:8); IKAVISW (SEQ ID NO:9); IKAVISWKG (SEQ ID NO:10); IKMAISW (SEQ ID NO:11); IKMAISWKG (SEQ ID NO:12); IKMVASW (SEQ ID NO:13); IKMVASWKG (SEQ ID NO:14); IKMVIAW (SEQ ID NO:15); IKMVIAWKG (SEQ ID NO:16); IKMVISA (SEQ ID NO:17); IKMVISAKG (SEQ ID NO:18); IKMVISW (SEQ ID NO:19); IKMVISWAG (SEQ ID NO:20); KMVISWKA (SEQ ID NO:21); IKMVISWKG (SEQ ID NO:22; HYD18; (-K)HYD1); ISWKG (SEQ ID NO:23); KAKMVISWKG (SEQ ID NO:24); KIAMVISWAG (SEQ ID NO:25; HYD7); KIAMVISWKG (SEQ ID NO:26); KIKAVISWKG (SEQ ID NO:27); KIKMAISWKG (SEQ ID NO:28); KIKMV (SEQ ID NO:29); KIKMVASWKG (SEQ ID NO:30); KIKMVI (SEQ ID NO:31; HYD16); KIKMVIAWKG (SEQ ID NO:32); KIKMVIS (SEQ ID NO:33; HYD15); KIKMVISAKG (SEQ ID NO:34); KIKMVISW (SEQ ID NO:35; HYD14); KIKMVISWAG (SEQ ID NO:36); KIKMVISWK (SEQ ID NO:37; HYD17; HYD1(-G)); KIKMVISWKA (SEQ ID NO:38); KMVISWKG (SEQ ID NO:39; HYD9); LSWKG (SEQ ID NO:40; HYD12); MVISWKG (SEQ ID NO:41; HYD10); SWKG (SEQ ID NO:42; HYD13); VISWKG (SEQ ID NO:43; HYD11); WIKSMKIVKG (SEQ ID NO:44); KMVIXW (SEQ ID NO:45); IKMVISWXX (SEQ ID NO:46); and KMVISWXX (SEQ ID NO:47); wherein X is any amino acid (traditional or non-traditional amino acid).

34. The composition of claim 27, wherein the one or more agents are selected from among:
(a) an agent for treatment of a bone deficiency selected from among bisphosphonate, teriparatide, denosumab, and calcitonin; or
(b) an agent for treatment of an autoimmune disorder selected from among a corticosteroid, nonsteroid drug such as azathioprine, cyclophosphamide, methotrexate, mycophenolate, mofetil, sirolimus, rituximab, tacrolimus, cyclosporine, or other immunosuppressive agent.


SEQUENCE LISTING (316 sequences)

SEQ ID NO: 1 - protein, 10 aa, Artificial sequence
  D-amino acid peptide (HYD1)
KIKMVISWKG

SEQ ID NO: 2 - protein, 10 aa, Artificial sequence
  HYD1 variant
AIAMVISWAG

SEQ ID NO: 3 - protein, 10 aa, Artificial sequence
  HYD1 variant
AIKMVISWAG

SEQ ID NO: 4 - protein, 10 aa, Artificial sequence
  HYD1 variant
AIKMVISWKG

SEQ ID NO: 5 - protein, 7 aa, Artificial sequence
  HYD1 variant
AKMVISW

SEQ ID NO: 6 - protein, 9 aa, Artificial sequence
  HYD1 variant
AKMVISWKG

SEQ ID NO: 7 - protein, 7 aa, Artificial sequence
  HYD1 variant
IAMVISW

SEQ ID NO: 8 - protein, 9 aa, Artificial sequence
  HYD1 variant
IAMVISWKG

SEQ ID NO: 9 - protein, 7 aa, Artificial sequence
  HYD1 variant
IKAVISW

SEQ ID NO: 10 - protein, 9 aa, Artificial sequence
  HYD1 variant
IKAVISWKG

SEQ ID NO: 11 - protein, 7 aa, Artificial sequence
  HYD1 variant
IKMAISW

SEQ ID NO: 12 - protein, 9 aa, Artificial sequence
  HYD1 variant
IKMAISWKG

SEQ ID NO: 13 - protein, 7 aa, Artificial sequence
  HYD1 variant
IKMVASW

SEQ ID NO: 14 - protein, 9 aa, Artificial sequence
  HYD1 variant
IKMVASWKG

SEQ ID NO: 15 - protein, 7 aa, Artificial sequence
  HYD1 variant
IKMVIAW

SEQ ID NO: 16 - protein, 9 aa, Artificial sequence
  HYD1 variant
IKMVIAWKG

SEQ ID NO: 17 - protein, 7 aa, Artificial sequence
  HYD1 variant
IKMVISA

SEQ ID NO: 18 - protein, 9 aa, Artificial sequence
  HYD1 variant
IKMVISAKG

SEQ ID NO: 19 - protein, 7 aa, Artificial sequence
  HYD1 fragment
IKMVISW

SEQ ID NO: 20 - protein, 9 aa, Artificial sequence
  HYD1 variant
IKMVISWAG

SEQ ID NO: 21 - protein, 8 aa, Artificial sequence
  HYD1 variant
KMVISWKA

SEQ ID NO: 22 - protein, 9 aa, Artificial sequence
  HYD1 fragment
IKMVISWKG

SEQ ID NO: 23 - protein, 5 aa, Artificial sequence
  HYD1 fragment
ISWKG

SEQ ID NO: 24 - protein, 10 aa, Artificial sequence
  HYD1 variant
KAKMVISWKG

SEQ ID NO: 25 - protein, 10 aa, Artificial sequence
  HYD1 variant
KIAMVISWAG

SEQ ID NO: 26 - protein, 10 aa, Artificial sequence
  HYD1 variant
KIAMVISWKG

SEQ ID NO: 27 - protein, 10 aa, Artificial sequence
  HYD1 variant
KIKAVISWKG

SEQ ID NO: 28 - protein, 10 aa, Artificial sequence
  HYD1 variant
KIKMAISWKG

SEQ ID NO: 29 - protein, 5 aa, Artificial sequence
  HYD1 fragment
KIKMV

SEQ ID NO: 30 - protein, 10 aa, Artificial sequence
  HYD1 variant
KIKMVASWKG

SEQ ID NO: 31 - protein, 6 aa, Artificial sequence
  HYD1 fragment
KIKMVI

SEQ ID NO: 32 - protein, 10 aa, Artificial sequence
  HYD1 variant
KIKMVIAWKG

SEQ ID NO: 33 - protein, 7 aa, Artificial sequence
  HYD1 fragment
KIKMVIS

SEQ ID NO: 34 - protein, 10 aa, Artificial sequence
  HYD1 variant
KIKMVISAKG

SEQ ID NO: 35 - protein, 8 aa, Artificial sequence
  HYD1 fragment
KIKMVISW

SEQ ID NO: 36 - protein, 10 aa, Artificial sequence
  HYD1 variant
KIKMVISWAG

SEQ ID NO: 37 - protein, 9 aa, Artificial sequence
  HYD1 fragment
KIKMVISWK

SEQ ID NO: 38 - protein, 10 aa, Artificial sequence
  HYD1 variant
KIKMVISWKA

SEQ ID NO: 39 - protein, 8 aa, Artificial sequence
  HYD1 fragment
KMVISWKG

SEQ ID NO: 40 - protein, 5 aa, Artificial sequence
  HYD1 variant
LSWKG

SEQ ID NO: 41 - protein, 7 aa, Artificial sequence
  HYD1 fragment
MVISWKG

SEQ ID NO: 42 - protein, 4 aa, Artificial sequence
  HYD1 fragment
SWKG

SEQ ID NO: 43 - protein, 6 aa, Artificial sequence
  HYD1 fragment
VISWKG

SEQ ID NO: 44 - protein, 10 aa, Artificial sequence
  HYD1S (scrambled peptide)
WIKSMKIVKG

SEQ ID NO: 45 - protein, 6 aa, Artificial sequence
  HYD1 variant
KMVIXW

SEQ ID NO: 46 - protein, 9 aa, Artificial sequence
  HYD1 variant
IKMVISWXX

SEQ ID NO: 47 - protein, 8 aa, Artificial sequence
  HYD1 variant
KMVISWXX

SEQ ID NO: 48 - protein, 10 aa, Artificial sequence
  cyclic peptide amino acid sequence
KLKLKMVVSW

SEQ ID NO: 49 - protein, 10 aa, Artificial sequence
  cyclic peptide amino acid sequence
KLKLKMVVSW

SEQ ID NO: 50 - protein, 10 aa, Artificial sequence
  cyclic peptide amino acid sequence
KLKLKMVVSA

SEQ ID NO: 51 - protein, 10 aa, Artificial sequence
  cyclic peptide amino acid sequence
KLKLKMVVAW

SEQ ID NO: 52 - protein, 10 aa, Artificial sequence
  cyclic peptide amino acid sequence
KLKLKMVASW

SEQ ID NO: 53 - protein, 10 aa, Artificial sequence
  cyclic peptide amino acid sequence
KLKLKMAVSW

SEQ ID NO: 54 - protein, 10 aa, Artificial sequence
  cyclic peptide amino acid sequence
KLKLKAVVSW

SEQ ID NO: 55 - protein, 10 aa, Artificial sequence
  cyclic peptide amino acid sequence
KLKLKXVVSW

SEQ ID NO: 56 - protein, 10 aa, Artificial sequence
  cyclic peptide amino acid sequence
KLKLKXVVYW

SEQ ID NO: 57 - protein, 10 aa, Artificial sequence
  cyclic peptide amino acid sequence
KLKLKWSVVM

SEQ ID NO: 58 - protein, 10 aa, Artificial sequence
  cyclic peptide amino acid sequence
KLKLKWAVVX

SEQ ID NO: 59 - protein, 10 aa, Artificial sequence
  cyclic peptide amino acid sequence
KLKLKWAVVA

SEQ ID NO: 60 - protein, 10 aa, Artificial sequence
  cyclic peptide amino acid sequence
KLKLKWAVAX

SEQ ID NO: 61 - protein, 10 aa, Artificial sequence
  cyclic peptide amino acid sequence
KLKLKWAAVX

SEQ ID NO: 62 - protein, 10 aa, Artificial sequence
  cyclic peptide amino acid sequence
KLKLKAAVVX

SEQ ID NO: 63 - protein, 10 aa, Artificial sequence
  cyclic peptide amino acid sequence
KLKLKXVVAW

SEQ ID NO: 64 - protein, 10 aa, Artificial sequence
  cyclic peptide amino acid sequence
KLQLKXVVAW

SEQ ID NO: 65 - protein, 10 aa, Artificial sequence
  cyclic peptide amino acid sequence
QLKLKXVVAW

SEQ ID NO: 66 - protein, 10 aa, Artificial sequence
  cyclic peptide amino acid sequence
KLKLQXVVAW

SEQ ID NO: 67 - protein, 10 aa, Artificial sequence
  cyclic peptide amino acid sequence
KQKLKXVVAW

SEQ ID NO: 68 - protein, 10 aa, Artificial sequence
  cyclic peptide amino acid sequence
KLKQKXVVAW

SEQ ID NO: 69 - protein, 10 aa, Artificial sequence
  cyclic peptide amino acid sequence
KLKLKWAVVX

SEQ ID NO: 70 - protein, 10 aa, Artificial sequence
  cyclic peptide amino acid sequence
KXKXKWAVVX

SEQ ID NO: 71 - protein, 10 aa, Artificial sequence
  cyclic peptide amino acid sequence
KLKLKXVLAW

SEQ ID NO: 72 - protein, 10 aa, Artificial sequence
  cyclic peptide amino acid sequence
KLKLKXVIAW

SEQ ID NO: 73 - protein, 10 aa, Artificial sequence
  cyclic peptide amino acid sequence
KLKLKXVFAW

SEQ ID NO: 74 - protein, 5 aa, Artificial sequence
  amino acid recognition sequence
XVVAW

SEQ ID NO: 75 - protein, 5 aa, Artificial sequence
  amino acid recognition sequence
WAVVX

SEQ ID NO: 76 - protein, 5 aa, Artificial sequence
  amino acid non-recognition sequence
KLKLK

SEQ ID NO: 77 - protein, 5 aa, Artificial sequence
  amino acid non-recognition sequence
KLQLK

SEQ ID NO: 78 - protein, 5 aa, Artificial sequence
  amino acid non-recognition sequence
QLKLK

SEQ ID NO: 79 - protein, 5 aa, Artificial sequence
  amino acid non-recognition sequence
KQKLK

SEQ ID NO: 80 - protein, 5 aa, Artificial sequence
  amino acid non-recognition sequence
KXKXK

SEQ ID NO: 81 - protein, 5 aa, Artificial sequence
  amino acid recognition sequence
MVVSW

SEQ ID NO: 82 - protein, 5 aa, Artificial sequence
  amino acid recognition sequence
MVVSA

SEQ ID NO: 83 - protein, 5 aa, Artificial sequence
  amino acid recognition sequence
MVVAW

SEQ ID NO: 84 - protein, 5 aa, Artificial sequence
  amino acid recognition sequence
MVASW

SEQ ID NO: 85 - protein, 5 aa, Artificial sequence
  amino acid recognition sequence
MAVSW

SEQ ID NO: 86 - protein, 5 aa, Artificial sequence
  amino acid recognition sequence
AVVSW

SEQ ID NO: 87 - protein, 5 aa, Artificial sequence
  amino acid recognition sequence
XVVSW

SEQ ID NO: 88 - protein, 5 aa, Artificial sequence
  amino acid recognition sequence
XVVYW

SEQ ID NO: 89 - protein, 5 aa, Artificial sequence
  amino acid recognition sequence
AVVAW

SEQ ID NO: 90 - protein, 5 aa, Artificial sequence
  amino acid recognition sequence
XAVAW

SEQ ID NO: 91 - protein, 5 aa, Artificial sequence
  amino acid recognition sequence
XVAAW

SEQ ID NO: 92 - protein, 5 aa, Artificial sequence
  amino acid recognition sequence
XVLAW

SEQ ID NO: 93 - protein, 5 aa, Artificial sequence
  amino acid recognition sequence
XVIAW

SEQ ID NO: 94 - protein, 5 aa, Artificial sequence
  amino acid recognition sequence
XVFAW

SEQ ID NO: 95 - protein, 5 aa, Artificial sequence
  amino acid recognition sequence
WSVVW

SEQ ID NO: 96 - protein, 5 aa, Artificial sequence
  amino acid recognition sequence
WAVAW

SEQ ID NO: 97 - protein, 5 aa, Artificial sequence
  amino acid recognition sequence
WAVAA

SEQ ID NO: 98 - protein, 5 aa, Artificial sequence
  amino acid recognition sequence
WAVAM

SEQ ID NO: 99 - protein, 5 aa, Artificial sequence
  amino acid recognition sequence
WAVAX

SEQ ID NO: 100 - protein, 5 aa, Artificial sequence
  amino acid recognition sequence
WAVSX

SEQ ID NO: 101 - protein, 5 aa, Artificial sequence
  amino acid recognition sequence
WAAAW

SEQ ID NO: 102 - protein, 5 aa, Artificial sequence
  amino acid recognition sequence
WAAAA

SEQ ID NO: 103 - protein, 5 aa, Artificial sequence
  amino acid recognition sequence
WAAAM

SEQ ID NO: 104 - protein, 5 aa, Artificial sequence
  amino acid recognition sequence
WAAAX

SEQ ID NO: 105 - protein, 5 aa, Artificial sequence
  amino acid recognition sequence
WAAVW

SEQ ID NO: 106 - protein, 5 aa, Artificial sequence
  amino acid recognition sequence
WAAVA

SEQ ID NO: 107 - protein, 5 aa, Artificial sequence
  amino acid recognition sequence
WAAVM

SEQ ID NO: 108 - protein, 5 aa, Artificial sequence
  amino acid recognition sequence
WAAVX

SEQ ID NO: 109 - protein, 5 aa, Artificial sequence
  amino acid recognition sequence
WAASX

SEQ ID NO: 110 - protein, 5 aa, Artificial sequence
  amino acid recognition sequence
WVVAW

SEQ ID NO: 111 - protein, 5 aa, Artificial sequence
  amino acid recognition sequence
WVVAA

SEQ ID NO: 112 - protein, 5 aa, Artificial sequence
  amino acid recognition sequence
WVVAM

SEQ ID NO: 113 - protein, 5 aa, Artificial sequence
  amino acid recognition sequence
WVVAX

SEQ ID NO: 114 - protein, 5 aa, Artificial sequence
  amino acid recognition sequence
WVVVW

SEQ ID NO: 115 - protein, 5 aa, Artificial sequence
  amino acid recognition sequence
WVVVA

SEQ ID NO: 116 - protein, 5 aa, Artificial sequence
  amino acid recognition sequence
WVVVM

SEQ ID NO: 117 - protein, 5 aa, Artificial sequence
  amino acid recognition sequence
WVVVX

SEQ ID NO: 118 - protein, 5 aa, Artificial sequence
  amino acid recognition sequence
WVVSX

SEQ ID NO: 119 - protein, 5 aa, Artificial sequence
  amino acid recognition sequence
WVAAX

SEQ ID NO: 120 - protein, 5 aa, Artificial sequence
  amino acid recognition sequence
WVAVW

SEQ ID NO: 121 - protein, 5 aa, Artificial sequence
  amino acid recognition sequence
WVAVA

SEQ ID NO: 122 - protein, 5 aa, Artificial sequence
  amino acid recognition sequence
WVAVM

SEQ ID NO: 123 - protein, 5 aa, Artificial sequence
  amino acid recognition sequence
WVAVX

SEQ ID NO: 124 - protein, 5 aa, Artificial sequence
  amino acid recognition sequence
WVASX

SEQ ID NO: 125 - protein, 5 aa, Artificial sequence
  amino acid recognition sequence
WSVAW

SEQ ID NO: 126 - protein, 5 aa, Artificial sequence
  amino acid recognition sequence
WSVAA

SEQ ID NO: 127 - protein, 5 aa, Artificial sequence
  amino acid recognition sequence
WSVAM

SEQ ID NO: 128 - protein, 5 aa, Artificial sequence
  amino acid recognition sequence
WSVAX

SEQ ID NO: 129 - protein, 5 aa, Artificial sequence
  amino acid recognition sequence
WSVVW

SEQ ID NO: 130 - protein, 5 aa, Artificial sequence
  amino acid recognition sequence
WSVVA

SEQ ID NO: 131 - protein, 5 aa, Artificial sequence
  amino acid recognition sequence
WSVVM

SEQ ID NO: 132 - protein, 5 aa, Artificial sequence
  amino acid recognition sequence
WSVVX

SEQ ID NO: 133 - protein, 5 aa, Artificial sequence
  amino acid recognition sequence
WSVSW

SEQ ID NO: 134 - protein, 5 aa, Artificial sequence
  amino acid recognition sequence
WSVSA

SEQ ID NO: 135 - protein, 5 aa, Artificial sequence
  amino acid recognition sequence
WSVSM

SEQ ID NO: 136 - protein, 5 aa, Artificial sequence
  amino acid recognition sequence
WSVSX

SEQ ID NO: 137 - protein, 5 aa, Artificial sequence
  amino acid recognition sequence
WSAAW

SEQ ID NO: 138 - protein, 5 aa, Artificial sequence
  amino acid recognition sequence
WSAAA

SEQ ID NO: 139 - protein, 5 aa, Artificial sequence
  amino acid recognition sequence
WSAAM

SEQ ID NO: 140 - protein, 5 aa, Artificial sequence
  amino acid recognition sequence
WSAAX

SEQ ID NO: 141 - protein, 5 aa, Artificial sequence
  amino acid recognition sequence
WSAVW

SEQ ID NO: 142 - protein, 5 aa, Artificial sequence
  amino acid recognition sequence
WSAVA

SEQ ID NO: 143 - protein, 5 aa, Artificial sequence
  amino acid recognition sequence
WSAVM

SEQ ID NO: 144 - protein, 5 aa, Artificial sequence
  amino acid recognition sequence
WSAVX

SEQ ID NO: 145 - protein, 5 aa, Artificial sequence
  amino acid recognition sequence
WSASW

SEQ ID NO: 146 - protein, 5 aa, Artificial sequence
  amino acid recognition sequence
WSASA

SEQ ID NO: 147 - protein, 5 aa, Artificial sequence
  amino acid recognition sequence
WSASM

SEQ ID NO: 148 - protein, 5 aa, Artificial sequence
  amino acid recognition sequence
WSASX

SEQ ID NO: 149 - protein, 5 aa, Artificial sequence
  amino acid recognition sequence
WYVAW

SEQ ID NO: 150 - protein, 5 aa, Artificial sequence
  amino acid recognition sequence
WYVAA

SEQ ID NO: 151 - protein, 5 aa, Artificial sequence
  amino acid recognition sequence
WYVAM

SEQ ID NO: 152 - protein, 5 aa, Artificial sequence
  amino acid recognition sequence
WYVAX

SEQ ID NO: 153 - protein, 5 aa, Artificial sequence
  amino acid recognition sequence
WYVVW

SEQ ID NO: 154 - protein, 5 aa, Artificial sequence
  amino acid recognition sequence
WYVVA

SEQ ID NO: 155 - protein, 5 aa, Artificial sequence
  amino acid recognition sequence
WYVVM

SEQ ID NO: 156 - protein, 5 aa, Artificial sequence
  amino acid recognition sequence
WYVVX

SEQ ID NO: 157 - protein, 5 aa, Artificial sequence
  amino acid recognition sequence
WYVSW

SEQ ID NO: 158 - protein, 5 aa, Artificial sequence
  amino acid recognition sequence
WYVSA

SEQ ID NO: 159 - protein, 5 aa, Artificial sequence
  amino acid recognition sequence
WYVSM

SEQ ID NO: 160 - protein, 5 aa, Artificial sequence
  amino acid recognition sequence
WYVSX

SEQ ID NO: 161 - protein, 5 aa, Artificial sequence
  amino acid recognition sequence
WYAAW

SEQ ID NO: 162 - protein, 5 aa, Artificial sequence
  amino acid recognition sequence
WYAAA

SEQ ID NO: 163 - protein, 5 aa, Artificial sequence
  amino acid recognition sequence
WYAAM

SEQ ID NO: 164 - protein, 5 aa, Artificial sequence
  amino acid recognition sequence
WYAAX

SEQ ID NO: 165 - protein, 5 aa, Artificial sequence
  amino acid recognition sequence
WYAVW

SEQ ID NO: 166 - protein, 5 aa, Artificial sequence
  amino acid recognition sequence
WYAVA

SEQ ID NO: 167 - protein, 5 aa, Artificial sequence
  amino acid recognition sequence
WYAVM

SEQ ID NO: 168 - protein, 5 aa, Artificial sequence
  amino acid recognition sequence
WYAVX

SEQ ID NO: 169 - protein, 5 aa, Artificial sequence
  amino acid recognition sequence
WYASW

SEQ ID NO: 170 - protein, 5 aa, Artificial sequence
  amino acid recognition sequence
WYASA

SEQ ID NO: 171 - protein, 5 aa, Artificial sequence
  amino acid recognition sequence
WYASM

SEQ ID NO: 172 - protein, 5 aa, Artificial sequence
  amino acid recognition sequence
WYASX

SEQ ID NO: 173 - protein, 5 aa, Artificial sequence
  amino acid recognition sequence
AAVAA

SEQ ID NO: 174 - protein, 5 aa, Artificial sequence
  amino acid recognition sequence
AAVAM

SEQ ID NO: 175 - protein, 5 aa, Artificial sequence
  amino acid recognition sequence
AAVAX

SEQ ID NO: 176 - protein, 5 aa, Artificial sequence
  amino acid recognition sequence
AAVVX

SEQ ID NO: 177 - protein, 5 aa, Artificial sequence
  amino acid recognition sequence
AAVSX

SEQ ID NO: 178 - protein, 5 aa, Artificial sequence
  amino acid recognition sequence
AAAAA

SEQ ID NO: 179 - protein, 5 aa, Artificial sequence
  amino acid recognition sequence
AAAAM

SEQ ID NO: 180 - protein, 5 aa, Artificial sequence
  amino acid recognition sequence
AAAAX

SEQ ID NO: 181 - protein, 5 aa, Artificial sequence
  amino acid recognition sequence
AAAVW

SEQ ID NO: 182 - protein, 5 aa, Artificial sequence
  amino acid recognition sequence
AAAVA

SEQ ID NO: 183 - protein, 5 aa, Artificial sequence
  amino acid recognition sequence
AAAVM

SEQ ID NO: 184 - protein, 5 aa, Artificial sequence
  amino acid recognition sequence
AAAVX

SEQ ID NO: 185 - protein, 5 aa, Artificial sequence
  amino acid recognition sequence
AAASM

SEQ ID NO: 186 - protein, 5 aa, Artificial sequence
  amino acid recognition sequence
AAASX

SEQ ID NO: 187 - protein, 5 aa, Artificial sequence
  amino acid recognition sequence
AVVAW

SEQ ID NO: 188 - protein, 5 aa, Artificial sequence
  amino acid recognition sequence
AVVAA

SEQ ID NO: 189 - protein, 5 aa, Artificial sequence
  amino acid recognition sequence
AVVAM

SEQ ID NO: 190 - protein, 5 aa, Artificial sequence
  amino acid recognition sequence
AVVAX

SEQ ID NO: 191 - protein, 5 aa, Artificial sequence
  amino acid recognition sequence
AVVVA

SEQ ID NO: 192 - protein, 5 aa, Artificial sequence
  amino acid recognition sequence
AVVVM

SEQ ID NO: 193 - protein, 5 aa, Artificial sequence
  amino acid recognition sequence
AVVVX

SEQ ID NO: 194 - protein, 5 aa, Artificial sequence
  amino acid recognition sequence
AVVSX

SEQ ID NO: 195 - protein, 5 aa, Artificial sequence
  amino acid recognition sequence
AVAAW

SEQ ID NO: 196 - protein, 5 aa, Artificial sequence
  amino acid recognition sequence
AVAAM

SEQ ID NO: 197 - protein, 5 aa, Artificial sequence
  amino acid recognition sequence
AVAAX

SEQ ID NO: 198 - protein, 5 aa, Artificial sequence
  amino acid recognition sequence
AVAVA

SEQ ID NO: 199 - protein, 5 aa, Artificial sequence
  amino acid recognition sequence
AVAVM

SEQ ID NO: 200 - protein, 5 aa, Artificial sequence
  amino acid recognition sequence
AVAVX

SEQ ID NO: 201 - protein, 5 aa, Artificial sequence
  amino acid recognition sequence
AVASX

SEQ ID NO: 202 - protein, 5 aa, Artificial sequence
  amino acid recognition sequence
ASVAW

SEQ ID NO: 203 - protein, 5 aa, Artificial sequence
  amino acid recognition sequence
ASVAA

SEQ ID NO: 204 - protein, 5 aa, Artificial sequence
  amino acid recognition sequence
ASVAM

SEQ ID NO: 205 - protein, 5 aa, Artificial sequence
  amino acid recognition sequence
ASVAX

SEQ ID NO: 206 - protein, 5 aa, Artificial sequence
  amino acid recognition sequence
ASVVW

SEQ ID NO: 207 - protein, 5 aa, Artificial sequence
  amino acid recognition sequence
ASVVA

SEQ ID NO: 208 - protein, 5 aa, Artificial sequence
  amino acid recognition sequence
ASVVM

SEQ ID NO: 209 - protein, 5 aa, Artificial sequence
  amino acid recognition sequence
ASVVX

SEQ ID NO: 210 - protein, 5 aa, Artificial sequence
  amino acid recognition sequence
ASVSA

SEQ ID NO: 211 - protein, 5 aa, Artificial sequence
  amino acid recognition sequence
ASVSM

SEQ ID NO: 212 - protein, 5 aa, Artificial sequence
  amino acid recognition sequence
ASVSX

SEQ ID NO: 213 - protein, 5 aa, Artificial sequence
  amino acid recognition sequence
ASAAW

SEQ ID NO: 214 - protein, 5 aa, Artificial sequence
  amino acid recognition sequence
ASAAA

SEQ ID NO: 215 - protein, 5 aa, Artificial sequence
  amino acid recognition sequence
ASAAM

SEQ ID NO: 216 - protein, 5 aa, Artificial sequence
  amino acid recognition sequence
ASAAX

SEQ ID NO: 217 - protein, 5 aa, Artificial sequence
  amino acid recognition sequence
ASAVW

SEQ ID NO: 218 - protein, 5 aa, Artificial sequence
  amino acid recognition sequence
ASAVA

SEQ ID NO: 219 - protein, 5 aa, Artificial sequence
  amino acid recognition sequence
ASAVM

SEQ ID NO: 220 - protein, 5 aa, Artificial sequence
  amino acid recognition sequence
ASAVX

SEQ ID NO: 221 - protein, 5 aa, Artificial sequence
  amino acid recognition sequence
ASASA

SEQ ID NO: 222 - protein, 5 aa, Artificial sequence
  amino acid recognition sequence
ASASM

SEQ ID NO: 223 - protein, 5 aa, Artificial sequence
  amino acid recognition sequence
ASASX

SEQ ID NO: 224 - protein, 5 aa, Artificial sequence
  amino acid recognition sequence
AYVAW

SEQ ID NO: 225 - protein, 5 aa, Artificial sequence
  amino acid recognition sequence
AYVAA

SEQ ID NO: 226 - protein, 5 aa, Artificial sequence
  amino acid recognition sequence
AYVAM

SEQ ID NO: 227 - protein, 5 aa, Artificial sequence
  amino acid recognition sequence
AYVAX

SEQ ID NO: 228 - protein, 5 aa, Artificial sequence
  amino acid recognition sequence
AYVVW

SEQ ID NO: 229 - protein, 5 aa, Artificial sequence
  amino acid recognition sequence
AYVVA

SEQ ID NO: 230 - protein, 5 aa, Artificial sequence
  amino acid recognition sequence
AYVVM

SEQ ID NO: 231 - protein, 5 aa, Artificial sequence
  amino acid recognition sequence
AYVVX

SEQ ID NO: 232 - protein, 5 aa, Artificial sequence
  amino acid recognition sequence
AYVSW

SEQ ID NO: 233 - protein, 5 aa, Artificial sequence
  amino acid recognition sequence
AYVSA

SEQ ID NO: 234 - protein, 5 aa, Artificial sequence
  amino acid recognition sequence
AYVSM

SEQ ID NO: 235 - protein, 5 aa, Artificial sequence
  amino acid recognition sequence
AYVSX

SEQ ID NO: 236 - protein, 5 aa, Artificial sequence
  amino acid recognition sequence
AYAAW

SEQ ID NO: 237 - protein, 5 aa, Artificial sequence
  amino acid recognition sequence
AYAAA

SEQ ID NO: 238 - protein, 5 aa, Artificial sequence
  amino acid recognition sequence
AYAAM

SEQ ID NO: 239 - protein, 5 aa, Artificial sequence
  amino acid recognition sequence
AYAAX

SEQ ID NO: 240 - protein, 5 aa, Artificial sequence
  amino acid recognition sequence
AYAVW

SEQ ID NO: 241 - protein, 5 aa, Artificial sequence
  amino acid recognition sequence
AYAVA

SEQ ID NO: 242 - protein, 5 aa, Artificial sequence
  amino acid recognition sequence
AYAVM

SEQ ID NO: 243 - protein, 5 aa, Artificial sequence
  amino acid recognition sequence
AYAVX

SEQ ID NO: 244 - protein, 5 aa, Artificial sequence
  amino acid recognition sequence
AYASW

SEQ ID NO: 245 - protein, 5 aa, Artificial sequence
  amino acid recognition sequence
AYASA

SEQ ID NO: 246 - protein, 5 aa, Artificial sequence
  amino acid recognition sequence
AYASM

SEQ ID NO: 247 - protein, 5 aa, Artificial sequence
  amino acid recognition sequence
AYASX

SEQ ID NO: 248 - protein, 5 aa, Artificial sequence
  amino acid recognition sequence
MAVAA

SEQ ID NO: 249 - protein, 5 aa, Artificial sequence
  amino acid recognition sequence
MAVAM

SEQ ID NO: 250 - protein, 5 aa, Artificial sequence
  amino acid recognition sequence
MAVAX

SEQ ID NO: 251 - protein, 5 aa, Artificial sequence
  amino acid recognition sequence
MAVVX

SEQ ID NO: 252 - protein, 5 aa, Artificial sequence
  amino acid recognition sequence
MAVSX

SEQ ID NO: 253 - protein, 5 aa, Artificial sequence
  amino acid recognition sequence
MAAAA

SEQ ID NO: 254 - protein, 5 aa, Artificial sequence
  amino acid recognition sequence
MAAAM

SEQ ID NO: 255 - protein, 5 aa, Artificial sequence
  amino acid recognition sequence
MAAAX

SEQ ID NO: 256 - protein, 5 aa, Artificial sequence
  amino acid recognition sequence
MAAVW

SEQ ID NO: 257 - protein, 5 aa, Artificial sequence
  amino acid recognition sequence
MAAVA

SEQ ID NO: 258 - protein, 5 aa, Artificial sequence
  amino acid recognition sequence
MAAVM

SEQ ID NO: 259 - protein, 5 aa, Artificial sequence
  amino acid recognition sequence
MAAVX

SEQ ID NO: 260 - protein, 5 aa, Artificial sequence
  amino acid recognition sequence
MAASX

SEQ ID NO: 261 - protein, 5 aa, Artificial sequence
  amino acid recognition sequence
MVVAW

SEQ ID NO: 262 - protein, 5 aa, Artificial sequence
  amino acid recognition sequence
MVVAA

SEQ ID NO: 263 - protein, 5 aa, Artificial sequence
  amino acid recognition sequence
MVVAM

SEQ ID NO: 264 - protein, 5 aa, Artificial sequence
  amino acid recognition sequence
MVVAX

SEQ ID NO: 265 - protein, 5 aa, Artificial sequence
  amino acid recognition sequence
MVVVM

SEQ ID NO: 266 - protein, 5 aa, Artificial sequence
  amino acid recognition sequence
MVVVX

SEQ ID NO: 267 - protein, 5 aa, Artificial sequence
  amino acid recognition sequence
MVVSX

SEQ ID NO: 268 - protein, 5 aa, Artificial sequence
  amino acid recognition sequence
MVAAM

SEQ ID NO: 269 - protein, 5 aa, Artificial sequence
  amino acid recognition sequence
MVAAX

SEQ ID NO: 270 - protein, 5 aa, Artificial sequence
  amino acid recognition sequence
MVAVM

SEQ ID NO: 271 - protein, 5 aa, Artificial sequence
  amino acid recognition sequence
MVAVX

SEQ ID NO: 272 - protein, 5 aa, Artificial sequence
  amino acid recognition sequence
MVASX

SEQ ID NO: 273 - protein, 5 aa, Artificial sequence
  amino acid recognition sequence
MSVAW

SEQ ID NO: 274 - protein, 5 aa, Artificial sequence
  amino acid recognition sequence
MSVAA

SEQ ID NO: 275 - protein, 5 aa, Artificial sequence
  amino acid recognition sequence
MSVAM

SEQ ID NO: 276 - protein, 5 aa, Artificial sequence
  amino acid recognition sequence
MSVAX

SEQ ID NO: 277 - protein, 5 aa, Artificial sequence
  amino acid recognition sequence
MSVVW

SEQ ID NO: 278 - protein, 5 aa, Artificial sequence
  amino acid recognition sequence
MSVVA

SEQ ID NO: 279 - protein, 5 aa, Artificial sequence
  amino acid recognition sequence
MSVVM

SEQ ID NO: 280 - protein, 5 aa, Artificial sequence
  amino acid recognition sequence
MSVVX

SEQ ID NO: 281 - protein, 5 aa, Artificial sequence
  amino acid recognition sequence
MSVSM

SEQ ID NO: 282 - protein, 5 aa, Artificial sequence
  amino acid recognition sequence
MSVSX

SEQ ID NO: 283 - protein, 5 aa, Artificial sequence
  amino acid recognition sequence
MSAAW

SEQ ID NO: 284 - protein, 5 aa, Artificial sequence
  amino acid recognition sequence
MSAAA

SEQ ID NO: 285 - protein, 5 aa, Artificial sequence
  amino acid recognition sequence
MSAAM

SEQ ID NO: 286 - protein, 5 aa, Artificial sequence
  amino acid recognition sequence
MSAAX

SEQ ID NO: 287 - protein, 5 aa, Artificial sequence
  amino acid recognition sequence
MSAVW

SEQ ID NO: 288 - protein, 5 aa, Artificial sequence
  amino acid recognition sequence
MSAVA

SEQ ID NO: 289 - protein, 5 aa, Artificial sequence
  amino acid recognition sequence
MSAVM

SEQ ID NO: 290 - protein, 5 aa, Artificial sequence
  amino acid recognition sequence
MSAVX

SEQ ID NO: 291 - protein, 5 aa, Artificial sequence
  amino acid recognition sequence
MSASM

SEQ ID NO: 292 - protein, 5 aa, Artificial sequence
  amino acid recognition sequence
MSASX

SEQ ID NO: 293 - protein, 5 aa, Artificial sequence
  amino acid recognition sequence
MYVAW

SEQ ID NO: 294 - protein, 5 aa, Artificial sequence
  amino acid recognition sequence
MYVAA

SEQ ID NO: 295 - protein, 5 aa, Artificial sequence
  amino acid recognition sequence
MYVAM

SEQ ID NO: 296 - protein, 5 aa, Artificial sequence
  amino acid recognition sequence
MYVAX

SEQ ID NO: 297 - protein, 5 aa, Artificial sequence
  amino acid recognition sequence
MYVVW

SEQ ID NO: 298 - protein, 5 aa, Artificial sequence
  amino acid recognition sequence
MYVVA

SEQ ID NO: 299 - protein, 5 aa, Artificial sequence
  amino acid recognition sequence
MYVVM

SEQ ID NO: 300 - protein, 5 aa, Artificial sequence
  amino acid recognition sequence
MYVVX

SEQ ID NO: 301 - protein, 5 aa, Artificial sequence
  amino acid recognition sequence
MYVSW

SEQ ID NO: 302 - protein, 5 aa, Artificial sequence
  amino acid recognition sequence
MYVSA

SEQ ID NO: 303 - protein, 5 aa, Artificial sequence
  amino acid recognition sequence
MYVSM

SEQ ID NO: 304 - protein, 5 aa, Artificial sequence
  amino acid recognition sequence
MYVSX

SEQ ID NO: 305 - protein, 5 aa, Artificial sequence
  amino acid recognition sequence
MYAAW

SEQ ID NO: 306 - protein, 5 aa, Artificial sequence
  amino acid recognition sequence
MYAAA

SEQ ID NO: 307 - protein, 5 aa, Artificial sequence
  amino acid recognition sequence
MYAAM

SEQ ID NO: 308 - protein, 5 aa, Artificial sequence
  amino acid recognition sequence
MYAAX

SEQ ID NO: 309 - protein, 5 aa, Artificial sequence
  amino acid recognition sequence
MYAVW

SEQ ID NO: 310 - protein, 5 aa, Artificial sequence
  amino acid recognition sequence
MYAVA

SEQ ID NO: 311 - protein, 5 aa, Artificial sequence
  amino acid recognition sequence
MYAVM

SEQ ID NO: 312 - protein, 5 aa, Artificial sequence
  amino acid recognition sequence
MYAVX

SEQ ID NO: 313 - protein, 5 aa, Artificial sequence
  amino acid recognition sequence
MYASW

SEQ ID NO: 314 - protein, 5 aa, Artificial sequence
  amino acid recognition sequence
MYASA

SEQ ID NO: 315 - protein, 5 aa, Artificial sequence
  amino acid recognition sequence
MYASM

SEQ ID NO: 316 - protein, 5 aa, Artificial sequence
  amino acid recognition sequence
MYASX